(19) United States
(12) Patent Application Publication
(10) Pub. No.: US 2025/0146015 A1
(43) Pub. Date: May 8, 2025

(54) NON-IMMUNOGENIC CIRCULAR, NON-VIRAL DNA VECTORS

(71) Applicant: Rampart Bioscience, Inc., Monrovia, CA (US)

(72) Inventors: Jeffrey S. Bartlett, Columbus, OH (US); Ming Yan, Encino, CA (US)

(73) Assignee: Rampart Bioscience, Inc., Monrovia, CA (US)

(21) Appl. No.: 19/015,381

(22) Filed: Jan. 9, 2025

Related U.S. Application Data
(63) Continuation of application No. PCT/US2023/070238, filed on Jul. 14, 2023.
(60) Provisional application No. 63/390,515, filed on Jul. 19, 2022.

(51) Int. Cl. C12N 15/85

(52) U.S. Cl. CPC C12N 15/85; C12N 2750/14143; C12N 2800/107; C12N 2820/55

(57) ABSTRACT

The present disclosure relates to circular, non-viral DNA vectors, compositions including one or more of the disclosed vectors, and methods for delivering and/or expressing one or more therapeutic genes (e.g., proteins) in mammals, e.g., human patients. In some embodiments, the present disclosure is directed to circular, non-viral DNA vectors, such as circular non-viral DNA vectors including at least two inverted repeat sequences, where the at least two inverted repeat sequences are separated by a non-repeated nucleotide sequence which is not part of the at least two inverted repeat sequences. In some embodiments, the disclosed circular, non-viral DNA vectors do not include a “DD element.” In some embodiments, the disclosed circular, non-viral DNA vectors do not include a “DD element,” but include at least a portion of a bacterial origin of replication.

### CROSS-REFERENCE TO RELATED APPLICATIONS

This application is a continuation of International Patent Application No. PCT/US2023/070238, filed in the U.S. Receiving Office on Jul. 14, 2023, which claims the benefit of the filing date of U.S. Provisional Patent Application No. 63/390,515 filed on Jul. 19, 2022. The entirety of each of these applications is incorporated herein for all purposes.

### FIELD OF THE DISCLOSURE

The present disclosure relates to non-viral DNA vectors including one or more nucleotide sequences encoding one or more therapeutic proteins. In some embodiments, the non-viral DNA vectors are useful for treating hypophosphatasia or for treating, mitigating, or preventing one or more symptoms of hypophosphatasia in a subject in need of treatment thereof.

### INCORPORATION BY REFERENCE

The contents of the XML file named “24012-004WO1US1_ST26” which was created on Jul. 11, 2023, and is 262 KB in size, are hereby incorporated by reference in their entirety.

### BACKGROUND OF THE DISCLOSURE

Gene therapy is an innovative approach in medicine aimed at treating inherited and acquired diseases through the delivery of new genetic material into a patient's cells to compensate for or suppress the function of a mutant gene and/or treat a genetic disorder.

Vectors used in gene therapy are divided into two main categories, viral and non-viral ones. Viral gene therapy vectors include retroviruses, lentiviruses, adenoviruses, adeno-associated viruses, herpesviruses, poxviruses. (Lundstrom K. Viral Vectors in Gene Therapy. Diseases. 2018 May 21; 6(2)). Viral vectors can actively enter host cells and are transfected efficiently. The main advantage of viral vectors is fast and highly efficient delivery of therapeutic genetic material to a cell due to the natural properties of viruses. Viral vectors, however, have rather limited use in clinical practice given the risk of inflammation, immune response to the gene therapy, cytotoxicity, mutagenesis, and carcinogenesis. There remains a need for non-viral vectors for therapeutic gene delivery.

### BRIEF SUMMARY OF THE DISCLOSURE

The present disclosure relates to circular, non-viral DNA vectors; compositions including one or more of the disclosed circular, non-viral DNA vectors; and methods for delivering and/or expressing one or more therapeutic genes in mammals, e.g., human patients, from the disclosed circular, non-viral DNA. The presently disclosed circular, non-viral DNA vectors are capable of facilitating persistent expression of a transgene which is delivered by the vector to a cell.

A first aspect of the present disclosure is an isolated, circular, non-viral DNA vector comprising: a first portion comprising an expression cassette including one or more nucleic acid sequences encoding one or more therapeutic proteins, where each of the one or more nucleic acid sequences encoding the one or more therapeutic proteins are operatively linked to a promoter; and a second portion capable of forming at least one cruciform structure. In some embodiments, the second portion comprises at least two inverted repeat sequences. In some embodiments, the at least two inverted repeat sequences are separated by a non-repeating nucleotide sequence having at least 3 nucleotides.

In some embodiments, the non-viral DNA vector is substantially devoid of CpG sequences. In some embodiments, the non-viral DNA vector comprises less than about 750 CpG per vector. In some embodiments, the non-viral DNA vector comprises less than about 700 CpG per vector. In some embodiments, the non-viral DNA vector comprises less than about 600 CpG per vector. In some embodiments, the non-viral DNA vector comprises less than about 500 CpG per vector. In some embodiments, the non-viral DNA vector comprises less than about 400 CpG per vector. In some embodiments, the non-viral DNA vector comprises less than about 300 CpG per vector. In some embodiments, the non-viral DNA vector comprises less than about 200 CpG per vector. In some embodiments, the non-viral DNA vector comprises less than about 150 CpG per vector. In some embodiments, the non-viral DNA vector comprises less than about 50 CpG per vector.

In some embodiments, the non-repeating nucleotide sequence comprises at least 5 nucleotides. In some embodiments, the non-repeating nucleotide sequence comprises at least 10 nucleotides. In some embodiments, the non-repeating nucleotide sequence comprises at least 15 nucleotides. In some embodiments, the non-repeating nucleotide sequence comprises at least 20 nucleotides. In some embodiments, the non-repeating nucleotide sequence comprises at least 25 nucleotides. In some embodiments, the non-repeating nucleotide sequence has at least 3 nucleotides and encodes at least a portion of a bacterial origin of replication. In some embodiments, origins of replication include sequences derived from pMB1, pBR322, ColE1, p15A, pSC101, or F1. In some embodiments, the non-repeating nucleotide sequence having the at least 3 nucleotides encodes a heterologous gene or a portion of a heterologous gene. In some embodiments, the non-repeating nucleotide sequence having the at least 3 nucleotides encodes a bacterial suppressor tRNA. In some embodiments, the non-repeating nucleotide sequence having the at least 3 nucleotides encodes a bacterial RNAi repressor. In some embodiments, the non-repeating nucleotide sequence having the at least 3 nucleotides encodes antisense RNA. In some embodiments, the non-repeating nucleotide sequence having the at least 3 nucleotides encodes a bacterial operator sequence or a portion thereof. In some embodiments, the bacterial operator sequence comprises a lac operator. In some embodiments, the bacterial operator sequence comprises a tet operator.

In some embodiments, the non-viral DNA vector lacks a drug resistance gene. In some embodiments, the non-viral DNA vector includes one or more recombination sites. In some embodiments, the one or more recombination sites are selected from the group consisting of LoxP sites, FRT sites, attB and attP sites or their product sites attL or attR, or alternative recombination target sites derived from these sites, e.g., Lox511 or Lox66 sites. In some embodiments, the non-viral DNA vector is substantially double stranded. In some embodiments, the non-viral DNA vector is substantially supercoiled.

In some embodiments, the substantially supercoiled non-viral DNA vectors comprise one or more regions of negative supercoiling. In some embodiments, the non-viral DNA vector is non-immunogenic.

In some embodiments, each of the inverted repeat sequences are derived from nucleic acid sequences present within one or more AAV serotypes. In some embodiments, each of the inverted repeat sequences comprise a nucleotide sequence having at least 85% identity to any one of SEQ ID NOS: 1-18. In some embodiments, each of the inverted repeat sequences comprise a nucleotide sequence having at least 90% identity to any one of SEQ ID NOS: 1-18. In some embodiments, each of the inverted repeat sequences comprises a nucleotide sequence having at least 91% identity to any one of SEQ ID NOS: 1-18. In some embodiments, each of the inverted repeat sequences comprises a nucleotide sequence having at least 92% identity to any one of SEQ ID NOS: 1-18. In some embodiments, each of the inverted repeat sequences comprises a nucleotide sequence having at least 93% identity to any one of SEQ ID NOS: 1-18. In some embodiments, each of the inverted repeat sequences comprises a nucleotide sequence having at least 94% identity to any one of SEQ ID NOS: 1-18. In some embodiments, each of the inverted repeat sequences comprises a nucleotide sequence having at least 95% identity to any one of SEQ ID NOS: 1-18. In some embodiments, each of the inverted repeat sequences comprises a nucleotide sequence having at least 96% identity to any one of SEQ ID NOS: 1-18. In some embodiments, each of the inverted repeat sequences comprises a nucleotide sequence having at least 97% identity to any one of SEQ ID NOS: 1-18. In some embodiments, each of the inverted repeat sequences comprises a nucleotide sequence having at least 98% identity to any one of SEQ ID NOS: 1-18. In some embodiments, each of the inverted repeat sequences comprises a nucleotide sequence having at least 99% identity to any one of SEQ ID NOS: 1-18. In some embodiments, each of the inverted repeat sequences comprises any one of SEQ ID NOS: 1-18.

In some embodiments, the non-viral DNA vectors do not comprise a DD element. In some embodiments, the non-viral DNA vectors do not comprise a DD element but do include at least a portion of a bacterial origin of replication. In some embodiments, the non-viral DNA vectors do not comprise a DD element but do include at least a portion of a bacterial origin of replication as part of the second portion. In some embodiments, the non-viral DNA vectors do not comprise a DD element but do include at least a portion of a bacterial origin of replication, but not within the second portion.

In some embodiments, the non-repeating nucleotide sequence having the least 3 nucleotides has a nucleotide sequence having at least 85% identity to any one of SEQ ID NOS: 58-59. In some embodiments, the non-repeating nucleotide sequence having the least 3 nucleotides has a nucleotide sequence having at least 90% identity to any one of SEQ ID NOS: 58-59. In some embodiments, the non-repeating nucleotide sequence having the least 3 nucleotides has a nucleotide sequence having at least 91% identity to any one of SEQ ID NOS: 58-59. In some embodiments, the non-repeating nucleotide sequence having the least 3 nucleotides has a nucleotide sequence having at least 92% identity to any one of SEQ ID NOS: 58-59. In some embodiments, the non-repeating nucleotide sequence having the least 3 nucleotides has a nucleotide sequence having at least 93% identity to any one of SEQ ID NOS: 58-59. In some embodiments, the non-repeating nucleotide sequence having the least 3 nucleotides has a nucleotide sequence having at least 94% identity to any one of SEQ ID NOS: 58-59. In some embodiments, the non-repeating nucleotide having the least 3 nucleotides has a nucleotide sequence having at least 95% identity to any one of SEQ ID NOS: 58-59. In some embodiments, the non-repeating nucleotide sequence having the least 3 nucleotides has a nucleotide sequence having at least 96% identity to any one of SEQ ID NOS: 58-59. In some embodiments, the non-repeating nucleotide sequence having the least 3 nucleotides has a nucleotide sequence having at least 97% identity to any one of SEQ ID NOS: 58-59. In some embodiments, the non-repeating nucleotide sequence having the least 3 nucleotides has a nucleotide sequence having at least 98% identity to any one of SEQ ID NOS: 58-59. In some embodiments, the non-repeating nucleotide sequence having the least 3 nucleotides has a nucleotide sequence having at least 99% identity to any one of SEQ ID NOS: 58-59. In some embodiments, the non-repeating nucleotide sequence having the least 3 nucleotides has a nucleotide sequence having any one of SEQ ID NOS: 58-59.

In some embodiments, the second portion comprises a first nucleic acid sequence having at least 90% identity to any one of SEQ ID NOS: 1, 3, 5, 7, 9, 11, 13, 15, or 17; a second nucleic acid sequence having at least 90% identity to any one of SEQ ID NOS: 58-59; and a third nucleic acid sequence having at least 90% identity to any one of SEQ ID NOS: 2, 4, 6, 8, 10, 12, 14, 16, or 18. The isolated, circular, non-viral DNA vector of any one of the preceding claims, wherein the second portion comprises a first nucleic acid sequence having at least 92% identity to any one of SEQ ID NOS: 1, 3, 5, 7, 9, 11, 13, 15, or 17; a second nucleic acid sequence having at least 92% identity to any one of SEQ ID NOS: 58-59; and a third nucleic acid sequence having at least 92% identity to any one of SEQ ID NOS: 2, 4, 6, 8, 10, 12, 14, 16, or 18.

The isolated, circular, non-viral DNA vector of any one of the preceding claims, wherein the second portion comprises a first nucleic acid sequence having at least 95% identity to any one of SEQ ID NOS: 1, 3, 5, 7, 9, 11, 13, 15, or 17; a second nucleic acid sequence having at least 95% identity to any one of SEQ ID NOS: 58-59; and a third nucleic acid sequence having at least 95% identity to any one of SEQ ID NOS: 2, 4, 6, 8, 10, 12, 14, 16, or 18. The isolated, circular, non-viral DNA vector of any one of the preceding claims, wherein the second portion comprises a first nucleic acid sequence having at least 96% identity to any one of SEQ ID NOS: 1, 3, 5, 7, 9, 11, 13, 15, or 17; a second nucleic acid sequence having at least 96% identity to any one of SEQ ID NOS: 58-59; and a third nucleic acid sequence having at least 96% identity to any one of SEQ ID NOS: 2, 4, 6, 8, 10, 12, 14, 16, or 18. The isolated, circular, non-viral DNA vector of any one of the preceding claims, wherein the second portion comprises a first nucleic acid sequence having at least 97% identity to any one of SEQ ID NOS: 1, 3, 5, 7, 9, 11, 13, 15, or 17; a second nucleic acid sequence having at least 97% identity to any one of SEQ ID NOS: 58-59; and a third nucleic acid sequence having at least 97% identity to any one of SEQ ID NOS: 2, 4, 6, 8, 10, 12, 14, 16, or 18. The isolated, circular, non-viral DNA vector of any one of the preceding claims, wherein the second portion comprises a first nucleic acid sequence having at least 98% identity to any one of SEQ ID NOS: 1, 3, 5, 7, 9, 11, 13, 15, or 17; a second nucleic acid sequence having at least 98% identity to any one of SEQ ID NOS: 58-59; and a third nucleic acid sequence having at least 98% identity to any one of SEQ ID NOS: 2, 4, 6, 8, 10, 12, 14, 16, or 18. The isolated, circular, non-viral DNA vector of any one of the preceding claims, wherein the second portion comprises a first nucleic acid sequence having at least 99% identity to any one of SEQ ID NOS: 1, 3, 5, 7, 9, 11, 13, 15, or 17; a second nucleic acid sequence having at least 99% identity to any one of SEQ ID NOS: 58-59; and a third nucleic acid sequence having at least 99% identity to any one of SEQ ID NOS: 2, 4, 6, 8, 10, 12, 14, 16, or 18.

In some embodiments, the second portion comprises a first nucleic acid sequence having any one of SEQ ID NOS: 1, 3, 5, 7, 9, 11, 13, 15, or 17; a second nucleic acid sequence having any one of SEQ ID NOS: 58-59 and a third nucleic acid sequence having any one of SEQ ID NOS: 2, 4, 6, 8, 10, 12, 14, 16, or 18.

In some embodiments, the second portion comprises a nucleic acid sequence having at least 90% identity to any one of SEQ ID NOS: 35 and 60. In some embodiments, the second portion comprises a nucleic acid sequence having at least 95% identity to any one of SEQ ID NOS: 35 and 60. In some embodiments, the second portion comprises a nucleic acid sequence having at least 96% identity to any one of SEQ ID NOS: 35 and 60. In some embodiments, the second portion comprises a nucleic acid sequence having at least 97% identity to any one of SEQ ID NOS: 35 and 60. In some embodiments, the second portion comprises a nucleic acid sequence having at least 98% identity to any one of SEQ ID NOS: 35 and 60. In some embodiments, the second portion comprises a nucleic acid sequence having at least 99% identity to any one of SEQ ID NOS: 35 and 60. In some embodiments, the second portion comprises a nucleic acid sequence having any one of SEQ ID NOS: 35 and 60.

In some embodiments, the one or more therapeutic proteins are selected from the group consisting of ALPL, PCSK9, PCSK7, SerpinA1, ABCB4 (having at least 80%, at least 85%, at least 90%, at least 95%, at least 97%, at least 99% identity to any one of SEQ ID NOS: 64, 65, 66, or 69), ATP7B (having at least 80%, at least 85%, at least 90%, at least 95%, at least 97%, at least 99% identity to any one of SEQ ID NOS: 70 or 83), A1AT (having at least 80%, at least 85%, at least 90%, at least 95%, at least 97%, at least 99% identity to SEQ ID NO: 66), ABCB11, antiCD19-antiCD3 (having at least 80%, at least 85%, at least 90%, at least 95%, at least 97%, at least 99% identity to SEQ ID NO: 67), BDF8 (having at least 80%, at least 85%, at least 90%, at least 95%, at least 97%, at least 99% identity to SEQ ID NO: 68) and variants thereof. In some embodiments, the one or more therapeutic proteins are alkaline phosphatase or a variant thereof.

A second aspect of the present disclosure is an isolated, circular, non-viral DNA vector comprising: a first portion comprising an expression cassette including one or more nucleic acid sequences encoding one or more therapeutic proteins, where each of the one or more nucleic acid sequences encoding the one or more therapeutic proteins are operatively linked to a promoter; and a second portion capable of forming at least one cruciform structure, wherein the second portion has the Formula X—Y—X′, where X and X′ are each inverted repeat sequences, and where Y is not repeated and comprises a nucleotide sequence having at least 3 nucleotides.

In some embodiments, the non-viral DNA vector is substantially devoid of CpG sequences. In some embodiments, the non-viral DNA vector comprises less than about 400 CpG per vector. In some embodiments, the non-viral DNA vector comprises less than about 300 CpG per vector. In some embodiments, the non-viral DNA vector comprises less than about 200 CpG per vector. In some embodiments, the non-viral DNA vector comprises less than about 150 CpG per vector. In some embodiments, the non-viral DNA vector comprises less than about 50 CpG per vector.

In some embodiments, Y comprises at least 5 nucleotides. In some embodiments, Y comprises at least 10 nucleotides. In some embodiments, Y comprises at least 15 nucleotides. In some embodiments, Y comprises at least 20 nucleotides. In some embodiments, Y encodes at least a portion of a bacterial origin of replication. In some embodiments, Y encodes a heterologous gene or a portion of a heterologous gene. In some embodiments, Y encodes a bacterial suppressor tRNA. In some embodiments, Y encodes a bacterial RNAi repressor. In some embodiments, Y encodes antisense RNA. In some embodiments, Y encodes a bacterial operator sequence. In some embodiments, the bacterial operator sequence comprises a lac operator. In some embodiments, the bacterial operator sequence comprises a tet operator.

In some embodiments, the non-viral DNA vector lacks a drug resistance gene. In some embodiments, the non-viral DNA vector includes one or more recombination sites. In some embodiments, the one or more recombination sites are selected from the group consisting of LoxP sites, FRT sites, attB and attP sites or their product sites attL or attR, or alternative recombination target sites derived from these sites, e.g., Lox511 or Lox66 sites. In some embodiments, the non-viral DNA vector is substantially double stranded. In some embodiments, the non-viral DNA vector is substantially supercoiled. In some embodiments, the substantially supercoiled non-viral DNA vectors comprise one or more regions of negative supercoiling. In some embodiments, the non-viral DNA vector is non-immunogenic.

In some embodiments, X and X′ are derived from nucleic acid sequences present within one or more AAV serotypes. In some embodiments, X and X′ each comprise a nucleotide sequence having at least 85% identity to any one of SEQ ID NOS: 1-18. In some embodiments, X and X′ each comprise a nucleotide sequence having at least 90% identity to any one of SEQ ID NOS: 1-18. In some embodiments, X and X′ each comprise a nucleotide sequence having at least 91% identity to any one of SEQ ID NOS: 1-18. In some embodiments, X and X′ each comprise a nucleotide sequence having at least 92% identity to any one of SEQ ID NOS: 1-18. In some embodiments, X and X′ each comprise a nucleotide sequence having at least 93% identity to any one of SEQ ID NOS: 1-18. In some embodiments, X and X′ each comprise a nucleotide sequence having at least 94% identity to any one of SEQ ID NOS: 1-18. In some embodiments, X and X′ each comprise a nucleotide sequence having at least 95% identity to any one of SEQ ID NOS: 1-18. In some embodiments, X and X′ each comprise a nucleotide sequence having at least 96% identity to any one of SEQ ID NOS: 1-18. In some embodiments, X and X′ each comprise a nucleotide sequence having at least 97% identity to any one of SEQ ID NOS: 1-18. In some embodiments, X and X′ each comprise a nucleotide sequence having at least 98% identity to any one of SEQ ID NOS: 1-18. In some embodiments, X and X′ each comprise a nucleotide sequence having at least 99% identity to any one of SEQ ID NOS: 1-18. In some embodiments, the non-viral DNA vectors do not comprise a DD element. In some embodiments, the non-viral DNA vectors do not comprise a DD element but do include at least a portion of a bacterial origin of replication.

In some embodiments, Y has a nucleotide sequence having at least 85% identity to any one of SEQ ID NOS: 58-59. In some embodiments, Y has a nucleotide sequence having at least 90% identity to any one of SEQ ID NOS: 58-59. In some embodiments, Y has a nucleotide sequence having at least 91% identity to any one of SEQ ID NOS: 58-59. In some embodiments, Y has a nucleotide sequence having at least 92% identity to any one of SEQ ID NOS: 58-59. In some embodiments, Y has a nucleotide sequence having at least 93% identity to any one of SEQ ID NOS: 58-59. In some embodiments, Y has a nucleotide sequence having at least 94% identity to any one of SEQ ID NOS: 58-59. In some embodiments, Y has a nucleotide sequence having at least 95% identity to any one of SEQ ID NOS: 58-59. In some embodiments, Y has a nucleotide sequence having at least 96% identity to any one of SEQ ID NOS: 58-59. In some embodiments, Y has a nucleotide sequence having at least 97% identity to any one of SEQ ID NOS: 58-59. In some embodiments, Y has a nucleotide sequence having at least 98% identity to any one of SEQ ID NOS: 58-59. In some embodiments, Y has a nucleotide sequence having at least 99% identity to any one of SEQ ID NOS: 58-59. In some embodiments, Y has a nucleotide sequence having any one of SEQ ID NOS: 58-59. In some embodiments, Y does not include a bacterial origin of replication or any portion thereof.

In some embodiments, the non-viral DNA vector includes a bacterial origin or replication or a portion of a bacterial origin of replication, but where the bacterial origin of replication or the portion of the bacterial origin of replication is not included in the second portion. In some embodiments, the non-viral DNA vector includes a bacterial origin or replication or a portion of a bacterial origin of replication, but where the bacterial origin of replication or the portion of the bacterial origin of replication is not included in Y In some embodiments, the one or more therapeutic proteins are selected from the group consisting of ALPL, PCSK9, PCSK7, SerpinAl, ABCB4 (having at least 80%, at least 85%, at least 90%, at least 95%, at least 97%, at least 99% identity to any one of SEQ ID NOS: 64 or 65), ATP7B (having at least 80%, at least 85%, at least 90%, at least 95%, at least 97%, at least 99% identity to any one of SEQ ID NOS: 70 or 83), AlAT (having at least 80%, at least 85%, at least 90%, at least 95%, at least 97%, at least 99% identity to SEQ ID NO: 66), ABCB11, antiCD19-antiCD3 (having at least 80%, at least 85%, at least 90%, at least 95%, at least 97%, at least 99% identity to SEQ ID NO: 67), BDF8 (having at least 80%, at least 85%, at least 90%, at least 95%, at least 97%, at least 99% identity to SEQ ID NO: 68) and variants thereof. In some embodiments, the one or more therapeutic proteins are an alkaline phosphatase or a variant thereof.

A third aspect of the present disclosure is an isolated, circular, non-viral DNA vector comprising the following elements operatively linked in a 5′ to a 3′ direction: (i) a first repeat sequence; (ii) a non-repeating nucleotide sequence having at least 3 nucleotides; (iii) a second repeat sequence; and (iv) an expression cassette. In some embodiments, the non-viral DNA vector comprises less than about 400 CpG per vector. In some embodiments, the non-viral DNA vector comprises less than about 300 CpG per vector. In some embodiments, the non-viral DNA vector comprises less than about 250 CpG per vector. In some embodiments, the non-viral DNA vector comprises less than about 200 CpG per vector. In some embodiments, the non-viral DNA vector comprises less than about 150 CpG per vector. In some embodiments, the non-viral DNA vector comprises less than about 100 CpG per vector. In some embodiments, the non-viral DNA vector comprises less than about 50 CpG per vector. In some embodiments, the expression cassette includes one or more nucleic acid sequences encoding one or more therapeutic proteins, where each of the one or more nucleic acid sequences encoding the one or more therapeutic proteins are operatively linked to a promoter.

In some embodiments, the non-repeating nucleotide sequence comprises at least 5 nucleotides. In some embodiments, the non-repeating nucleotide sequence comprises at least 10 nucleotides. In some embodiments, the non-repeating nucleotide sequence comprises at least 15 nucleotides. In some embodiments, the non-repeating nucleotide sequence comprises at least 20 nucleotides. In some embodiments, the non-repeating nucleotide sequence having the at least 3 nucleotides encodes at least a portion of a bacterial origin of replication. In some embodiments, the non-repeating nucleotide sequence having the at least 3 nucleotides encodes a heterologous gene or a portion of a heterologous gene. In some embodiments, the non-repeating nucleotide sequence having the at least 3 nucleotides encodes a bacterial suppressor tRNA. In some embodiments, the non-repeating nucleotide sequence having the at least 3 nucleotides encodes a bacterial suppressor RNAi repressor. In some embodiments, the non-repeating nucleotide sequence having the at least 3 nucleotides encodes antisense RNA. In some embodiments, the non-repeating nucleotide sequence having the at least 3 nucleotides encodes a bacterial operator sequence. In some embodiments, the bacterial operator sequence comprises a lac operator. In some embodiments, the bacterial operator sequence comprises a tet operator.

In some embodiments, the non-viral DNA vector lacks a drug resistance gene. In some embodiments, the non-viral DNA vector includes one or more recombination sites. In some embodiments, the one or more recombination sites are selected from the group consisting of LoxP sites, FRT sites, attB and attP sites or their product sites attL or attR, or alternative recombination target sites derived from these sites, e.g., Lox511 or Lox66 sites. In some embodiments, the non-viral DNA vector is non-immunogenic. In some embodiments, the non-viral DNA vector is substantially double stranded.

In some embodiments, the one or more therapeutic proteins are selected from the group consisting of ALPL, PCSK9, PCSK7, SerpinAl, ABCB4 (having at least 80%, at least 85%, at least 90%, at least 95%, at least 97%, at least 99% identity to any one of SEQ ID NOS: 64, 65, 66, or 69), ATP7B (having at least 80%, at least 85%, at least 90%, at least 95%, at least 97%, at least 99% identity to any one of SEQ ID NOS: 70 or 83), AlAT (having at least 80%, at least 85%, at least 90%, at least 95%, at least 97%, at least 99% identity to SEQ 1D NO: 66), ABCB11, antiCD19-antiCD3 (having at least 80%, at least 85%, at least 90%, at least 95%, at least 97%, at least 99% identity to SEQ ID NO: 67), BDF8 (having at least 80%, at least 85%, at least 90%, at least 95%, at least 97%, at least 99% identity to SEQ ID NO: 68) and their variants thereof. In some embodiments, the one or more therapeutic proteins are an alkaline phosphatase or a variant thereof.

A fourth aspect of the present disclosure is an isolated, circular, non-viral DNA vector comprising: a first portion comprising an expression cassette including one or more nucleic acid sequences encoding one or more therapeutic proteins, where each of the one or more nucleic acid sequences encoding the one or more therapeutic proteins are operatively linked to a promoter; and a second portion capable of forming at least one cruciform structure, wherein the second portion comprises at least two inverted repeat sequences, and where the at least two inverted repeat sequences are separated by at least a portion of a bacterial origin of replication.

In some embodiments, the non-viral DNA vector is substantially devoid of CpG sequences. In some embodiments, the non-viral DNA vector comprises less than about 400 CpG per vector. In some embodiments, the non-viral DNA vector comprises less than about 300 CpG per vector. In some embodiments, the non-viral DNA vector comprises less than about 250 CpG per vector. In some embodiments, the non-viral DNA vector comprises less than about 200 CpG per vector. In some embodiments, the non-viral DNA vector comprises less than about 150 CpG per vector. In some embodiments, the non-viral DNA vector comprises less than about 50 CpG per vector.

In some embodiments, the non-viral DNA vector lacks a drug resistance gene. In some embodiments, the non-viral DNA vector includes one or more recombination sites. In some embodiments, the one or more recombination sites are selected from the group consisting of LoxP sites, FRT sites, attB and attP sites or their product sites attL or attR, or alternative recombination target sites derived from these sites, e.g., Lox511 or Lox66 sites. In some embodiments, the non-viral DNA vector is substantially double stranded. In some embodiments, the non-viral DNA vector is substantially supercoiled.

In some embodiments, the substantially supercoiled non-viral DNA vectors comprise one or more regions of negative supercoiling. In some embodiments, the non-viral DNA vector is non-immunogenic.

In some embodiments, each of the inverted repeat sequences are derived from nucleic acid sequences present within one or more AAV serotypes. In some embodiments, each of the inverted repeat sequences comprise a nucleotide sequence having at least 85% identity to any one of SEQ ID NOS: 1-18. In some embodiments, each of the inverted repeat sequences comprise a nucleotide sequence having at least 90% identity to any one of SEQ ID NOS: 1-18. In some embodiments, each of the inverted repeat sequences comprise a nucleotide sequence having at least 91% identity to any one of SEQ ID NOS: 1-18. In some embodiments, each of the inverted repeat sequences comprise a nucleotide sequence having at least 92% identity to any one of SEQ ID NOS: 1-18. In some embodiments, each of the inverted repeat sequences comprise a nucleotide sequence having at least 93% identity to any one of SEQ ID NOS: 1-18. In some embodiments, each of the inverted repeat sequences comprise a nucleotide sequence having at least 94% identity to any one of SEQ ID NOS: 1-18. In some embodiments, each of the inverted repeat sequences comprise a nucleotide sequence having at least 95% identity to any one of SEQ ID NOS: 1-18. In some embodiments, each of the inverted repeat sequences comprise a nucleotide sequence having at least 96% identity to any one of SEQ ID NOS: 1-18. In some embodiments, each of the inverted repeat sequences comprise a nucleotide sequence having at least 97% identity to any one of SEQ ID NOS: 1-18. In some embodiments, each of the inverted repeat sequences comprise a nucleotide sequence having at least 98% identity to any one of SEQ ID NOS: 1-18. In some embodiments, each of the inverted repeat sequences comprise a nucleotide sequence having at least 99% identity to any one of SEQ ID NOS: 1-18. In some embodiments, the non-viral DNA vectors do not comprise a DD element. In some embodiments, the non-viral DNA vectors do not comprise a DD element but do include at least a portion of a bacterial origin of replication.

In some embodiments, the at least the portion of a bacterial origin of replication has a nucleotide sequence having at least 85% identity to any one of SEQ ID NOS: 58-59. In some embodiments, at least the portion of a bacterial origin of replication has a nucleotide sequence having at least 90% identity to any one of SEQ ID NOS: 58-59. In some embodiments, the at least the portion of a bacterial origin of replication has a nucleotide sequence having at least 95% identity to any one of SEQ ID NOS: 58-59. In some embodiments, the at least the portion of a bacterial origin of replication has a nucleotide sequence having at least 96% identity to any one of SEQ ID NOS: 58-59. In some embodiments, the at least the portion of a bacterial origin of replication has a nucleotide sequence having at least 97% identity to any one of SEQ ID NOS: 58-59. In some embodiments, the at least the portion of a bacterial origin of replication has a nucleotide sequence having at least 98% identity to any one of SEQ ID NOS: 58-59. In some embodiments, the at least the portion of a bacterial origin of replication has a nucleotide sequence having at least 99% identity to any one of SEQ ID NOS: 58-59. In some embodiments, at least the portion of a bacterial origin of replication has a nucleotide sequence having any one of SEQ ID NOS: 58-59.

In some embodiments, the second portion comprises a first nucleic acid sequence having at least 85% identity to any one of SEQ ID NOS: 1, 3, 5, 7, 9, 11, 13, 15, or 17; a second nucleic acid sequence having at least 85% identity to any one of SEQ ID NOS: 58-59; and a third nucleic acid sequence having at least 85% identity to any one of SEQ ID NOS: 2, 4, 6, 8, 10, 12, 14, 16, or 18. In some embodiments, the second portion comprises a first nucleic acid sequence having at least 90% identity to any one of SEQ ID NOS: 1, 3, 5, 7, 9, 11, 13, 15, or 17; a second nucleic acid sequence having at least 90% identity to any one of SEQ ID NOS: 58-59; and a third nucleic acid sequence having at least 90% identity to any one of SEQ ID NOS: 2, 4, 6, 8, 10, 12, 14, 16, or 18. In some embodiments, the second portion comprises a first nucleic acid sequence having at least 95% identity to any one of SEQ ID NOS: 1, 3, 5, 7, 9, 11, 13, 15, or 17; a second nucleic acid sequence having at least 95% identity to any one of SEQ ID NOS: 58-59; and a third nucleic acid sequence having at least 95% identity to any one of SEQ ID NOS: 2, 4, 6, 8, 10, 12, 14, 16, or 18. In some embodiments, the second portion comprises a first nucleic acid sequence having at least 96% identity to any one of SEQ ID NOS: 1, 3, 5, 7, 9, 11, 13, 15, or 17; a second nucleic acid sequence having at least 96% identity to any one of SEQ ID NOS: 58-59; and a third nucleic acid sequence having at least 96% identity to any one of SEQ ID NOS: 2, 4, 6, 8, 10, 12, 14, 16, or 18. In some embodiments, the second portion comprises a first nucleic acid sequence having at least 97% identity to any one of SEQ ID NOS: 1, 3, 5, 7, 9, 11, 13, 15, or 17; a second nucleic acid sequence having at least 97% identity to any one of SEQ ID NOS: 58-59; and a third nucleic acid sequence having at least 97% identity to any one of SEQ ID NOS: 2, 4, 6, 8, 10, 12, 14, 16, or 18. In some embodiments, the second portion comprises a first nucleic acid sequence having at least 98% identity to any one of SEQ ID NOS: 1, 3, 5, 7, 9, 11, 13, 15, or 17; a second nucleic acid sequence having at least 98% identity to any one of SEQ ID NOS: 58-59; and a third nucleic acid sequence having at least 98% identity to any one of SEQ ID NOS: 2, 4, 6, 8, 10, 12, 14, 16, or 18. In some embodiments, the second portion comprises a first nucleic acid sequence having at least 99% identity to any one of SEQ ID NOS: 1, 3, 5, 7, 9, 11, 13, 15, or 17; a second nucleic acid sequence having at least 99% identity to any one of SEQ ID NOS: 58-59; and a third nucleic acid sequence having at least 99% identity to any one of SEQ ID NOS: 2, 4, 6, 8, 10, 12, 14, 16, or 18. In some embodiments, the second portion comprises a first nucleic acid sequence having any one of SEQ ID NOS: 1, 3, 5, 7, 9, 11, 13, 15, or 17; a second nucleic acid sequence having any one of SEQ ID NOS: 58-59; and a third nucleic acid sequence having any one of SEQ ID NOS: 2, 4, 6, 8, 10, 12, 14, 16, or 18.

In some embodiments, the second portion comprises a nucleic acid sequence having at least 80% identity to any one of SEQ ID NOS: 35 and 60. In some embodiments, the second portion comprises a nucleic acid sequence having at least 85% identity to any one of SEQ ID NOS: 35 and 60.

In some embodiments, the second portion comprises a nucleic acid sequence having at least 90% identity to any one of SEQ ID NOS: 35 and 60. In some embodiments, the second portion comprises a nucleic acid sequence having at least 92% identity to any one of SEQ ID NOS: 35 and 60. In some embodiments, the second portion comprises a nucleic acid sequence having at least 95% identity to any one of SEQ ID NOS: 35 and 60. In some embodiments, the second portion comprises a nucleic acid sequence having at least 96% identity to any one of SEQ ID NOS: 35 and 60. In some embodiments, the second portion comprises a nucleic acid sequence having at least 97% identity to any one of SEQ ID NOS: 35 and 60. In some embodiments, the second portion comprises a nucleic acid sequence having at least 98% identity to any one of SEQ ID NOS: 35 and 60. In some embodiments, the second portion comprises a nucleic acid sequence having at least 99% identity to any one of SEQ ID NOS: 35 and 60. In some embodiments, the second portion comprises a nucleic acid sequence having any one of SEQ ID NOS: 35 and 60.

In some embodiments, the one or more therapeutic proteins are selected from the group consisting of ALPL, PCSK9, PCSK7, SerpinA1, ABCB4 (having at least 80%, at least 85%, at least 90%, at least 95%, at least 97%, at least 99% identity to any one of SEQ ID NOS: 64, 65, 66, or 69), ATP7B (having at least 80%, at least 85%, at least 90%, at least 95%, at least 97%, at least 99% identity to any one of SEQ ID NOS: 70 or 83), A1AT (having at least 80%, at least 85%, at least 90%, at least 95%, at least 97%, at least 99% identity to SEQ ID NO: 66), ABCB11, antiCD19-antiCD3 (having at least 80%, at least 85%, at least 90%, at least 95%, at least 97%, at least 99% identity to SEQ ID NO: 67), BDF8 (having at least 80%, at least 85%, at least 90%, at least 95%, at least 97%, at least 99% identity to SEQ ID NO: 68) and their variants thereof. In some embodiments, the one or more therapeutic proteins are alkaline phosphatase or a variant thereof.

In some embodiments, the non-viral DNA vector further comprises a S/MAR element. In some embodiments, the non-viral DNA vector further comprises an insulator element. In some embodiments, the expression cassette further comprises a polyadenylation site downstream of the one or more nucleic acid sequences encoding the one or more therapeutic proteins.

A fifth aspect of the present disclosure is an isolated, circular, non-viral DNA vector comprising: a first portion comprising an expression cassette including a nucleic acid sequence encoding a polypeptide having any one of Formulas (IA) to (IE), where the nucleic acid sequence encoding the polypeptide is operatively linked to a promoter; and a second portion capable of forming at least one cruciform structure, wherein the second portion comprises at least two inverted repeat sequences, and where the at least two inverted repeat sequences are separated by a non-repeating nucleotide sequence having at least 3 nucleotides;

[A]v-[B]-[C]w-[R]q-([D]x-[E]y)z (IA),

[A]-[B]-[C]-[R]q-([D]x-[E]y) (IB),

([A]-[B])-([D]x-[E]y)z IC),

([A]-[B])-([E]y) (ID), and

[A]-[B]-[R]q-([E]y) (IE)
wherein
A comprises an amino acid sequence encoding a secretion signal peptide;
B comprises an amino acid encoding an alkaline phosphatase;
C comprises an amino acid sequence encoding a GPI anchor;
R is -(Mo(Fc)Np)-, where M and N each independently include between 1 and 6 amino acids, where Fc is a Fc domain, and o and p are each independently 0, 1, or 2;
D comprises an amino acid sequence having between 4 and 6 amino acids, or is F(G)tF, where each F is the same amino acid, G is an amino acid sequence having 3, 4, or 5 amino acids, and t is an integer ranging from 2-5;
E comprises an amino acid sequence having between 1 and 8 amino acids;
q is 0 or 1;
v is 0 or 1;
w is 0 or 1;
x is 0 or an integer ranging from 1 to 6;
y is 0 or an integer ranging from 1 to 16; and
z is 0 or an integer ranging from 1 to 6;
In some embodiments, when v is 1, w is 0, q is 1, o is 1, p is 1, N is the diamino acid -D-I-M is the diamino acid -L-K-, [B] comprises SEQ ID NO: 11, Fc comprises SEQ ID NO: 130, and
x is 0, then [E]y does not comprise ten to sixteen contiguous aspartic acid residues.

In some embodiments, E comprises 3 amino acids. In some embodiments, E is -D-S-S-. In some embodiments, E is -D-S-S-, and y ranges from 1 to 16. In some embodiments, E is -D-S-S-, y is 6, z is 1, q is 0, and x is 0. In other embodiments, E is -D-S-S-, y is 6, z is 1, q is 0, and x is 2. In some embodiments, E is -D-S-S-, y is 6, z is 1, x is 2, and q is 1.

A sixth aspect of the present disclosure is an isolated, circular, non-viral DNA vector having a nucleic acid sequence having at least 80% identity to any one of SEQ ID NOS: 28-30, 38, 40-48, and 72-73, e.g., at least 85%, at least 90%, at least 91%, at least 92%, at least 93%, at least 94%, at least 95%, at least 96%, at least 97%, at least 98%, at least 99% identity to any one of SEQ ID NOS: 28-30, 38, 40-48, and 72-73. In some embodiments, the isolated, circular, non-viral DNA is provided in a pharmaceutically acceptable medium. In some embodiments, the isolated, circular, non-viral DNA is formulated with one or more lipid nanoparticles. In some embodiments, the isolated, circular, non-viral DNA is formulated with one or more polymer nanoparticles. In some embodiments, the isolated, circular, non-viral DNA is formulated with one or more proteo-lipid nanoparticles.

A seventh aspect of the present disclosure is an isolated, circular, non-viral DNA vector having a nucleic acid sequence having any one of SEQ ID NOS: 28-30, 38, 40-48, and 72-73. In some embodiments, the isolated, circular, non-viral DNA is provided in a pharmaceutically acceptable medium. In some embodiments, the isolated, circular, non-viral DNA is formulated with one or more lipid nanoparticles. In some embodiments, the isolated, circular, non-viral DNA is formulated with one or more polymer nanoparticles. In some embodiments, the isolated, circular, non-viral DNA is formulated with one or more proteo-lipid nanoparticles.

An eighth aspect of the present disclosure is a pharmaceutical composition comprising any one of the isolated, circular, non-viral DNA vectors of any one of the preceding embodiments and a pharmaceutically acceptable carrier or excipient. In some embodiments, the pharmaceutical composition is formulated with a lipid-based delivery vehicle. In some embodiments, the pharmaceutical composition is formulated as lipid nanoparticles. In some embodiments, the pharmaceutical composition is formulated as lipid nanocapsules. In some embodiments, the pharmaceutical composition is formulated with one or more polymers.

A ninth aspect of the present disclosure is a method of treating a patient in need of treatment thereof, comprising administering any of the pharmaceutical compositions described above. In some embodiments, the pharmaceutical composition is administered weekly. In some embodiments, the pharmaceutical composition is administered monthly. In some embodiments, the pharmaceutical composition is administered every two months. In some embodiments, the pharmaceutical composition is administered every six months. In some embodiments, the pharmaceutical composition is administered yearly. In some embodiments, the pharmaceutical composition is administered every two years. In some embodiments, the pharmaceutical composition is administered every several years.

A tenth aspect of the present disclosure is a method of treating hypophosphatasia, comprising administering a therapeutically effective amount of any of the isolated, circular, non-viral DNA vectors described above or a pharmaceutical composition comprising the same.

An eleventh aspect of the present disclosure is a method of treating, mitigating, or preventing a symptom of hypophosphatasia, comprising administering a therapeutically effective amount of any of the isolated, circular, non-viral DNA vectors described above or a pharmaceutical composition comprising the same.

A twelfth aspect of the present disclosure is directed to the use of any of the isolated, circular, non-viral DNA vectors described above (or a pharmaceutical composition comprising the same) for treating hypophosphatasia.

### BRIEF DESCRIPTION OF THE FIGURES

For a general understanding of the features of the disclosure, reference is made to the drawings. In the drawings, like reference numerals have been used throughout to identify identical elements.

FIG. 1A illustrates a non-viral DNA vector comprising (i) a first portion comprising an expression cassette including one or more nucleic acid sequences encoding one or more therapeutic proteins, where each of the one or more nucleic acid sequences encoding the one or more therapeutic proteins are operatively linked to a promoter; and (ii) a second portion capable of forming at least one cruciform structure, wherein the second portion comprises at least two inverted repeat sequences, wherein the at least two inverted repeat sequences are separated by a non-repeating nucleotide sequence having at least 3 nucleotides. Here, the first portion includes a nucleic acid encoding the human tissue non-specific alkaline phosphatase (TNALP), the nucleic acid under control of a promoter, including any of those described herein. In some embodiments, the cruciform structure comprises a Holliday junction.

FIGS. 1B and 1C illustrate the non-viral DNA vector of FIG. 1A and illustrate the formed at least one cruciform structure. The inverted DNA repeat elements that lead to the formation of the cruciform DNA structure may contain internal inverted DNA repeats as depicted in FIG. 1B. These smaller internal inverted DNA repeat elements may form additional secondary structure that stabilizes the formation of the larger cruciform structure. As depicted in FIG. 1B, the cruciform structure is formed between specific repeat elements, however, due to the nature of these repeats, the cruciform may be formed between other repeat elements, or between the inverted repeat sequences and the remainder of the non-viral DNA vector. In some embodiments, the inverted repeat elements may also be discontinuous, with regions of non-base paired or single-stranded DNA. One such region containing non-base paired or single-stranded DNA is in the region comprising the non-repeating nucleotide sequence having at least 3 nucleotides, which is internal to the cruciform structure (see FIG. 1A). Given the components of the non-viral DNA vectors of the present disclosure, it is believed that at least two inverted repeat sequences, each separated by a non-repeating nucleotide sequence having at least 3 nucleotides, facilitates the formation of a single cruciform structure having two long arms capped by a region of single-stranded DNA.

FIG. 1D illustrates the non-viral DNA vector of FIG. 1A, and illustrates the formed at least one cruciform structure, and further depicts secondary structure of the incorporated portion of the non-repeating nucleotide sequence having at least 3 nucleotides, which may be a portion of a bacterial origin of replication.

FIG. 1E depicts the predicted secondary structure of a bacterial origin of replication between two inverted terminal repeats. This single large cruciform structure contains numerous regions of single-stranded and non-repeat forming DNA in the region of the bacterial origin of replication.

FIGS. 2A, 2B, 2C, 2D, and 2E depict the predicted DNA secondary structure and calculated Gibbs free energy, normalized calculated Gibbs free energy per bp, melting temperature (Tm) of zero-CpG internal repeat sequences of circular, non-viral DNA vectors according to the present disclosure (SEQ ID NO: 29, 30, and 31). Based on this assessment, although the two long inverted repeat sequences contain smaller internal inverted repeats, when containing a portion of a bacterial origin of replication, internal secondary structure may not be formed within the two long inverted repeat sequences.

FIG. 3 provides three representative transmission electron microscope photos of circular, non-viral DNA vectors of the present disclosure which include at least two inverted repeat sequences separated by at least a portion of a bacterial origin of replication (M012—SEQ ID NO:10).

FIG. 4 illustrates resolution of circular, non-viral vector DNA reporter constructs containing cruciform structures of the present disclosure (P004—SEQ ID NO: 35; P006—SEQ ID NO: 14) delivered by polyethylenimine (PEI) and SM-102-based lipid nanoparticle (LNP) in 293 cells. The data suggests resolution of reporter constructs delivered with LNP and PEI started from day 6. The data further suggests that different inverted repeat sequences will have different resolution efficiencies.

FIG. 5 illustrates simultaneous green fluorescent protein (GFP) and red fluorescent protein (RFP) expression at day 3, and largely RFP expression at day 8, in non-dividing induced pluripotent stem cell (iPSC)-derived human hepatocytes transfected with a circular, non-viral DNA vector including an RFP/GFP reporter (SEQ ID NO: 32).

FIG. 6 compares the gene expression and durability of firefly luciferase from two different constructs, namely from a circular, non-viral DNA vector of the present disclosure (SEQ ID NO: 30) and from a plasmid (SEQ ID NO: 31) in post-mitotic human iPSC-derived hepatocytes. The data reveals that transgene expression from the circular, non-viral DNA vectors of the present disclosure (SEQ ID NO: 30) with the cruciform structure is much higher and more stable than the plasmid construct (SEQ ID NO: 31).

FIG. 7 compares the secretive ALP activity of human tissue non-specific alkaline phosphatase (TNALP) in post-mitotic iPSC-derived hepatocytes from three different constructs, namely from a circular, non-viral DNA vector of the present disclosure containing a cruciform structure comprised of a long inverted repeat containing a bacterial origin of replication (M012—SEQ ID NO: 28), from a circular, non-viral DNA vector of the present disclosure containing a cruciform structure comprised of a shorter inverted repeat not containing a bacterial origin (M013 SEQ ID NO: 29), and from a regular plasmid vector (P020—SEQ ID NO: 33), respectively. The data suggest significantly enhanced transgene (TNALP) expression from vectors comprised of a long-inverted repeat containing a bacterial origin of replication.

FIG. 8A illustrates that a circular, non-viral DNA vector encoding a luciferase reporter (M014—SEQ ID NO: 30) in accordance with the present disclosure showed persistent bioluminescent signal from mouse liver tissue harvested from week 1 to weeks 4 and 5 after dosing through hydrodynamic tail vein injection compared with the fast decay of control luciferase plasmid. FIG. 8B shows that DNA copy number results matched gene expression of two DNA vectors. The DNA copy number of the circular, non-viral DNA vector was maintained for one month in mouse liver tissue while the copy number of a control luciferase plasmid (P021—SEQ ID NO: 31) in mouse liver cells dropped significantly from week 1 to week 5 as shown in FIG. 8B.

FIG. 9 shows data from a single time point comparing between two different studies. The first study used a circular, non-viral DNA vector including a nucleic acid encoding TNALP; while the second study utilized mobilized human hematopoietic stem cells transduced with a lentiviral vector expressing TNALP. Similar plasma ALP activities were mediated in mice in the two different studies.

FIG. 10 illustrates the canonical mechanism of Holliday junction resolution (A) Antiparallel stacked-X Holliday junction with twofold symmetry. (B) Canonical Holliday junction resolvases are dimeric enzymes that induce structural changes to the junction on binding, causing the junction to unfold. Resolution occurs by the introduction of two coordinated and symmetrically related nicks in strands of like polarity at, or very near, the branchpoint. (C) Symmetrical resolution gives a pair of nicked DNA duplexes, each of which can be directly repaired by nick ligation, or homologous sequences can serve as templates for non-homologous end-joining (NHEJ) or homology-directed repair (HDR). Asterisks signify a given strand of DNA.

FIG. 11 illustrates the mechanism of resolution of the presently disclosed circular, non-viral DNA vector containing at least one cruciform structure (Holliday junction) (Form A) Once introduced into a eukaryotic cell, Holliday junction resolvases introduce two coordinated and symmetrically related nicks in strands of like polarity at, or very near, the branchpoint. Symmetrical resolution gives a nicked circular or linear DNA duplex, which can be directly repaired by nick ligation (yielding Form B), or homologous sequences can serve as templates for NHEJ or HDR (yielding Form C). It is further anticipated that this process could be repeated yielding higher molecular weight linear or circular forms.

FIGS. 12A and 12B illustrate the persistence of circular, non-viral DNA vectors of the present disclosure encoding a murine secreted embryonic alkaline phosphatase (SEAP) reporter (M027 and M032—SEQ ID NO: 74 and SEQ ID NO: 75) in a rodent model through hydrodynamic tail vein injection at 15 μg DNA per mouse per dose. High plasma SEAP activity in mouse plasma was maintained from day 1 to day 190 after double dosing through hydrodynamic tail vein injection (15 μg of DNA per mouse per dose). Further, DNA vector copy number per diploid cell analysis showed that the three constructs according to the present disclosure (M027—SEQ ID NO: 74; M032—SEQ ID NO: 75) were maintained for 183 days in mouse liver (FIG. 12C).

FIGS. 13A and 13B illustrate a comparison of two constructs according to the present disclosure, where the two constructs have two different cruciform structures, namely M012 (SEQ ID NO: 30) and M056 (SEQ ID NO: 73) in iPSC-derived hepatocytes at day 3. FIG. 13A shows that the cells transfected with the construct M056 (SEQ ID NO: 73) with the further optimized cruciform structure had much higher ALP secretion level in medium than those transfected with M012 (SEQ ID NO: 30) with both FuGENE and SM102-based lipid nanoparticle formulations. FIG. 13B shows that both constructs (M012—SEQ ID NO: 30 and M056—SEQ ID NO: 73), which have different structures, do not significant impact the viability of transfected cells.

FIGS. 14A, 14B and 14C illustrate the enhanced nuclear entry of constructs according to the present disclosure having a cruciform structure (M012—SEQ ID NO: 28; M013—SEQ ID NO: 29) as compared with DNA without a crucifom1 structure (M022—SEQ ID NO: 33) in post-mitotic non-dividing iPSC-derived hepatocytes after transfection. FIG. 14A shows a representative image in which DAPI staining (blue) represents the nucleus, green foci without green circle represents the constructs of the present disclosure in cytoplasm, green foci marked with green circle represents constructs of the present disclosure in the nucleus. The fluorescence-labeled circular, non-viral DNA vector including a nucleic acid encoding TNALP and also including dual inverted repeats (M012—SEQ ID NO: 28) showed highest number of foci per nucleus at day 3 and 6 as compared with (i) a circular, non-viral DNA vector including single inverted repeats without intervening heterologous sequences (DD-ITR construct) (M013—SEQ ID NO: 29), and (ii) a control and without cruciform structure at all (M022—SEQ ID NO: 33) (FIG. 14B). There is a correlation between the number of foci per nucleus and measured ALP activity in the cell culture medium (FIG. 14C).

FIGS. 15A and 15B illustrate that constructs of the present disclosure including firefly luciferase DNA (SEQ ID NO: 73) with improved cruciform structure led to a much higher luciferase activity than control DNA without cruciform structure (SEQ ID NO: 74) across species both in human and cynomolgus monkey hepatocytes. Both the constructs according to the present disclosure with luciferase DNA (SEQ ID NO: 73) and control luciferase DNA without cruciform structure (SEQ ID NO: 74) were transfected to hepatocytes with SM102-based lipid nanoparticle formulation.

FIG. 16 illustrates that a construct according to the present disclosure with luciferase DNA (SEQ ID NO: 73) with improved cruciform structure also led to a much higher luciferase activity in mouse than control DNA without a cruciform structure (SEQ ID NO: 74). A circular, non-viral DNA vector including a firefly luciferase reporter (SEQ ID NO: 73) and a control DNA without cruciform structure (SEQ ID NO: 74) were dosed to mice through hydrodynamic tail vein injection (15 μg per mouse) and mouse liver tissues were harvested at day 7 and 14 before luciferase activity assay was carried out.

FIG. 17 illustrates the great durability of constructs according to the present disclosure (SEQ ID NO: 43) as compared with mRNA. Constructs according to the present disclosure (SEQ ID NO: 43) and chemically modified mRNA encoding cynomolgus monkey soluble TNALP transgene were transfected via SM102-based lipid nanoparticle formulation to primary human hepatocytes. ALP activity from constructs according to the present disclosure in culture medium increased from day 1 to 3, then was maintained from day 3 to day 6. At the same time, ALP activity of mRNA-transfected cells decreased from day 1 to 5 and diminished at day 6.

FIG. 18 illustrates that co-localization of PARP1 (purple color) and constructs according to the present disclosure (green color) (M012—SEQ ID NO: 28) in the cytoplasm of 293 cells 24 hours post-transfection. PARP1 is a well-known first responder that detects DNA damage and then facilitates genomic DNA repair pathway. The colocalization data suggests that PARP1 might be one of the factors involved in the innate immune evasion, resolution, nuclear entry, recombination, and nuclear retention of constructs according to the present disclosure.

FIGS. 19A, 19B and 19C illustrate immunomodulatory effects of circular, non-viral constructs according to the present disclosure. FIG. 19B shows that a human TNALP DNA construct according to the present disclosure (M012—SEQ ID NO: 28) with optimized cruciform structure led to a much higher ALP activity, more durable transgene expression, and no immune response (as shown by the ability to re-dose) in mouse as compared with a non-viral human TNALP DNA vector with an early version of a cruciform structure including single inverted repeats without intervening heterologous sequences (DD-ITR construct) (M013—SEQ ID NO: 29) (FIG. 19A). FIG. 19C illustrates that optimized cynomolgus monkey TNALP DNA construct according to the present disclosure (M026—SEQ ID NO: 43) with improved cruciform structure further increased potency, durability of the transgene expression while maintaining the ability to re-dose as compared with the similar construct without gene cassette optimization. Non-viral DNA constructs were administered repeatedly at 3-week intervals through hydrodynamic tail vein injection (15 μg per mouse) and plasma samples were collected weekly for assessment of ALP activity.

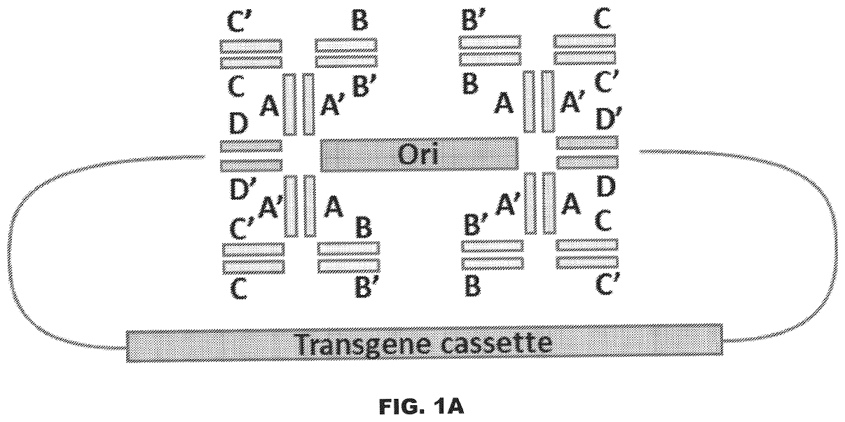

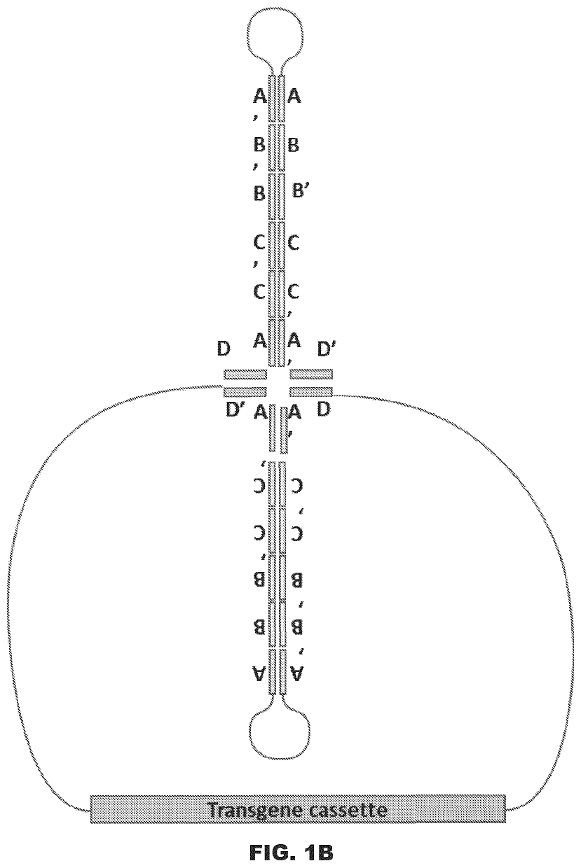

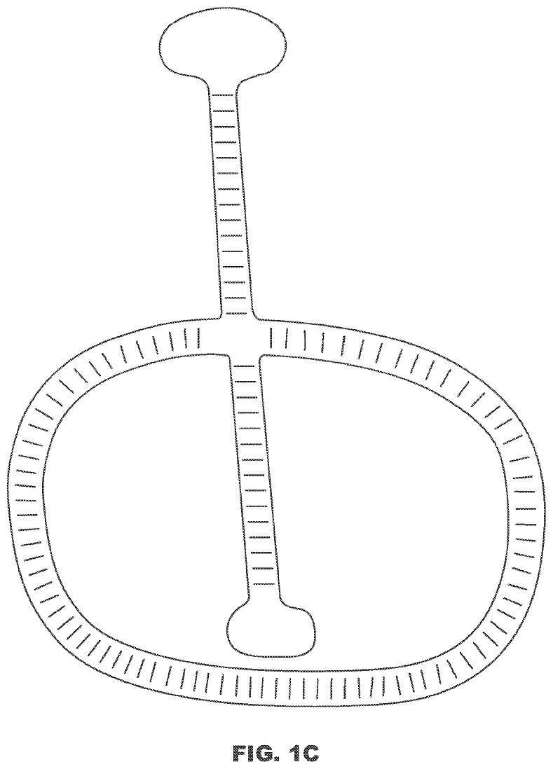

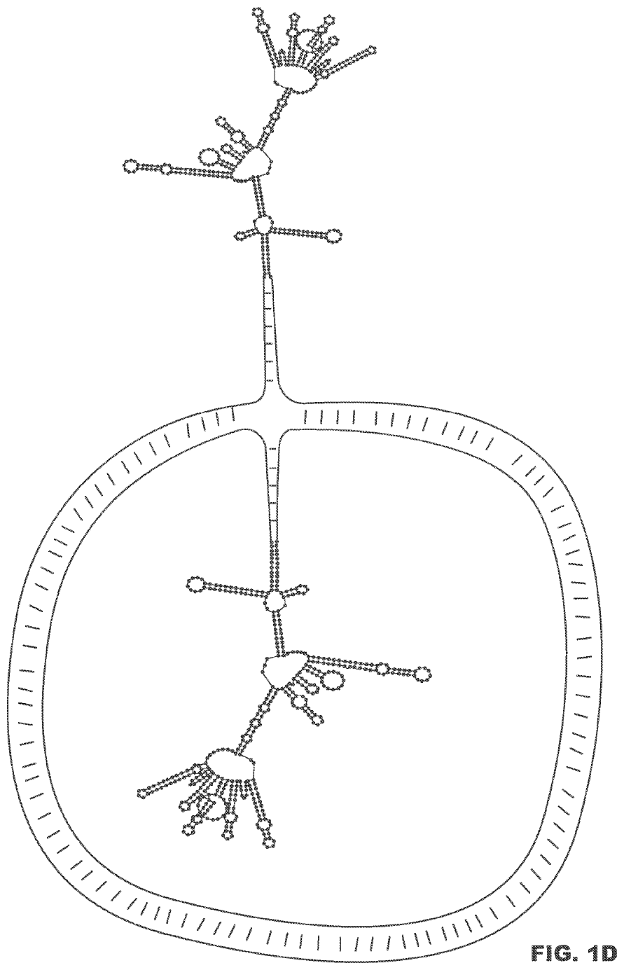

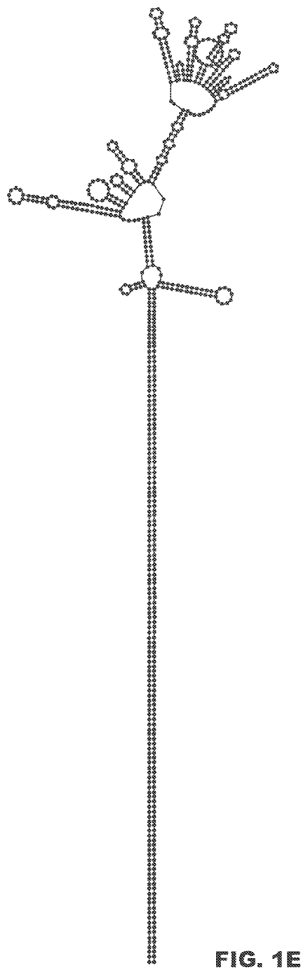

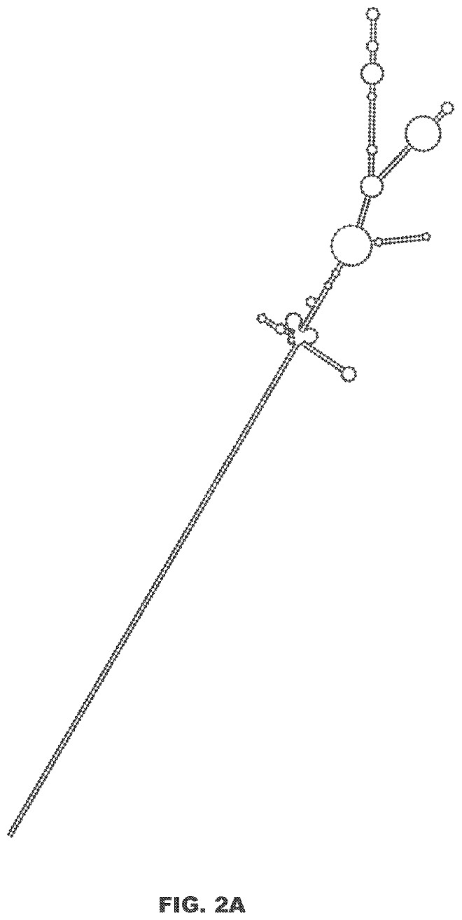

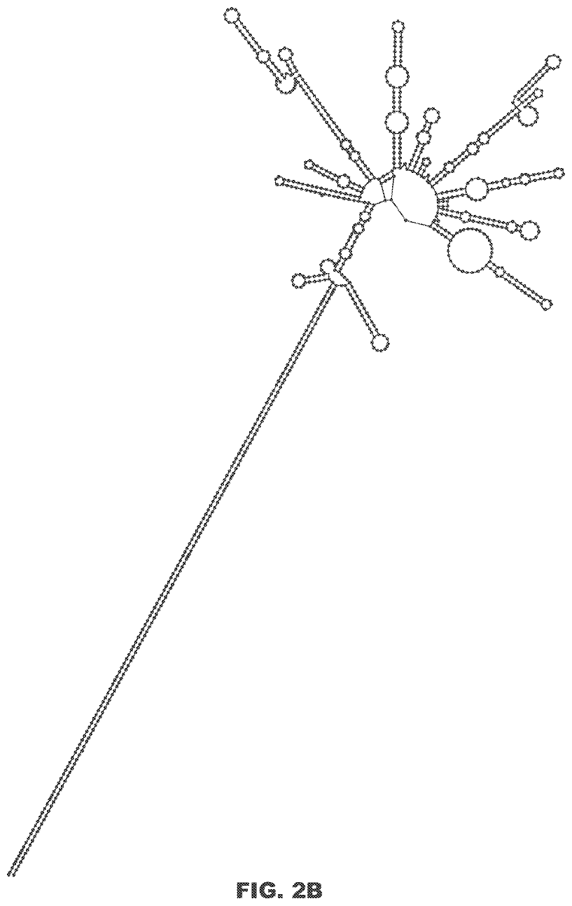

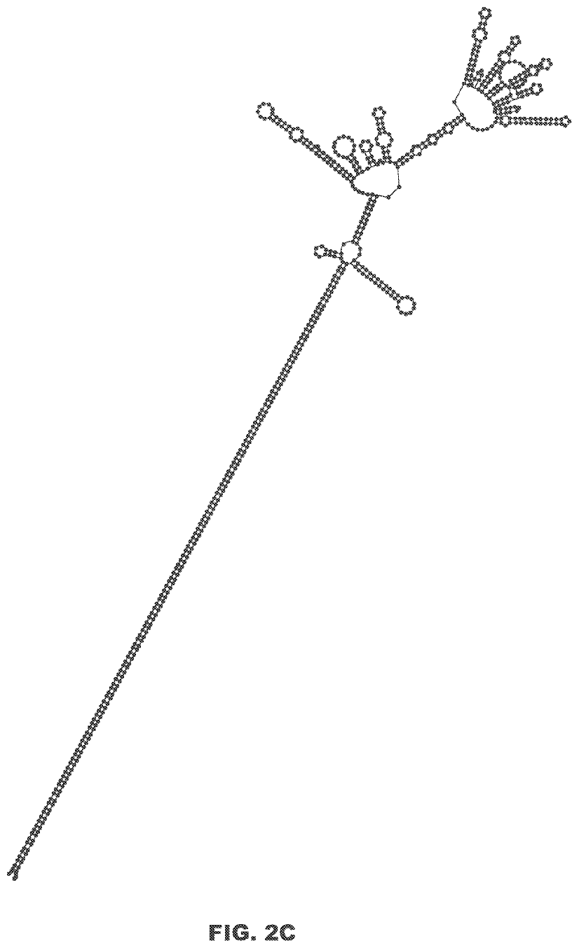

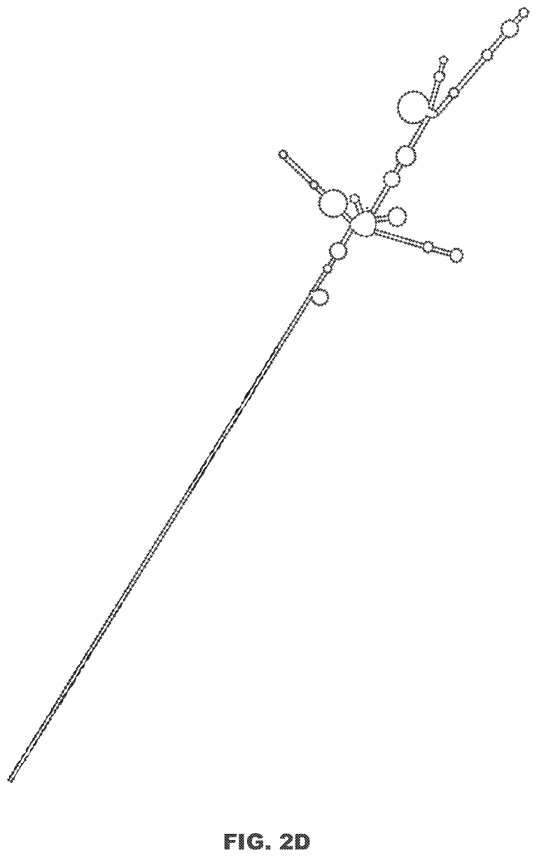

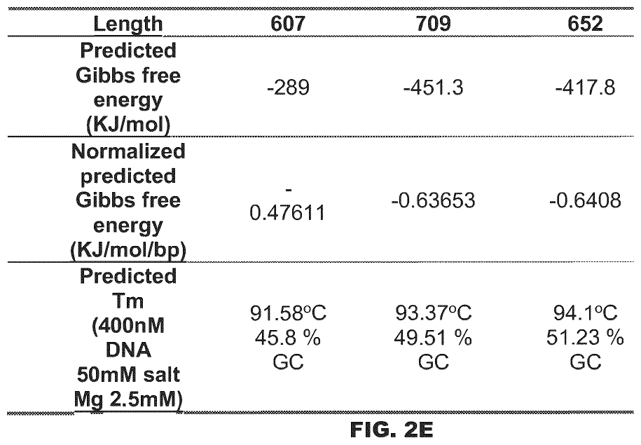

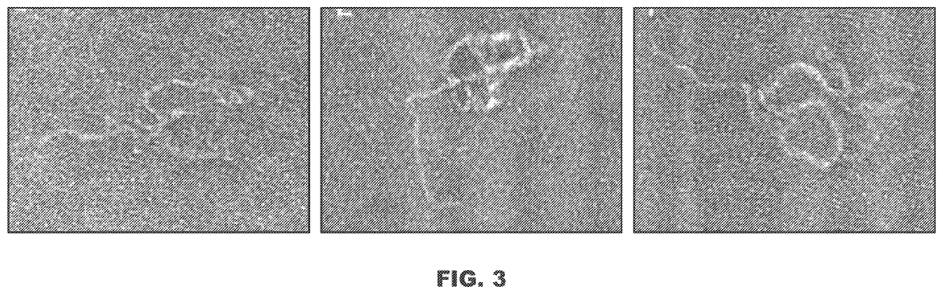

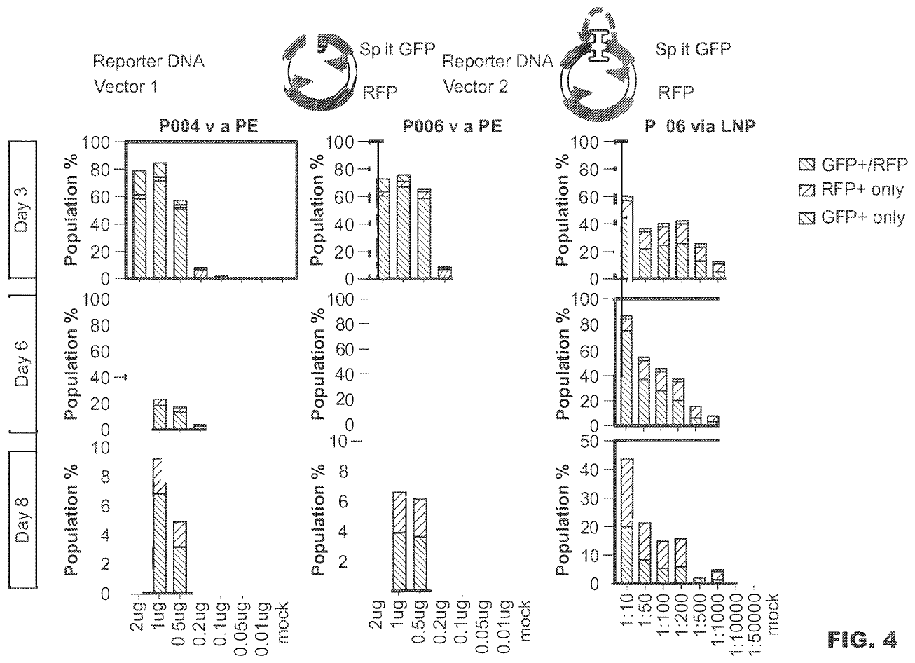

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one, of a number or list of elements, and, optionally, additional unlisted items. Only terms clearly indicated to the contrary, such as “only one of’ or “exactly one of,” or, when used in the claims, “consisting of,” will refer to the inclusion of exactly one element of a number or list of elements. In general, the term “or” as used herein shall only be interpreted as indicating exclusive alternatives (e.g., “one or the other but not both”) when preceded by terms of exclusivity, such as “either,” “one of,” “only one of’ or “exactly one of.” “Consisting essentially of,” when used in the claims, shall have its ordinary meaning as used in the field of patent law.

As used herein, the terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b, and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “about” refers to a measurable value such as an amount of the length of a nucleotide sequence, a polynucleotide or polypeptide sequence, dose, time, temperature, and the like, is meant to encompass variations of 20%, 10%, 5%, 1%, 0.5%, or even 0.1% of the specified amount.

As used herein, the term “Fc” refers to a human IgG Fc domain. Subtypes of IgG such as IgG1, IgG2, IgG3, and IgG4 are all being contemplated for usage as Fc domains.

As used herein, the term “heterologous gene” as used herein refers to a nucleotide sequence not naturally associated with a host animal into which it is introduced, including for example, exon coding sequences from a human gene introduced, as a chimeric heterologous gene, into a host nematode.

As used herein, the term “host cell” refers to any cell type that is susceptible to transformation, transfection, transduction, or the like with a nucleic acid construct or expression vector comprising a polynucleotide of the present disclosure. In some embodiments, the term “host cell” encompasses any progeny of a parent cell that is not identical to the parent cell due to mutations that occur during replication. Host cells may include packaging cells, producer cells, and cells infected with viral vectors. In particular embodiments, host cells infected with viral vector of the present disclosure are administered to a subject in need of therapy. In some embodiments, host cells are transduced ex vivo. In other embodiments, host cells are transduced in vivo.

As used herein, the terms “hypophosphatasia” and “HPP” refer to a rare, heritable skeletal disorder caused by, e.g., one or more loss-of-function mutations in the ALPL (alkaline phosphatase, liver/bone/kidney) gene, which encodes tissue-nonspecific alkaline phosphatase (TNALP). HPP can be further characterized as, e.g., infantile HPP or perinatal HPP (e.g., benign perinatal HPP or lethal perinatal HPP). For instance, “infantile HPP” describes a patient having HPP that is about three years of age or younger, whereas “perinatal HPP” describes a patient having HPP immediately before or after birth (e.g., one to four weeks after birth). The age of onset of HPP, such as when the subject exhibits symptoms of HPP, can also be categorized as, e.g., perinatal-onset HPP and infantile-onset HPP. Patients with HPP can exhibit symptoms of HPP including, but not limited to, skeletal deformity, hypotonia, mobility impairments, gait disturbance, bone deformity, joint pain, bone pain, bone fracture, muscle weakness, muscle pain, rickets (e.g., defects in growth plate cartilage), premature loss of deciduous teeth, incomplete bone mineralization, elevated blood and/or urine levels of phosphoethanolamine (PEA), PPi, pyridoxal 5′-phosphate (PLP), hypomineralization, rachitic ribs, hypercalciuria, short stature, HPP-related seizure, inadequate weight gain, craniosynostosis, and/or calcium pyrophosphate dihydrate crystal deposition (CPPD) in joints leading to, e.g., chondrocalcinosis and premature death. Symptoms of HPP can also include TBM and symptoms of TBM, such as cardio-respiratory arrest, tracheostomy, cardiac arrest, respiratory distress, sputum retention, wheezing, coughing, anoxic spells, cyanosis, bradycardia, tachyarrhythmia, spontaneous hyperextension of the neck, prolonged expiratory breathing phase, failure to thrive, sternal retractions, substernal retractions, intercostal retractions, intermittent or continuous dyspnea, and recurrent bronchitis or pneumonia.

As used herein, the term “nucleic acid” refers to polynucleotides such as DNA or RNA. Nucleic acids can be single-stranded, partly, or completely, double-stranded, and in some cases partly or completely triple-stranded. Nucleic acids include genomic DNA, cDNA, mRNA, etc. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. The term “nucleic acid sequence” as used herein can refer to the nucleic acid material itself and is not restricted to the sequence information (i.e., the succession of letters chosen among the five base letters A, G, C, T, or U) that biochemically characterizes a specific nucleic acid, e.g., a DNA or RNA molecule. A nucleic acid sequence is presented in the 5′ to 3′ direction unless otherwise indicated. The term “nucleic acid segment” is used herein to refer to a nucleic acid sequence that is a portion of a longer nucleic acid sequence.

As used herein, the terms “operably linked” or “operably associated” refer to a functional relationship between two nucleic acids, wherein the expression, activity, localization, etc., of one of the sequences is controlled by, directed by, regulated by, modulated by, etc., the other nucleic acid. The two nucleic acids are said to be operably linked or operably associated or in operable association. “Operably linked” or “operably associated” can also refers to a relationship between two polypeptides wherein the expression of one of the polypeptides is controlled by, directed by, regulated by, modulated by, etc., the other polypeptide. For example, transcription of a nucleic acid is directed by an operably linked promoter; post-transcriptional processing of a nucleic acid is directed by an operably linked processing sequence; translation of a nucleic acid is directed by an operably linked translational regulatory sequence such as a translation initiation sequence; transport, stability, or localization of a nucleic acid or polypeptide is directed by an operably linked transport or localization sequence such as a secretion signal sequence; and post-translational processing of a polypeptide is directed by an operably linked processing sequence. Typically, a first nucleic acid sequence that is operably linked to a second nucleic acid sequence, or a first polypeptide that is operatively linked to a second polypeptide, is covalently linked, either directly or indirectly, to such a sequence, although any effective three-dimensional association is acceptable. One of ordinary skill in the art will appreciate that multiple nucleic acids, or multiple polypeptides, may be operably linked or associated with one another.

As used herein, the terms “pharmaceutically acceptable excipient,” “carrier,” or “diluent” refer to pharmaceutical components which do not alter the therapeutic properties of an active agent with which it is administered. One exemplary pharmaceutically acceptable carrier substance is physiological saline. For instance, the pharmaceutically acceptable carrier can include sodium chloride (e.g., 150 mM sodium chloride) and sodium phosphate (e.g., 25 mM sodium phosphate). Other physiologically acceptable excipients, carriers, and diluents, and their formulations, are known to those skilled in the art and described, e.g., in Remington: The Science and Practice of Pharmacy (22nd Ed), Allen (2012). For instance, a pharmaceutically acceptable excipient, carrier, or diluent can include dibasic sodium phosphate, heptahydrate; monobasic sodium phosphate, monohydrate; and sodium chloride at a pH between 7.2 and 7.6.

As used herein, the term “pharmaceutical composition” it is meant a composition containing an active agent as described herein, formulated with at least one pharmaceutically acceptable excipient, carrier, or diluent. The pharmaceutical composition can be manufactured or sold with the approval of a governmental regulatory agency as part of a therapeutic regimen for the treatment or prevention of a disease or event in a patient (e.g., an infant with HPP, such as an infant having perinatal-onset HPP, or an infant having infantile-onset HPP, or juvenile-onset HPP, or a patient having childhood-onset HPP). Pharmaceutical compositions can be formulated, for example, for subcutaneous administration, intravenous administration (e.g., as a sterile solution free of particulate emboli and in a solvent system suitable for intravenous use), for oral administration (e.g., a tablet, capsule, caplet, gelcap, or syrup), or any other formulation described herein, e.g., in unit dosage form.

As used herein, the term “polypeptide,” “polypeptide fragment,” “peptide” and “protein” are used interchangeably, unless specified to the contrary, and according to conventional meaning, i.e., as a sequence of amino acids. Polypeptides are not limited to a specific length, e.g., they may comprise a full-length protein sequence or a fragment of a full-length protein, and may include post-translational modifications of the polypeptide, for example, glycosylations, acetylations, phosphorylations and the like, as well as other modifications known in the art, both naturally occurring and non-naturally occurring. In various embodiments, the polypeptides contemplated herein comprise a signal (or leader) sequence at the N-terminal end of the protein, which co-translationally or post-translationally directs transfer of the protein. Polypeptides can be prepared using any of a variety of well-known recombinant and/or synthetic techniques. In some embodiments, the polypeptides contemplated herein encompass alkaline phosphatases, or sequences that have deletions from, additions to, and/or substitutions of one or more amino acid of a CAR as disclosed herein.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

As used herein, the term “promoter,” as used herein, refers to a DNA sequence that determines the site of transcription initiation for an RNA polymerase. Promoter sequences comprise motifs which are recognized and bound by polypeptides, i.e., transcription factors. The said transcription factors shall upon binding recruit RNA polymerases II, preferably, RNA polymerase I, II or III, more preferably, RNA polymerase II or III, and most preferably, RNA polymerase II. Thereby will be initiated the expression of a nucleic acid operatively linked to the transcription control sequence. It is to be understood that dependent on the type of nucleic acid to be expressed, expression as meant herein may comprise transcription of DNA sequences into RNA polynucleotides (as suitable for, e.g., anti-sense approaches, RNAi approaches or ribozyme approaches) or may comprise transcription of DNA sequences into RNA polynucleotides followed by translation of the said RNA polynucleotides into polypeptides (as suitable for, e.g., gene expression and recombinant polypeptide production approaches). In order to govern expression of a nucleic acid sequence, the transcription control sequence may be located immediately adjacent to the nucleic acid to be expressed, i.e., physically linked to the said nucleic acid at its 5′ end. Alternatively, it may be located in physical proximity. In the latter case, however, the sequence must be located so as to allow functional interaction with the nucleic acid to be expressed.

As used herein, the terms “regulatory sequence” or “regulatory element” refer to a nucleic acid sequence that regulates one or more steps in the expression (particularly transcription, but in some cases other events such as splicing or other processing) of nucleic acid sequence(s) with which it is operatively linked. The term includes promoters, enhancers, and other transcriptional control elements that direct or enhance transcription of an operatively linked nucleic acid. Regulatory sequences may direct constitutive expression (e.g., expression in most or all cell types under typical physiological conditions in culture or in an organism), cell type specific, lineage specific, or tissue specific expression, and/or regulatable (inducible or repressible) expression. For example, expression may be induced or repressed by the presence or addition of an inducing agent such as a hormone or other small molecule, by an increase in temperature, etc. Non-limiting examples of cell type, lineage, or tissue specific promoters appropriate for use in mammalian cells include lymphoid-specific promoters (see, for example, Calame et al., Adv. Immunol. 43:235, 1988) such as promoters of T cell receptors (see, e.g., Winoto et al., EMBO J. 8:729, 1989) and immunoglobulins (see, for example, Banerji et al., Cell 33:729, 1983; Queen et al., Cell 33:741, 1983), and neuron-specific promoters (e.g., the neurofilament promoter; Byrne et al., Proc. Natl. Acad. Sci. USA 86:5473, 1989). Developmentally regulated promoters include hox promoters (see, e.g., Kessel et al., Science 249:374, 1990) and the a-fetoprotein promoter (Campes et al., Genes Dev. 3:537, 1989). Some regulatory elements may inhibit or decrease expression of an operatively linked nucleic acid. Such regulatory elements may be referred to as “negative regulatory elements.” A regulatory element whose activity can be induced or repressed by exposure to an inducing or repressing agent and/or by altering environmental conditions is referred to herein as a “regulatable” element.

As used herein, the term “signal peptide” refers to a short peptide (about 5 to about 30 amino acids long) at the N-terminus of a polypeptide that directs a polypeptide towards the secretory pathway (e.g., the extracellular space). In some embodiments, the signal peptide is typically cleaved during secretion of the polypeptide. In some embodiments, the signal sequence may direct the polypeptide to an intracellular compartment or organelle. In some embodiments, a signal sequence may be identified by homology, or biological activity, to a peptide with the known function of targeting a polypeptide to a particular region of the cell.

As used herein, the term “subject” refers to any animal subject including laboratory animals (e.g., primates, rats, mice), livestock (e.g., cows, sheep, goats, pigs, turkeys, chickens), household pets (e.g., dogs, cats, rodents, etc.), and humans.

As used herein, the term “therapeutically effective amount” refers to a virus or transduced therapeutic cell may vary according to factors such as the disease state, age, sex, and weight of the individual, and the ability of the stem and progenitor cells to elicit a desired response in the individual. In some embodiments, a therapeutically effective amount is also one in which any toxic or detrimental effects of the virus or transduced therapeutic cells are outweighed by the therapeutically beneficial effects. In some embodiments, the term “therapeutically effective amount” includes an amount that is effective to “treat” a subject (e.g., a patient).

As used herein, the terms “treatment” or “treating” refer to any beneficial or desirable effect on the symptoms or pathology of a disease or pathological condition and may include even minimal reductions in one or more measurable markers of the disease or condition being treated. In some embodiments, the treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, the terms “variants” or “variant” refer to a nucleic acid or polypeptide differing from a reference nucleic acid or polypeptide but retaining essential properties thereof. Generally, variants are overall closely similar, and, in many regions, identical to the reference nucleic acid or polypeptide. Thus, “variant” forms of a transcription factor are overall closely similar, and capable of binding DNA and activate gene transcription.

As used herein, the term “vector” refers to a nucleic acid molecule capable transferring or transporting another nucleic acid molecule. In some embodiments, the transferred nucleic acid is generally linked to, e.g., inserted into, the vector nucleic acid molecule. In some embodiments, a vector may include sequences that direct autonomous replication in a cell or may include sequences sufficient to allow integration into host cell DNA. Useful vectors include, for example, plasmids (typically DNA plasmids, but RNA plasmids are also of use), cosmids, and viral vectors. As will be evident to one of skill in the art, the term “viral vector” is widely used refer either to a nucleic acid molecule (e.g., a plasmid) that includes virus-derived nucleic acid elements that typically facilitate transfer of the nucleic acid molecule or integration into the genome of a cell or to a viral particle that mediates nucleic acid transfer. In some embodiments, the viral particles will typically include various viral components and sometimes also host cell components in addition to nucleic acid(s).

Circular, Non-Viral DNA Vectors The present disclosure is directed to circular, non-viral DNA vectors, such as circular non-viral DNA vectors including at least two inverted repeat sequences, where the at least two inverted repeat sequences are separated by a non-repeated nucleotide sequence which is not part of the at least two inverted repeat sequences. In some embodiments, the non-repeated nucleotide sequence comprises at least 3 nucleotides. In other embodiments, the non-repeated nucleotide sequence comprises at least 4 nucleotides. In other embodiments, the non-repeated nucleotide sequence comprises at least 5 nucleotides. In other embodiments, the non-repeated nucleotide sequence comprises at least 7 nucleotides. In other embodiments, the non-repeated nucleotide sequence comprises at least 10 nucleotides. In other embodiments, the non-repeated nucleotide sequence comprises at least 15 nucleotides. In other embodiments, the non-repeated nucleotide sequence comprises at least 20 nucleotides. Examples of suitable non-repeated nucleotide sequences include, but are not limited to, TTG, AAT, TAA, TAG, AGTT, AGTA, TAAA, TCAA, GAGTA, AAGTGCA, AGTACAAATTG, and ACCTTAGAGGCTA.

In some embodiments, the non-repeated nucleotide sequence comprises a minimal bacterial origin of replication. In other embodiments, the non-repeated nucleotide sequence comprises a heterologous gene or a portion of a heterologous gene, e.g., a gene encoding a bacterial suppressor tRNA, an RNAi repressor, or an antisense RNA. In other embodiments, the non-repeated nucleotide sequence comprises a bacterial operator sequence, e.g., the lac operator or tet operator.

In some embodiments, the circular, non-viral DNA vectors are substantially devoid of CpG sequences. In some embodiments, the circular, non-viral DNA vectors comprise less than about 500 CpG per vector, less than about 450 CpG per vector, less than about 400 CpG per vector, less than about 350 CpG per vector, less than about 300 CpG per vector, less than about 250 CpG per vector, less than about 200 CpG per vector, less than about 150 CpG per vector, less than about 100 CpG per vector, less than about 75 CpG per vector, less than about 50 CpG per vector, less than about 25 CpG per vector, less than about 20 CpG per vector, less than about 15 CpG per vector, less than about 10 CpG per vector, etc.

In some embodiments, the circular, non-viral DNA vectors lack a drug resistance gene. In some embodiments, the circular, non-viral DNA vectors include the two inverted repeat sequences separated by the non-repeated nucleotide sequence; where the vector further includes at least a portion of a bacterial origin of replication, and where the at least the portion of the bacterial origin of replication is not included within either of the two inverted repeat sequences or in the non-repeated nucleotide sequence. In some embodiments, the at least the portion of the bacterial origin of replication is located adjacent to the heterologous gene. In some embodiments, the circular, non-viral DNA vector includes one or more recombination sites, e.g., LoxP sites, FRT sites, attB and attP sites or their product sites attL or attR, or alternative recombination target sites derived from these sites, e.g., Lox7, Lox511 or Lox66 sites.

In some embodiments, the circular, non-viral DNA vectors are substantially double stranded. In other embodiments, the circular, non-viral DNA vectors are substantially supercoiled. In other embodiments, the substantially supercoiled non-viral DNA vectors comprise one or more regions of negative supercoiling.

In some embodiments, the circular, non-viral DNA vectors are non-immunogenic (e.g., have a reduced likelihood of triggering an immune response as compared with other non-viral DNA vectors, or viral vectors, such as lentiviral vectors, adenoviral vectors, and adeno-associated virus (AAV) vectors). It is also believed that the circular, non-viral DNA vectors of the present disclosure are amenable to repeat dosing, as described further herein. In addition, a reduction in immune-stimulating CpG content facilitates redosing, which is believed to be critical in pediatric patients.

In some embodiments, the circular, non-viral DNA vectors of the present disclosure facilitate persistent in vivo expression of one or more heterologous genes encoding one or more therapeutic proteins. Without wishing to be bound by any particular theory, it is believed that the non-viral DNA vectors of the present disclosure act like an endogenous gene in the nucleus for enhanced persistence of gene expression. In some embodiments, the circular, non-viral DNA vectors persist for a period of at least about 4 weeks after in vivo administration. In other embodiments, the circular, non-viral DNA vectors persist for a period of at least about 6 weeks after in vivo administration. In other embodiments, the circular, non-viral DNA vectors persist for a period of at least about 8 weeks after in vivo administration. In other embodiments, the circular, non-viral DNA vectors persist for a period of at least about 10 weeks after in vivo administration. In other embodiments, the circular, non-viral DNA vectors persist for a period of at least about 12 weeks after in vivo administration. In other embodiments, the circular, non-viral DNA vectors persist for a period of at least about 14 weeks after in vivo administration. In other embodiments, the circular, non-viral DNA vectors persist for a period of at least about 16 weeks after in vivo administration. In other embodiments, the circular, non-viral DNA vectors persist for a period of at least about 20 weeks after in vivo administration. In other embodiments, the circular, non-viral DNA vectors persist for a period of at least about 24 weeks after in vivo administration. In other embodiments, the circular, non-viral DNA vectors persist for a period of at least about 36 weeks after in vivo administration. In other embodiments, the circular, non-viral DNA vectors persist for a period of at least about 48 weeks after in vivo administration. In other embodiments, the circular, non-viral DNA vectors persist for a period of at least about 1 year after in vivo administration. In other embodiments, the circular, non-viral DNA vectors persist for a period of at least about 2 years after in vivo administration. In other embodiments, the circular, non-viral DNA vectors persist for a period of at least about 4 years after in vivo administration.

It is believed that the transgenes carried by the circular, non-viral DNA vectors of the present disclosure are taken up by cells and expressed at levels similar to transgene expression from viral vectors; and persist for periods similar to those of non-integrating viral vectors. Without wishing to be bound by any particular theory, it is believed that the circular, non-viral DNA vectors of the present disclosure act similar to some non-integrating viral vectors by assuming molecular forms that allow for persistent gene expression. Such similarities may be related to chromatin factors that both the circular, non-viral DNA vectors of the present disclosure and viral vectors interact with or related to chromatin structures assumed by both viral vectors and the non-viral DNA vectors of the present disclosure. Chromatin is a term that describes structures that organize DNA within the nucleus of eukaryotic cells. In its simplest conception, chromatin is composed of histone proteins that form nucleosomes on DNA. However, the spacing and modifications of these histones is complex and non-random and provide structural and signaling capacity to the protein scaffold surrounding genes or structural elements. The role of chromatin in virus biology depends largely on the type of virus, but for all viruses that transverse the nucleus, interactions with chromatin is unavoidable. The importance of chromatin dynamics in the regulation of essential viral-vector processes, including entry, gene expression, and persistence is beginning to be understood. The circular, non-viral DNA vectors of the present disclosure are thought to utilize a variety of aspects of chromatin dynamics in a manner akin to viral vectors. Such attributes could include chromatin structures that promote interaction with the nuclear matrix, structures that regulate epigenetic factors influencing gene expression, structures that mediate nuclear organization, or structures that promote active transcriptionally active chromatin status among others.

In some embodiments, the circular, non-viral DNA vector comprises (i) a first portion comprising an expression cassette including one or more nucleic acid sequences encoding one or more therapeutic proteins, where each of the one or more nucleic acid sequences encoding the one or more therapeutic proteins are operatively linked to a promoter; and (ii) a second portion capable of forming at least one cruciform structure, wherein the second portion comprises at least two inverted repeat sequences, and where the at least two inverted repeat sequences are separated by a non-repeating nucleotide sequence having at least 3 nucleotides. In some embodiments, the non-repeating nucleotide sequence has at least 5 nucleotides. In some embodiments, the non-repeating nucleotide sequence has at least 10 nucleotides. In some embodiments, the non-repeating nucleotide sequence has at least 15 nucleotides.

In some embodiments, the second portion does not include a bacterial origin or replication or any portion thereof. In other embodiments, the non-repeating nucleotide sequence does not comprise a bacterial origin or replication or any portion thereof. In other embodiments, the circular, non-viral DNA vector includes a bacterial origin of replication or a portion thereof, but the bacterial origin of replication or the portion thereof is not located within the second portion. In yet other embodiments, at least a portion of a bacterial origin of replication is included in the first portion or within a third portion adjacent to either the first or second portions, but not within the second portion.

In some embodiments, the cruciform structure comprises a Holliday junction.

In some embodiments, each of the inverted repeat sequences comprise or are derived from nucleic acid sequences present within AAV serotypes (including any of those disclosed herein).

In some embodiments, the non-repeating nucleotide sequence is single stranded, while the remainder of the second portion is double stranded. In some embodiments, the non-viral DNA vectors are substantially devoid of CpG sequences. In some embodiments, the non-viral DNA vectors comprise less than about 500 CpG per vector, less than about 450 CpG per vector, less than about 400 CpG per vector, less than about 350 CpG per vector, less than about 300 CpG per vector, less than about 250 CpG per vector, less than about 200 CpG per vector, less than about 150 CpG per vector, less than about 100 CpG per vector, less than about 75 CpG per vector, less than about 50 CpG per vector, less than about 25 CpG per vector, less than about 20 CpG per vector, less than about 15 CpG per vector, less than about 10 CpG per vector, etc.

In other embodiments, the circular, non-viral DNA vector comprises: (i) a first portion comprising an expression cassette including one or more nucleic acid sequences encoding one or more therapeutic proteins, where each of the one or more nucleic acid sequences encoding the one or more therapeutic proteins are operatively linked to a promoter; and (ii) a second portion capable of forming at least one cruciform structure, wherein the second portion has the Formula X—Y—X′, where X and X′ are each inverted repeat sequences, and where Y is not repeated and comprises a nucleotide having at least 3 nucleotides, e.g., at least 5 nucleotides, at least 10 nucleotides, at least 15 nucleotides, etc. In some embodiments, the second portion does not include a bacterial origin or replication or any portion thereof. In other embodiments, the circular, non-viral DNA vector includes a bacterial origin of replication or a portion thereof, but the bacterial origin of replication or the portion thereof is not located within the second portion.

In some embodiments, the inverted repeat sequences (X and X′) are derived from nucleic acid sequences present within AAV genomes. In some embodiments, the non-viral DNA vectors are substantially devoid of CpG sequences. In some embodiments, the non-viral DNA vectors comprise less than about 500 CpG per vector, less than about 450 CpG per vector, less than about 400 CpG per vector, less than about 350 CpG per vector, less than about 300 CpG per vector, less than about 250 CpG per vector, less than about 200 CpG per vector, less than about 150 CpG per vector, less than about 100 CpG per vector, less than about 75 CpG per vector, less than about 50 CpG per vector, less than about 25 CpG per vector, less than about 20 CpG per vector, less than about 15 CpG per vector, less than about 10 CpG per vector, etc.

In yet other embodiments, the non-viral DNA vector comprises the following elements operatively linked in a 5′ to a 3′ direction: (i) a first repeat sequence; (ii) a non-repeating nucleotide sequence having at least 3 nucleotides; (iii) a second repeat sequence; and (iv) an expression cassette. In some embodiments, the non-repeating nucleotide sequence has at least 5 nucleotides. In some embodiments, the non-repeating nucleotide sequence has at least 10 nucleotides. In some embodiments, the non-repeating nucleotide sequence has at least 15 nucleotides.

In some embodiments, the non-repeating nucleotide sequence does not comprise a bacterial origin or replication or any portion thereof. In other embodiments, the circular, non-viral DNA vector includes a bacterial origin of replication or a portion thereof, but the bacterial origin of replication or the portion thereof is not part of or included within the non-repeating nucleotide sequence. In some embodiments, the first and second repeat sequences comprise or are derived from nucleic acid sequences present within AAV genomes (including any of those disclosed herein).

In some embodiments, the expression cassette includes one or more nucleic acid sequences encoding one or more therapeutic proteins, where each of the one or more nucleic acid sequences encoding the one or more therapeutic proteins are operatively linked to a promoter. In some embodiments, the expression cassette includes one or more of an enhancer and/or promoter, a 3′ untranslated region (with or without introns), a translation initiation region, a transgene (protein coding regions, which could themselves be broken down into several components), a 5′ untranslated region, and a polyadenylation addition region.

In some embodiments, the non-viral DNA vectors are substantially devoid of CpG sequences. In some embodiments, the non-viral DNA vectors comprise less than about 500 CpG per vector, less than about 450 CpG per vector, less than about 400 CpG per vector, less than about 350 CpG per vector, less than about 300 CpG per vector, less than about 250 CpG per vector, less than about 200 CpG per vector, less than about 150 CpG per vector, less than about 100 CpG per vector, less than about 75 CpG per vector, less than about 50 CpG per vector, less than about 25 CpG per vector, less than about 20 CpG per vector, less than about 15 CpG per vector, less than about 10 CpG per vector, etc.

In further embodiments, the circular, non-viral DNA vector comprises (i) a first portion comprising an expression cassette including one or more nucleic acid sequences encoding one or more therapeutic proteins, where each of the one or more nucleic acid sequences encoding the one or more therapeutic proteins are operatively linked to a promoter; and (ii) a second portion capable of forming at least one cruciform structure, wherein the second portion comprises at least two inverted repeat sequences, wherein the at least two inverted repeat sequences are separated by at least a portion of a bacterial origin of replication. In some embodiments, each of the inverted repeat sequences comprise or are derived from nucleic acid sequences present within AAV serotypes. In some embodiments, the cruciform structure comprises a Holliday junction.

In some embodiments, the at least the portion of the bacterial origin of replication is single stranded.

In some embodiments, the non-viral DNA vectors are substantially devoid of CpG sequences. In some embodiments, the non-viral DNA vectors comprise less than about 500 CpG per vector, less than about 450 CpG per vector, less than about 400 CpG per vector, less than about 350 CpG per vector, less than about 300 CpG per vector, less than about 250 CpG per vector, less than about 200 CpG per vector, less than about 150 CpG per vector, less than about 100 CpG per vector, less than about 75 CpG per vector, less than about 50 CpG per vector, less than about 25 CpG per vector, less than about 20 CpG per vector, less than about 15 CpG per vector, less than about 10 CpG per vector, etc.

In yet further embodiments, the circular, non-viral DNA vector comprises: (i) a first portion comprising an expression cassette including one or more nucleic acid sequences encoding one or more therapeutic proteins, where each of the one or more nucleic acid sequences encoding the one or more therapeutic proteins are operatively linked to a promoter; and (ii) a second portion capable of forming at least one cruciform structure, wherein the second portion has the Formula X—Y—X′, where X and X′ are each inverted repeat sequences, and where Y comprises at least a portion of a bacterial origin of replication. In some embodiments, the inverted repeat sequences comprise or are derived from nucleic acid sequences present within AAV genomes. In some embodiments, the at least the portion of the bacterial origin of replication is single stranded when the repeat sequences form a cruciform structure. In some embodiments, the cruciform structure comprises a Holliday junction.

In some embodiments, the non-viral DNA vectors are substantially devoid of CpG sequences. In some embodiments, the non-viral DNA vectors comprise less than about 500 CpG per vector, less than about 450 CpG per vector, less than about 400 CpG per vector, less than about 350 CpG per vector, less than about 300 CpG per vector, less than about 250 CpG per vector, less than about 200 CpG per vector, less than about 150 CpG per vector, less than about 100 CpG per vector, less than about 75 CpG per vector, less than about 50 CpG per vector, less than about 25 CpG per vector, less than about 20 CpG per vector, less than about 15 CpG per vector, less than about 10 CpG per vector, etc.

In even further embodiments, the non-viral DNA vector comprises the following elements operatively linked in a 5′ to a 3′ direction: (i) a first repeat sequence; (ii) a minimal bacterial origin of replication; (iii) a second repeat sequence; and (iv) an expression cassette. In some embodiments, the first and second repeat sequences comprise or are derived from nucleic acid sequences present within AAV genomes. In some embodiments, at least a portion of the minimal bacterial origin of replication is single stranded when the repeat sequences for a cruciform structure. In some embodiments, the cruciform structure comprises a Holliday junction.

In some embodiments, the expression cassette includes one or more nucleic acid sequences encoding one or more therapeutic proteins, where each of the one or more nucleic acid sequences encoding the one or more therapeutic proteins are operatively linked to a promoter. In some embodiments, the expression cassette includes one or more of an enhancer and/or promoter, a 3′ untranslated region (with or without introns), a translation initiation region, a transgene (protein coding regions, which could themselves be broken down into several components), a 5′ untranslated region, and a polyadenylation addition region.

In some embodiments, the non-viral DNA vectors are substantially devoid of CpG sequences. In some embodiments, the non-viral DNA vectors comprise less than about 500 CpG per vector, less than about 450 CpG per vector, less than about 400 CpG per vector, less than about 350 CpG per vector, less than about 300 CpG per vector, less than about 250 CpG per vector, less than about 200 CpG per vector, less than about 150 CpG per vector, less than about 100 CpG per vector, less than about 75 CpG per vector, less than about 50 CpG per vector, less than about 25 CpG per vector, less than about 20 CpG per vector, less than about 15 CpG per vector, less than about 10 CpG per vector, etc.

In yet even further embodiments, the circular, non-viral DNA vector comprises (i) a first portion comprising an expression cassette including one or more nucleic acid sequences encoding one or more therapeutic proteins, where each of the one or more nucleic acid sequences encoding the one or more therapeutic proteins are operatively linked to a promoter; and (ii) a second portion capable of forming at least one cruciform structure, wherein the second portion comprises at least two inverted repeat sequences separated by a non-repeating nucleotide sequence comprising a heterologous gene or a portion of a heterologous gene. In some embodiments, a least a portion of a bacterial origin of replication is included within the circular non, viral DNA vector, but not within the second portion. In some embodiments, at least a portion of a bacterial origin of replication is included in the first portion or within a third portion adjacent to either the first or second portions, but not within the second portion. In some embodiments, the cruciform structure comprises a Holliday junction. In some embodiments, the inverted repeat sequences comprise or are derived from nucleic acid sequences present within AAV serotypes. In some embodiments, the non-repeating nucleotide sequence comprising the heterologous gene, or the portion thereof is single stranded, while the remainder of the second portion is double stranded.

In some embodiments, the non-viral DNA vectors are substantially devoid of CpG sequences. In some embodiments, the non-viral DNA vectors comprise less than about 500 CpG per vector, less than about 450 CpG per vector, less than about 400 CpG per vector, less than about 350 CpG per vector, less than about 300 CpG per vector, less than about 250 CpG per vector, less than about 200 CpG per vector, less than about 150 CpG per vector, less than about 100 CpG per vector, less than about 75 CpG per vector, less than about 50 CpG per vector, less than about 25 CpG per vector, less than about 20 CpG per vector, less than about 15 CpG per vector, less than about 10 CpG per vector, etc.

In some embodiments, each of inverted repeat sequences are nucleic acid sequences which include between about 20 to about 500 bp, from between about 20 bp and about 450 bp, from between about 20 bp to about 400 bp, from between about 20 bp to about 300 bp, from between about 20 bp to about 250 bp, from between about 20 bp to about 200 bp, from between about 20 bp to about 160 bp, from between about 60 bp to about 160 bp, from about 70 bp to about 150 bp, from about 80 bp to about 140 bp, from about 90 bp to about 130 bp, etc. In some embodiments, each of the inverted repeat sequences have about 115 bp, 116 bp, 117 bp, 118 bp, 119 bp, 120 bp, 121 bp, 122 bp, 123 bp, 124 bp, 125 bp, 126 bp, 127 bp, 128 bp, 129 bp, 130 bp, 131 bp, 132 bp, 133 bp, 134 bp, 135 bp, 136 bp, 137 bp, 138 bp, 139 bp, 140 bp, 141 bp, 142 bp, etc.

In some embodiments, each of the inverted repeat sequences have a nucleic acid sequence having at least 80% identity to any one of SEQ IDS NO: 1-18. In some embodiments, each of the inverted repeat sequences have a nucleic acid sequence having at least 85% identity to any one of SEQ IDS NO: 1-18. In some embodiments, each of the inverted repeat sequences have a nucleic acid sequence having at least 90% identity to any one of SEQ IDS NO: 1-18.

In some embodiments, each of the inverted repeat sequences have a nucleic acid sequence having at least 91% identity to any one of SEQ IDS NO: 1-18. In some embodiments, each of the inverted repeat sequences have a nucleic acid sequence having at least 92% identity to any one of SEQ IDS NO: 1-18. In some embodiments, each of the inverted repeat sequences have a nucleic acid sequence having at least 93% identity to any one of SEQ IDS NO: 1-18. In some embodiments, each of the inverted repeat sequences have a nucleic acid sequence having at least 94% identity to any one of SEQ IDS NO: 1-18. In some embodiments, each of the inverted repeat sequences have a nucleic acid sequence having at least 95% identity to any one of SEQ IDS NO: 1-18. In some embodiments, each of the inverted repeat sequences have a nucleic acid sequence having at least 96% identity to any one of SEQ IDS NO: 1-18. In some embodiments, each of the inverted repeat sequences have a nucleic acid sequence having at least 97% identity to any one of SEQ IDS NO: 1-18. In some embodiments, each of the inverted repeat sequences have a nucleic acid sequence having at least 98% identity to any one of SEQ IDS NO: 1-18. In some embodiments, each of the inverted repeat sequences have a nucleic acid sequence having at least 99% identity to any one of SEQ IDS NO: 1-18. In some embodiments, each of the inverted repeat sequences have a nucleic acid sequence having any one of SEQ IDS NO: 1-18.

In some embodiments, each of the inverted repeat sequences comprise or are derived from AAV inverted terminal repeat elements (e.g., derived from elements of an AAV serotype, such as any one of AAV1, AAV2, AAV3, AAV4, AAV5, AAV6, and/or AAV7). For instance, each of the inverted repeat groups may include one or more of the A, A′, B, B′, C, C′, D, and/or D′ elements of inverted terminal repeat elements from one or more AAV serotypes.

In some embodiments, a 5′ A element comprises at least 90% identity to TTGGCCACTCCCTCTCTGCGCGCTDGCTCGCTCACTGAGGC (SEQ ID NO: 74). In some embodiments, a 5′ A element comprises TTGGCCACTCCCTCTCTGCGCGCTDGCTCGCTCACTGAGGC (SEQ ID NO: 74). In some embodiments, a 3′ A element comprises at least 90% identity to GCCTCAGTGAGCGAGCGAGCGCGCAGAGAGGGAGTGGCCAA (SEQ ID NO: 75). In some embodiments, a 3′ A element comprises GCCTCAGTGAGCGAGCGAGCGCGCAGAGAGGGAGTGGCCAA (SEQ ID NO: 75). In some embodiments, a 5′ B element comprises at least 90% identity to CGGGCGACC (SEQ ID NO: 76). In some embodiments, a 5′ B element comprises CGGGCGACC (SEQ ID NO: 76). In some embodiments, a 3′ B element comprises at least 90% identity to GGTCGCCCG (SEQ ID NO: 77). In some embodiments, a 3′ B element comprises GGTCGCCCG (SEQ ID NO: 77. In some embodiments, a 5′ C element comprises at least 90% identity to CGCCCGGGC (SEQ ID NO: 78). In some embodiments, a 5′ C element comprises CGCCCGGGC (SEQ ID NO: 78). In some embodiments, a 3′ C element comprises at least 90% identity to GCCCGGGCG (SEQ ID NO: 79). In some embodiments, a 3′ C element comprises GCCCGGGCG (SEQ ID NO: 79). In some embodiments, a 5′ D element comprises at least 90% identity to AGGAACCCCTAGTGATGGAG (SEQ ID NO: 80). In some embodiments, a 5′ D element comprises identity to AGGAACCCCTAGTGATGGAG (SEQ ID NO: 80). In some embodiments, a 3′ D element comprises at least 90% identity to CTCCATCACTAGGGGTTCCT (SEQ ID NO: 81). In some embodiments, a 3′ D element comprises identity to CTCCATCACTAGGGGTTCCT (SEQ ID NO: 81).

In some embodiments, a 5′ A element comprises at least 95% identity to TTGGCCACTCCCTCTCTGCGCGCTDGCTCGCTCACTGAGGC (SEQ ID NO: 74). In some embodiments, a 5′ A element comprises TTGGCCACTCCCTCTCTGCGCGCTDGCTCGCTCACTGAGGC (SEQ 1D NO: 74). In some embodiments, a 3′ A element comprises at least 95% identity to GCCTCAGTGAGCGAGCGAGCGCGCAGAGAGGGAGTGGCCAA (SEQ ID NO: 75). In some embodiments, a 3′ A element comprises GCCTCAGTGAGCGAGCGAGCGCGCAGAGAGGGAGTGGCCAA (SEQ ID NO: 75). In some embodiments, a 5′ B element comprises at least 95% identity to CGGGCGACC (SEQ ID NO: 76). In some embodiments, a 5′ B element comprises CGGGCGACC (SEQ ID NO: 76). In some embodiments, a 3′ B element comprises at least 95% identity to GGTCGCCCG (SEQ ID NO: 77). In some embodiments, a 3′ B element comprises GGTCGCCCG (SEQ ID NO: 77. In some embodiments, a 5′ C element comprises at least 95% identity to CGCCCGGGC (SEQ ID NO: 78). In some embodiments, a 5′ C element comprises CGCCCGGGC (SEQ ID NO: 78). In some embodiments, a 3′ C element comprises at least 95% identity to GCCCGGGCG (SEQ ID NO: 79). In some embodiments, a 3′ C element comprises GCCCGGGCG (SEQ ID NO: 79). In some embodiments, a 5′ D element comprises at least 95% identity to AGGAACCCCTAGTGATGGAG (SEQ ID NO: 80). In some embodiments, a 5′ D element comprises identity to AGGAACCCCTAGTGATGGAG (SEQ ID NO: 80). In some embodiments, a 3′ D element comprises at least 95% identity to CTCCATCACTAGGGGTTCCT (SEQ ID NO: 81). In some embodiments, a 3′ D element comprises identity to CTCCATCACTAGGGGTTCCT (SEQ ID NO: 81).

In some embodiments, a 5′ A element comprises at least 97% identity to TTGGCCACTCCCTCTCTGCGCGCTDGCTCGCTCACTGAGGC (SEQ ID NO: 74). In some embodiments, a 5′ A element comprises TTGGCCACTCCCTCTCTGCGCGCTDGCTCGCTCACTGAGGC (SEQ ID NO: 74). In some embodiments, a 3′ A element comprises at least 97% identity to GCCTCAGTGAGCGAGCGAGCGCGCAGAGAGGGAGTGGCCAA (SEQ ID NO: 75). In some embodiments, a 3′ A element comprises GCCTCAGTGAGCGAGCGAGCGCGCAGAGAGGGAGTGGCCAA (SEQ ID NO: 75). In some embodiments, a 5′ B element comprises at least 97% identity to CGGGCGACC (SEQ ID NO: 76). In some embodiments, a 5′ B element comprises CGGGCGACC (SEQ ID NO: 76). In some embodiments, a 3′ B element comprises at least 97% identity to GGTCGCCCG (SEQ ID NO: 77). In some embodiments, a 3′ B element comprises GGTCGCCCG (SEQ ID NO: 77. In some embodiments, a 5′ C element comprises at least 97% identity to CGCCCGGGC (SEQ lD NO: 78). In some embodiments, a 5′ C element comprises CGCCCGGGC (SEQ ID NO: 78). In some embodiments, a 3′ C element comprises at least 97% identity to GCCCGGGCG (SEQ ID NO: 79). In some embodiments, a 3′ C element comprises GCCCGGGCG (SEQ ID NO: 79). In some embodiments, a 5′ D element comprises at least 97% identity to AGGAACCCCTAGTGATGGAG (SEQ ID NO: 80). In some embodiments, a 5′ D element comprises identity to AGGAACCCCTAGTGATGGAG (SEQ ID NO: 80). In some embodiments, a 3′ D element comprises at least 97% identity to CTCCATCACTAGGGGTTCCT (SEQ ID NO: 81). In some embodiments, a 3′ D element comprises identity to CTCCATCACTAGGGGTTCCT (SEQ ID NO: 81).

In some embodiments, a 5′ A element comprises at least 99% identity to TTGGCCACTCCCTCTCTGCGCGCTDGCTCGCTCACTGAGGC (SEQ ID NO: 74). In some embodiments, a 5′ A element comprises TTGGCCACTCCCTCTCTGCGCGCTDGCTCGCTCACTGAGGC (SEQ ID NO: 74). In some embodiments, a 1′ A element comprises at least 99% identity to GCCTCAGTGAGCGAGCGAGCGCGCAGAGAGGGAGTGGCCAA (SEQ ID NO: 75). In some embodiments, a 3′ A element comprises GCCTCAGTGAGCGAGCGAGCGCGCAGAGAGGGAGTGGCCAA (SEQ ID NO: 75). In some embodiments, a 5′ B element comprises at least 99% identity to CGGGCGACC (SEQ ID NO: 76). In some embodiments, a 5′ B element comprises CGGGCGACC (SEQ ID NO: 76). In some embodiments, a 3′ B element comprises at least 99% identity to GGTCGCCCG (SEQ ID NO: 77). In some embodiments, a 3′ B element comprises GGTCGCCCG (SEQ ID NO: 77. In some embodiments, a 5′ C element comprises at least 99% identity to CGCCCGGGC (SEQ ID NO: 78). In some embodiments, a 5′ C element comprises CGCCCGGGC (SEQ ID NO: 78). In some embodiments, a 3′ C element comprises at least 99% identity to GCCCGGGCG (SEQ ID NO: 79). In some embodiments, a 3′ C element comprises GCCCGGGCG (SEQ ID NO: 79). In some embodiments, a 5′ D element comprises at least 99% identity to AGGAACCCCTAGTGATGGAG (SEQ ID NO: 80). In some embodiments, a 5′ D element comprises identity to AGGAACCCCTAGTGATGGAG (SEQ ID NO: 80). In some embodiments, a 3′ D element comprises at least 99% identity to CTCCATCACTAGGGGTTCCT (SEQ ID NO: 81). In some embodiments, a 3′ D element comprises identity to CTCCATCACTAGGGGTTCCT (SEQ ID NO: 81).

In some embodiments, a first inverted repeat sequence may have the Formula D-A-C-C′-B-B′-A′, whereas a second inverted repeat sequence may have the Formula -A-B-B′-C-C′-A′-D′. In some embodiments, a first inverted repeat sequence may have the Formula D-A-C-C′-B-B′-A′, whereas a second inverted repeat sequence may have the Formula -A-B-B′-C-C′-A′-D′, but do not include a DD element as described herein. As noted herein, each of these first and second inverted repeat sequences are separated by a non-repeating nucleotide sequence having at least 3 nucleotides (e.g., at least a portion of a bacterial origin of replication, such as at least a portion of a bacterial origin of replication derived from R6K0).

In some embodiments, the non-viral DNA vectors of the present disclosure do not include or form a DD-ITR, “double D” ITR, or “DD element.” In some embodiments, the non-viral DNA vectors do not comprise a DD element but do include at least a portion of a bacterial origin of replication. In some embodiments, the circular, non-viral DNA vectors lack a drug resistance gene and do not include a DD element. Said another way, the D and D′ elements included within the inverted repeat sequences of the presently disclosed non-viral DNA vector are separated by a heterologous, non-repeat element which may contain at least a portion of a bacterial origin of replication. Surprisingly, Applicant has discovered that the presently disclosed non-viral vectors which are devoid of a “DD element” direct higher amounts of linked heterologous gene expression and may persist in vivo as long, or longer, as those vectors which do include a “DD element.” Furthermore, the structure of the repeat elements within the non-viral DNA vector of the present disclosure are not formed through the process of circularization of a viral genome (e.g., an AAV genome, or AAV vector genome) or linear DNA fragment via the inverted terminal repeat sequences.

In some embodiments, the inverted repeat sequences are contiguous with and flank the non-repeated nucleic acid sequence having at least three nucleotides. In some embodiments, the non-repeating nucleic acid sequence having the at least three nucleotides comprises at least a portion of a bacterial origin of replication. In some embodiments, the at least the portion of the bacterial origin of replication is derived from plasmid R6K (e.g., its y origin of replication (oriR6Kγ). In some embodiments, the R6K-derived bacterial origin of replication has been modified to reduce CpG content, e.g., to reduce CpG content by at least 60%, at least 50%, at least 40%, at least 30%, at least 25%, at least 20%, at least 10%, at least 5%, etc. In some embodiments, the at least the portion of the bacterial origin of replication includes one or more internal direct repeat sequences. In some embodiments, the at least the portion of the bacterial origin of replication includes one or more regions of internal secondary structure which, it are believed to be important in helping to maintain the cruciform structure.

In some embodiments, the at least the portion of the bacterial origin of replication has at least 90% identity to any of one SEQ ID NOS: 58-59. In some embodiments, the at least the portion of the bacterial origin of replication has at least 91% identity to any of one SEQ ID NOS: 58-59. In some embodiments, the at least the portion of the bacterial origin of replication has at least 92% identity to any of one SEQ ID NOS: 58-59. In some embodiments, the at least the portion of the bacterial origin of replication has at least 93% identity to any of one SEQ ID NOS: 58-59. In some embodiments, the at least the portion of the bacterial origin of replication has at least 94% identity to any of one SEQ ID NOS: 58-59. In some embodiments, the at least the portion of the bacterial origin of replication has at least 95% identity to any of one SEQ ID NOS: 58-59. In some embodiments, the at least the portion of the bacterial origin of replication has at least 96% identity to any of one SEQ ID NOS: 58-59. In some embodiments, the at least the portion of the bacterial origin of replication has at least 97% identity to any of one SEQ ID NOS: 58-59. In some embodiments, the at least the portion of the bacterial origin of replication has at least 98% identity to any of one SEQ ID NOS: 58-59. In some embodiments, the at least the portion of the bacterial origin of replication has at least 99% identity to any of one SEQ ID NOS: 58-59. In some embodiments, the at least the portion of the bacterial origin of replication has any of one SEQ ID NOS: 58-59.

In some embodiments, the second portion comprises a first nucleic acid sequence having at least 90% identity to any one of SEQ ID NOS: 1, 3, 5, 7, 9, 11, 13, 15, or 17; a second nucleic acid sequence having at least 90% identity to any one of SEQ ID NOS: 58-59; and a third nucleic acid sequence having at least 90% identity to any one of SEQ ID NOS: 2, 4, 6, 8, 10, 12, 14, 16, or 18. In some embodiments, the second portion comprises a first nucleic acid sequence having at least 91% identity to any one of SEQ ID NOS: 1, 3, 5, 7, 9, 11, 13, 15, or 17; a second nucleic acid sequence having at least 91% identity to any one of SEQ ID NOS: 58-59; and a third nucleic acid sequence having at least 91% identity to any one of SEQ ID NOS: 2, 4, 6, 8, 10, 12, 14, 16, or 18. In some embodiments, the second portion comprises a first nucleic acid sequence having at least 92% identity to any one of SEQ ID NOS: 1, 3, 5, 7, 9, 11, 13, 15, or 17; a second nucleic acid sequence having at least 92% identity to any one of SEQ ID NOS: 58-59; and a third nucleic acid sequence having at least 92% identity to any one of SEQ ID NOS: 2, 4, 6, 8, 10, 12, 14, 16, or 18. In some embodiments, the second portion comprises a first nucleic acid sequence having at least 93% identity to any one of SEQ ID NOS: 1, 3, 5, 7, 9, 11, 13, 15, or 17; a second nucleic acid sequence having at least 93% identity to any one of SEQ ID NOS: 58-59; and a third nucleic acid sequence having at least 93% identity to any one of SEQ ID NOS: 2, 4, 6, 8, 10, 12, 14, 16, or 18. In some embodiments, the second portion comprises a first nucleic acid sequence having at least 94% identity to any one of SEQ ID NOS: 1, 3, 5, 7, 9, 11, 13, 15, or 17; a second nucleic acid sequence having at least 94% identity to any one of SEQ ID NOS: 58-59; and a third nucleic acid sequence having at least 94% identity to any one of SEQ ID NOS: 2, 4, 6, 8, 10, 12, 14, 16, or 18.

In some embodiments, the second portion comprises a first nucleic acid sequence having at least 95% identity to any one of SEQ ID NOS: 1, 3, 5, 7, 9, 11, 13, 15, or 17; a second nucleic acid sequence having at least 95% identity to any one of SEQ ID NOS: 58-59; and a third nucleic acid sequence having at least 95% identity to any one of SEQ ID NOS: 2, 4, 6, 8, 10, 12, 14, 16, or 18. In some embodiments, the second portion comprises a first nucleic acid sequence having at least 96% identity to any one of SEQ ID NOS: 1, 3, 5, 7, 9, 11, 13, 15, or 17; a second nucleic acid sequence having at least 96% identity to any one of SEQ ID NOS: 58-59; and a third nucleic acid sequence having at least 96% identity to any one of SEQ ID NOS: 2, 4, 6, 8, 10, 12, 14, 16, or 18. In some embodiments, the second portion comprises a first nucleic acid sequence having at least 97% identity to any one of SEQ ID NOS: 1, 3, 5, 7, 9, 11, 13, 15, or 17; a second nucleic acid sequence having at least 97% identity to any one of SEQ ID NOS: 58-59; and a third nucleic acid sequence having at least 97% identity to any one of SEQ ID NOS: 2, 4, 6, 8, 10, 12, 14, 16, or 18. In some embodiments, the second portion comprises a first nucleic acid sequence having at least 98% identity to any one of SEQ ID NOS: 1, 3, 5, 7, 9, 11, 13, 15, or 17; a second nucleic acid sequence having at least 98% identity to any one of SEQ ID NOS: 58-59; and a third nucleic acid sequence having at least 98% identity to any one of SEQ ID NOS: 2, 4, 6, 8, 10, 12, 14, 16, or 18. In some embodiments, the second portion comprises a first nucleic acid sequence having at least 99% identity to any one of SEQ ID NOS: 1, 3, 5, 7, 9, 11, 13, 15, or 17; a second nucleic acid sequence having at least 99% identity to any one of SEQ ID NOS: 58-59; and a third nucleic acid sequence having at least 99% identity to any one of SEQ ID NOS: 2, 4, 6, 8, 10, 12, 14, 16, or 18. In some embodiments, the second portion comprises a first nucleic acid sequence having any one of SEQ ID NOS: 1, 3, 5, 7, 9, 11, 13, 15, or 17; a second nucleic acid sequence having any one of SEQ ID NOS: 58-59; and a third nucleic acid sequence having any one of SEQ ID NOS: 2, 4, 6, 8, 10, 12, 14, 16, or 18.

In some embodiments, the second portion comprises a nucleic acid sequence having at least 90% identity to any one of SEQ ID NOS: 35 and 60. In some embodiments, the second portion comprises a nucleic acid sequence having at least 91% identity to any one of SEQ ID NOS: 35 and 60. In some embodiments, the second portion comprises a nucleic acid sequence having at least 92% identity to any one of SEQ ID NOS: 35 and 60. In some embodiments, the second portion comprises a nucleic acid sequence having at least 93% identity to any one of SEQ ID NOS: 35 and 60. In some embodiments, the second portion comprises a nucleic acid sequence having at least 94% identity to any one of SEQ ID NOS: 35 and 60. In some embodiments, the second portion comprises a nucleic acid sequence having at least 95% identity to any one of SEQ ID NOS: 35 and 60. In some embodiments, the second portion comprises a nucleic acid sequence having at least 96% identity to any one of SEQ ID NOS: 35 and 60. In some embodiments, the second portion comprises a nucleic acid sequence having at least 97% identity to any one of SEQ ID NOS: 35 and 60. In some embodiments, the second portion comprises a nucleic acid sequence having at least 98% identity to any one of SEQ ID NOS: 35 and 60. In some embodiments, the second portion comprises a nucleic acid sequence having at least 99% identity to any one of SEQ ID NOS: 35 and 60. In some embodiments, the second portion comprises a nucleic acid sequence having SEQ ID NO: 60.

In some embodiments, the circular, non-viral DNA vectors comprise a nucleic acid sequence having at least 85% identity to any one of SEQ ID NOS: 28-30, 38, 40-48, and 72-73. In some embodiments, the circular, non-viral DNA vectors comprise a nucleic acid sequence having at least 90% identity to any one of SEQ ID NOS: 28-30, 38, 40-48, and 72-73. In some embodiments, the circular, non-viral DNA vectors comprise a nucleic acid sequence having at least 91% identity to any one of SEQ ID NOS: 28-30, 38, 40-48, and 72-73. In some embodiments, the circular, non-viral DNA vectors comprise a nucleic acid sequence having at least 92% identity to any one of SEQ ID NOS: 28-30, 38, 40-48, and 72-73. In some embodiments, the circular, non-viral DNA vectors comprise a nucleic acid sequence having at least 93% identity to any one of SEQ ID NOS: 28-30, 38, 40-48, and 72-73. In some embodiments, the circular, non-viral DNA vectors comprise a nucleic acid sequence having at least 94% identity to any one of SEQ ID NOS: 28-30, 38, 40-48, and 72-73. In some embodiments, the circular, non-viral DNA vectors comprise a nucleic acid sequence having at least 95% identity to any one of SEQ ID NOS: 28-30, 38, 40-48, and 72-73. In some embodiments, the circular, non-viral DNA vectors comprise a nucleic acid sequence having at least 96% identity to any one of SEQ ID NOS: 28-30, 38, 40-48, and 72-73. In some embodiments, the circular, non-viral DNA vectors comprise a nucleic acid sequence having at least 97% identity to any one of SEQ ID NOS: 28-30, 38, 40-48, and 72-73. In some embodiments, the circular, non-viral DNA vectors comprise a nucleic acid sequence having at least 98% identity to any one of SEQ ID NOS: 28-30, 38, 40-48, and 72-73. In some embodiments, the circular, non-viral DNA vectors comprise a nucleic acid sequence having at least 99% identity to any one of SEQ ID NOS: 28-30, 38, 40-48, and 72-73. In some embodiments, the circular, non-viral DNA vectors comprise a nucleic acid sequence having any one of SEQ ID NOS: 28-30, 38, 40-48, and 72-73.

### One or More Nucleic Acid Sequences Encoding One or More Peptides or Polypeptides

In some embodiments, the circular, non-viral DNA vectors of the present disclosure include one or more nucleic acid sequences encoding one or more peptides, polypeptides, etc. In some embodiments, the circular, non-viral DNA vectors of the present disclosure include one or more nucleic acid sequences encoding one or more reporter genes. In yet other embodiments, the circular, non-viral DNA vectors of the present disclosure include one or more heterologous genes encoding one or more therapeutic proteins.

In some embodiments, the one or more nucleic acid sequences encoding the one or more therapeutic proteins ranges in size from between about 1 Kb to about 150 Kb, e.g., from between about 1 Kb to about 120 Kb, from between about 1 Kb to about 100 Kb, from between about 1 Kb to about 80 Kb, from between about 1 Kb to about 60 Kb, from between about 1 Kb to about 40 Kb, from between about 1 Kb to about 30 Kb, from between about 1 Kb to about 25 Kb, from between about 1 Kb to about 20 Kb, from between about 1 Kb to about 15 Kb, from between about 1 Kb to about 12 Kb, from between about 1 Kb to about 11 Kb, from between about 1 Kb to about 10 Kb, from between about 1 Kb to about 9 Kb, from between about 1 Kb to about 8 Kb, from between about 1 Kb to about 7 Kb, from between about 1 Kb to about 6 Kb, etc. In some embodiments, the one or more nucleic acid sequences encoding the one or more therapeutic proteins has a size of about 5 Kb, about 6 Kb, about 7 Kb, about 7.5 Kb, about 8 Kb, about 8.5 Kb, about 9 Kb, about 9.5 Kb, about 10 Kb, about 10.5 Kb, about 11 Kb, about 12 Kb, about 13 Kb, about 14 Kb, about 15 Kb, about 16 Kb, about 17 Kb, about 18 Kb, about 19 Kb, about 20 Kb, about 21 Kb, etc.

Therapeutic proteins include, but are not limited to, ALPL, PCSK9, PCSK7, SerpinA1, ABCB4 (having at least 80%, at least 85%, at least 90%, at least 95%, at least 97%, at least 99% identity to any one of SEQ ID NOS: 64, 65, 66, or 69), ATP7B (having at least 80%, at least 85%, at least 90%, at least 95%, at least 97%, at least 99% identity to any one of SEQ ID NOS: 69, 70, or 83), A1AT (having at least 80%, at least 85%, at least 90%, at least 95%, at least 97%, at least 99% identity to any one of SEQ ID NOS: 66), ABCB11, antiCD19-antiCD3 (having at least 80%, at least 85%, at least 90%, at least 95%, at least 97%, at least 99% identity to any one of SEQ ID NOS: 67), BDFS (having at least 80%, at least 85%, at least 90%, at least 95%, at least 97%, at least 99% identity to any one of SEQ ID NOS: 68) and variants thereof. Examples of diseases which may be treated according to the methods of the claimed invention include, but are not limited to, hypophosphatasia, anti-alpha-1 antitrypsin deficiency disease (treated such as with a protein having at least 80%, at least 85%, at least 90%, at least 95%, at least 97%, at least 99% identity to that of SEQ ID NO: 82), familiar hypercholesterolemia, hyperlipidemia, and progressive familial intrahepatic cholestasis types 1, 2, and 3.

In some embodiments, the non-viral DNA vectors of the present disclosure include one or more nucleic acid sequences encoding an alkaline phosphatase or a variant thereof. In other embodiments, the non-viral DNA vectors of the present disclosure include a nucleic acid sequence encoding a polypeptide including amino acid sequence encoding an alkaline phosphatase.

For example, in some embodiments the polypeptide including an amino acid sequence encoding an alkaline phosphatase has any one of the Formulas (IA) to (IE):

[A]v-[B]-[C]w-[R]q-([D]x-[E]y)z (IA),

[A]-[B]-[C]-[R]q-([D]x-[E]y) (IB),

([A]-[B])-([D]x-[E]y)z (IC),

([A]-[B])-([E]y) (ID), and

[A]-[B]-[R]q-([E]y) (IE)
wherein
A comprises an amino acid sequence encoding a secretion signal peptide;
B comprises an amino acid encoding an alkaline phosphatase;
C comprises an amino acid sequence encoding a GPI anchor;
R is -(Mo(Fc)Np)-, where M and N each independently include between 1 and 6 amino acids, where Fc is a Fc domain, and o and p are each independently 0, 1, or 2;
D comprises an amino acid sequence having between 4 and 6 amino acids, or is F(G)tF, where each F is the same amino acid, G is an amino acid sequence having 3, 4, or 5 amino acids, and tis an integer ranging from 2-5;
E comprises an amino acid sequence having between 1 and 8 amino acids; q is 0 or 1;
v is 0 or 1; w is 0 or 1;
x is 0 or an integer ranging from 1 to 6;
y is 0 or an integer ranging from 1 to 16; and
z is 0 or an integer ranging from 1 to 6;
provided that when v is 1, w is 0, q is 1, o is 1, p is 1, N is the diamino acid -D-I-, M is the diamino acid -L-K-, [B] comprises SEQ ID NO: 11, Fc comprises SEQ ID NO: 130, and x is 0, then [E]y does not comprise ten to sixteen contiguous aspartic acid residues.

In some embodiments, the polypeptide of any one of the Formulas (IA) to (IE) is not conjugated to a dextran. In other embodiments, the polypeptide of any one of the Formulas (IA) to (IE) is catalytically competent to allow formation of hydroxyapatite crystals in bone.

In some embodiments, E comprises 3 amino acids. In some embodiments, the 3 amino acids are selected from aspartic acid, serine, lysine, threonine, tyrosine, alanine, methionine, valine, tryptophan, praline, arginine, glutamine. In other embodiments, the 3 amino acid is selected from aspartic acid and serine. In some embodiments, E is -D-S-S-. In other embodiments, E is -D-D-S-. In other embodiments, E is -D-D-D. In other embodiments, E is -D-S-S-.

In some embodiments, E comprises 3 amino acids, and y ranges from 1 to 16. In some embodiments, E comprises 3 amino acids, and y ranges from 1 to 12. In some embodiments, E comprises 3 amino acids, and y ranges from 2 to 12. In some embodiments, E comprises 3 amino acids, and y ranges from 1 to 10. In some embodiments, E comprises 3 amino acids, and y ranges from 2 to 10. In some embodiments, E comprises 3 amino acids, and y ranges from 1 to 8. In some embodiments, E comprises 3 amino acids, and y ranges from 2 to 8. In some embodiments, E comprises 3 amino acids, and y ranges from 1 to 6. In some embodiments, E comprises 3 amino acids, and y ranges from 3 to 6. In some embodiments, E comprises 3 amino acids, and y ranges from 3 to 6 and q is 0.

In some embodiments, E is -D-S-S-, and y ranges from 1 to 16. In some embodiments, E is -D-S-S-, and y ranges from 1 to 12. In some embodiments, E is -D-S-S-, and y ranges from 1 to 10. In some embodiments, E is aspartic acid, and y ranges from 1 to 8. In some embodiments, E is -D-S-S-, and y is 6, i.e. [E]y is [-DSS-]6. In other embodiments, E is -D-S-S-, y is 6 and z is 1. In some embodiments, E is -D-S-S-, y is 6 and q is 0. In other embodiments, E is -D-S-S-, y is 6, z is 1, and q is 0. In other embodiments, E is -D-S-S-, y is 6, z is 1, q is 0, and x is 0. In other embodiments, E is -D-S-S-, y is 6, z is 1, q is 0, and x is 2.

In some embodiments, E is -D-S-S-, y is 6 and q is 1. In other embodiments, E is -D-S-S-, y is 6, z is 1, and q is 1. In other embodiments, E is -D-S-S-, y is 6, z is 1, x is 0, and q is 1. In other embodiments, E is -D-S-S-, y is 6, z is 1, x is 2, and q is 1.

Other examples of polypeptides encoding a soluble alkaline phosphatase are described m PCT Publication No. WO/2022/051555, the disclosure of which is hereby incorporated by reference herein in its entirety.

In some embodiments, the non-viral DNA vectors comprise a nucleic acid sequence encode an amino acid sequence having at least 90% identity to any one of SEQ ID NOS: 19-21 and 23-24. In some embodiments, the non-viral DNA vectors comprise a nucleic acid sequence encode an amino acid sequence having at least 95% identity to any one of SEQ ID NOS: 19-21 and 23-24. In some embodiments, the non-viral DNA vectors comprise a nucleic acid sequence encode an amino acid sequence having at least 96% identity to any one of SEQ ID NOS: 19-21 and 23-24. In some embodiments, the non-viral DNA vectors comprise a nucleic acid sequence encode an amino acid sequence having at least 97% identity to any one of SEQ ID NOS: 19-21 and 23-24. In some embodiments, the non-viral DNA vectors comprise a nucleic acid sequence encode an amino acid sequence having at least 98% identity to any one of SEQ ID NOS: 19-21 and 23-24. In some embodiments, the non-viral DNA vectors comprise a nucleic acid sequence encode an amino acid sequence having at least 99% identity to any one of SEQ ID NOS: 19-21 and 23-24. In some embodiments, the non-viral DNA vectors comprise a nucleic acid sequence encode an amino acid sequence having SEQ ID NOS: 20-21 and 23-24.

In some embodiments, the one or more heterologous genes are reporter genes. As used herein, a “reporter gene” is any gene whose expression can be measured. In some embodiments, a reporter gene can have a previously determined reference range of detectable expression. In some embodiments, a reporter gene can express a selectable or screenable marker. In some embodiments, selectable markers may also be used to select for organisms or cells that contain exogenous genetic material.

Reporter genes can encode enzymes such as beta-lactamase, beta-galactosidase, murine secreted embryonic alkaline phosphatase (MUSEAP), and luciferase (for beta-lactamase, see WO 96/30540 to Tsien, published Oct. 3, 1996). Reporter genes can also encode fluorescent proteins, such as green fluorescent protein (GFP) or mutants thereof as they are known in the art or are later developed (see, U.S. Pat. No. 5,625,048. to Tsien, issued Apr. 29, 1997; WO 96/23810 to Tsien, published Aug. 8, 1996; WO 97/28261 to Tsien, published Aug. 7, 1997; and PCT/US97/12410 to Tsien, filed Jul. 16, 1996). The products of reporter genes can be detected using methods known in the art, such as the use of chromogenic or fluorogenic substrates for enzymes. Chromogenic or fluorogenic readouts can be detected using, for example, optical methods such as absorbance or fluorescence

Examples of selectable markers include, but are not limited to, a neo gene, which codes for kanamycin resistance and can be selected for using kanamycin, GUS, green fluorescent protein (GFP), neomycin phosphotransferase II (nptII), luciferase (LUX), or an antibiotic resistance coding sequence. In some embodiments, screenable markers can be used to monitor expression. In some embodiments, exemplary screenable markers include, by way of example, a P-glucuronidase or uidA gene (GUS) which encodes an enzyme for which various chromogenic substrates are known; a P-lactamase gene, a gene which encodes an enzyme for which various chromogenic substrates are known (e.g., PADAC, a chromogenic cephalosporin); a luciferase gene; a tyrosinase gene, which encodes an enzyme capable of oxidizing tyrosine to DOPA and dopaquinone which in turn condenses to melanin; and a-galactosidase, which can turn a chromogenic a-galactose substrate.

### Promoters

Any promoter utilized in the art may be utilized to drive expression of one or nucleic acid sequences within the expression vectors described herein, e.g., to drive expression of the nucleic acid sequence encoding the polypeptide. Tn some embodiments, the promoter is one which is functional in mammalian cells. High-level constitutive promoters are preferred for use in the vectors according to the present disclosure. Examples of such promoters include, without limitation, the retroviral Rous sarcoma virus (RSN) LTR promoter (optionally with the RSV enhancer), the cytomegalovirus (CMV) promoter (optionally with the CMV enhancer) [see, e.g., Boshart et al, Cell, 41:521-530 (1985)], the SN40 promoter, the dihydrofolate reductase promoter, the beta-actin promoter, the beta-active promoter linked to the enhancer derived from the cytomegalovirus (CMN) immediate early (IE) promoter, the phosphoglycerol kinase (PGK) promoter, and the EF 1a promoter [Invitrogen]. Inducible promoters are regulated by exogenously supplied compounds, including, the zinc-inducible sheep metallothionine (MT) promoter, the dexamethasone (Dex)-inducible mouse mammary tumor virus (MMTV) promoter, the T7 polymerase promoter system [WO 98/10088]; the ecdysone insect promoter [No et al, Proc. Natl. Acad. Sci. USA, 93:3346-3351 (1996)], the tetracycline-repressible system [Gossen et al, Proc. Natl. Acad. Sci. USA, 89:5547-5551 (1992)], the tetracycline-inducible system [Gossen et al, Science, 268:1766-1769 (1995); see also Harvey et al, Curr. Opin. Chem. Biol, 2:512-518 (1998)], the RU486-inducible system [Wang et al, Nat. Biotech., 15:239-243 (1997) and Wang et al, Gene Ther., 4:432-441 (1997)] and the rapamycin-inducible system [Magari et al, J Clin. Invest., 100:2865-2872 (1997)]. Other types of inducible promoters which may be useful in the present disclosure are those which are regulated by a specific physiological state, e.g., temperature, acute phase, a particular differentiation state of the cell, or in replicating cells only.

Illustrative ubiquitous expression control sequences suitable for use in particular embodiments include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, a short elongation factor 1-alpha (EFla-short) promoter, a long elongation factor 1-alpha (EF1 α-long) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), B-kinesin ((3-KIN), the human ROSA 26 locus Orions et al., Nature Biotechnology 25, 1477-1482 (2007)), a Ubiquitin C promoter (UBC), a phosphoglycerate kinase-1 (PGK) promoter, a cytomegalovirus enhancer/chicken B-actin (CAG) promoter, a B-actin promoter and a myeloproliferative sarcoma virus enhancer, negative control region deleted, d1587rev primer-binding site substituted (MND) promoter (Challita et al., J Viral. 69(2):748-55 (1995)).

In some embodiments, the promoter may be selected from a Cytomegalovirus (CMV) minimal promoter and, more preferably, from human CMV (hCMV) such as the hCMV immediate early promoter derived minimal promoter as described in, e.g., Gossen and Bujard (Proc. Natl. Acad. Sci. USA, 1992, 89: 5547-5551). Modified promoters also may be utilized, including insertion and deletion mutation of native promoters and combinations or permutations thereof. One example of a modified promoter is the “minimal CMV promoter” as described by Gossen and Bujard (Proc. Natl. Acad. Sci. USA, 1992, 89: 5547-5551). In any case, any promoter can be tested readily for its effectiveness in the tetracycline-responsive expression system described herein by substitution for the minimal CMV promoter described herein.

In some embodiments, the promoter is an MND promoter. In some embodiments, the promoter is an EF1α promoter. In some embodiments, the promoter is a CD11b promoter. In some embodiments, the promoter is a EFS promoter. In some embodiments, the promoter is a Ubc promoter. In some embodiments, the promoter is a CD68LPp promoter.

In some embodiments, the promoter is a tissue-specific promoter, where the tissue-specific promoter is used to achieve cell type specific, lineage specific, or tissue-specific expression of a desired polynucleotide sequence (e.g., to express a particular nucleic acid encoding a polypeptide in only a subset of cell types or tissues or during specific stages of development). Illustrative examples of tissue specific promoters include, but are not limited to: an B29 promoter (B cell expression), a runt transcription factor (CBFa2) promoter (stem cell specific expression), an CD14 promoter (monocytic cell expression), an CD43 promoter (leukocyte and platelet expression), an CD45 promoter (hematopoietic cell expression), an CD68 promoter (macrophage expression), a CYP450 3A4 promoter (hepatocyte expression), an desmin promoter (muscle expression), an elastase 1 promoter (pancreatic acinar cell expression, an endoglin promoter (endothelial cell expression), a fibroblast specific protein 1 promoter (FSP1) promoter (fibroblast cell expression), a fibronectin promoter (fibroblast cell expression), a fms-related tyrosine kinase 1 (FLT1) promoter (endothelial cell expression), a glial fibrillary acidic protein (GFAP) promoter (astrocyte expression), an insulin promoter (pancreatic beta cell expression), an integrin, alpha 2b (ITGA2B) promoter (megakaryocytes), an intracellular adhesion molecule 2 (ICAM-2) promoter (endothelial cells), an interferon beta (IFN-P) promoter (hematopoietic cells), a keratin 5 promoter (keratinocyte expression), a myoglobin (MB) promoter (muscle expression), a myogenic differentiation 1 (MYOD1) promoter (muscle expression), a nephrin promoter (podocyte expression), a bone gamma-carboxyglutamate protein 2 (OG-2) promoter (osteoblast expression), an 3-oxoacid CoA transferase 2B (Oxct2B) promoter, (haploid-spermatid expression), a surfactant protein B (SP-B) promoter (lung expression), a synapsin promoter (neuron expression), a Wiskott-Aldrich syndrome protein (WASP) promoter (hematopoietic cell expression).

In some embodiments, the native promoter for the transgene is utilized. In some embodiments, the native promoter may be preferred when it is desired that expression of the gene should mimic the native expression. In some embodiments, the native promoter may be used when expression of the gene must be regulated temporally or developmentally, or in a tissue-specific manner, or in response to specific transcriptional stimuli. In some embodiments, other native expression control elements, such as enhancer elements, polyadenylation sites or Kozak consensus sequences may also be used to mimic the native expression. In some embodiments, the transgene product or other desirable product to be expressed is operably linked to a tissue-specific promoter. For instance, if expression in skeletal muscle is desired, a promoter active in muscle should be used. These include the promoters from genes encoding skeletal a-actin, myosin light chain 2A, dystrophin, muscle creatine kinase, as well as synthetic muscle promoters with activities higher than naturally occurring promoters [see Li et al., Nat. Biotech, 17:241-245 (1999)]. Examples of promoters that are tissue-specific are known for liver [albumin, Miyatake et al. J Viral, 71:5124-32 (1997); Human thyroxine binding globulin (TBG) promoter (see Yan et al, Gene. 2012 15; 506(2):289-94 (2012), the disclosure of which is hereby incorporated by reference herein in its entirety; hepatitis B virus core promoter, Sandig et al, Gene Ther., 3:1002-9 (1996); and alpha-fetoprotein (AFP), Arbuthnot et al, Hum. Gene Ther, 7:1503-14 (1996)], bone [osteocalcin, Stein et al, Mol. Biol. Rep., 24:185-96 (1997); and bone sialoprotein, Chen et al, J Bone Miner. Res., 11:654-64 (1996)], lymphocytes [CD2, Hansal et al., J Immunol, 161:1063-8 (1998); immunoglobulin heavy chain; T cell receptor a chain], neuronal [neuron-specific enolase (NSE) promoter, Andersen et al. Cell. Mol. Neurobiol, 13:503-15 (1993); neurofilament light-chain gene, Piccioli et al., 1991, Proc. Natl. Acad. Sci. USA, 88:5611-5 (1991); and the neuron-specific vgf gene, Piccioli et al, Neuron 15:373-84 (1995)]; among others.

Transcription may be increased by inserting an enhancer sequence into the non-viral DNA vectors of the present disclosure. Enhancers are typically cis-acting elements of DNA, usually about 10 to 300 bp in length, that act on a promoter to increase its transcription. Many enhancer sequences are now known from mammalian genes (globin, elastase, albumin, alpha-fetoprotein, and insulin) and from eukaryotic cell viruses. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the antigen-specific polynucleotide sequence but is preferably located at a site 5′ from the promoter.

In some embodiments, the vectors of the present disclosure include an insulator element, e.g., a cHS insulator.

### Inhibitory Sequences of Toll-Like Receptor 9

It has been well demonstrated a central role for Toll-like receptor 9 (TLR9), an immune sensor of DNA, in detecting non-viral DNA vectors and activating innate immune and CD8+ T cell responses. TLRs are a family of innate immune sensors preserved across mammalian species that are found on endosomal or plasma membranes of immune or other cells. TLR9 normally senses DNA from invasion of pathogenic DNA viruses and bacteria containing unmethylated cytosine-phosphate-guanine (CpG) motifs. After binding to TLR9, CpG-rich motifs of DNA lead to its dimerization and activates TLR9 signaling through MyD88, promoting induction of type I interferons and pro-inflammatory cytokines. Innate immune responses, such as interferon induction, trigger an antiviral state among cells, while inflammation recruits other immune cells to the site of infection and primes adaptive immune responses.

One solution blocking TLR9 activation is to include specific short DNA oligonucleotides that antagonize TLR9 activation into the non-viral DNA vectors of the present disclosure. In some embodiments, the vectors of the present disclosure comprise one copy of such sequence (SEQ ID NO: 39). In some embodiments, the vectors of the present disclosure comprise two or more copies of such sequence (SEQ ID NO: 39).

### In Vivo Resolution

The non-viral DNA vectors of the present disclosures are resolved in vivo. In some embodiments, the non-viral DNA vectors enter the cell through an endosomal mechanism (receptor-mediated endocytosis, pinocytosis, phagocytosis, etc.) or a non-endosomal mechanism (electroporation, ultrasonication-induced microbubble, membrane fusion through fusogenic complex etc.). Once inside an endosome, the non-viral DNA vector “escapes” the endosome and enters into the cytosol before the endosome fuses with a lysosome and the DNA is degraded. It is believed that the formulation of the non-viral DNA vector determines how the DNA escapes the endosome.

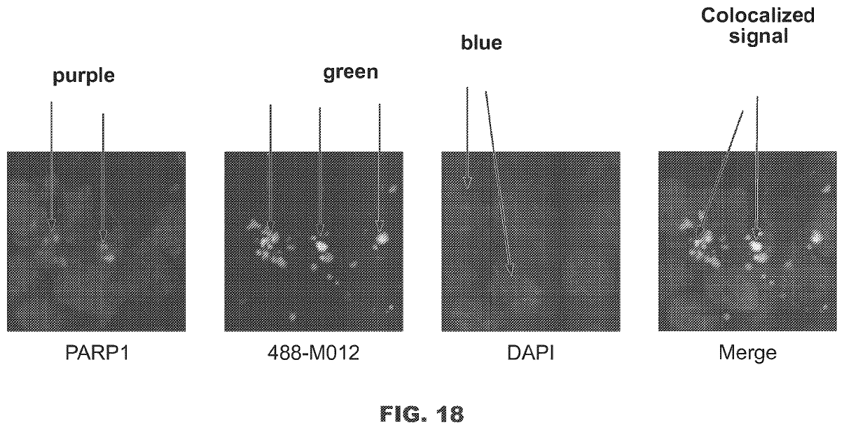

Once in the cytosol, the DNA needs to make its way into the nucleus in order to be expressed. It is believed that this process is assisted by the cruciform structure (repeat elements) of the presently disclosed circular, non-viral DNA vector. It is believed that cellular proteins, or protein complexes, known to bind this cruciform structure or known to be involved in Holliday junction resolution, have nuclear localization motifs. In other words, it believed that this structure is bound by proteins that direct its transport into the nucleus. As the proteins that help mediate nuclear translocation are the same (or a subset of) that mediate resolution, it's unclear if resolution happens in the cytoplasm or in the nucleus, although the expectation is that this occurs in the nucleus. For example, PARP1 was identified to co-localized with constructs according to the present disclosure in the cytoplasm of 293 cells post-transfection (see, e.g., FIG. 18). Generally speaking, PARP1 is a nuclear protein and might shuttle out to the cytoplasm to help DNA traffic into the nucleus.

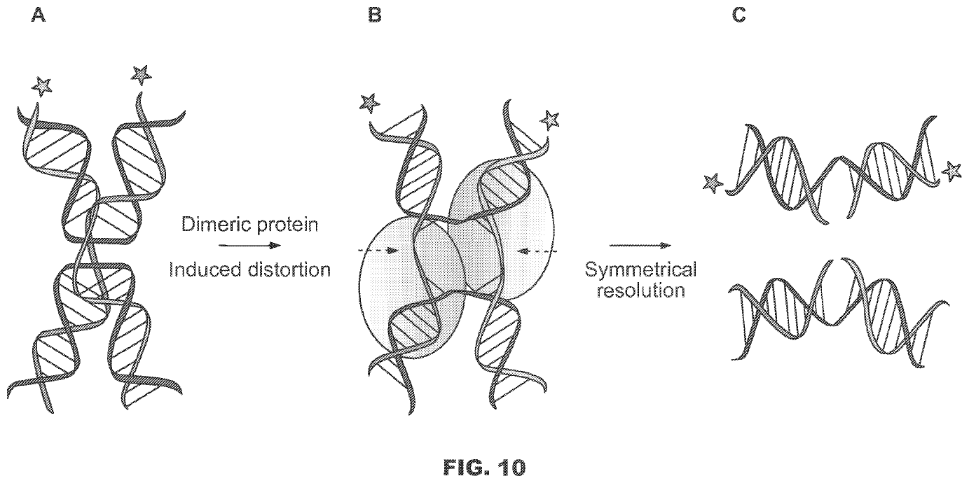

Although the process of “resolution” is largely unknown we can speak to the events that it would seem need to occur. Note that the process of normal endogenous Holliday junction resolution is also largely unknown and may occur through multiple pathways involving multiple different proteins/enzymes either in different cell types, or at different stages of the cell cycle. For instance, the first step is the recognition of the cruciform structure by proteins or protein complexes. This binding may be involved in nuclear localization as described above but is believed to be necessary to unwind the DNA a little to permit the next enzymatic steps. In some embodiments, the first enzymatic activity would cut the structure (see, e.g., FIGS. 10-11). After cutting, it is believed that a linear molecule is formed through a ligation process. It is believed that there are several DNA topoisomerases and/or DNA ligases that could been implicated in this process. It is also believed that there also several potential process/pathways via which this could occur. It is believed that DNA protein kinase (DNA-PK) is implicated in these pathways to convert the circular, non-viral vectors of the present disclosure into a linear form. In particular, DNA-PK is thought to be involved in the non-homologous end-joining (NHEJ) reaction and is believed to be part of a large multiprotein complex.

In some embodiments, other homology-mediated pathways may be involved in the formation of concatemers (see, e.g., FIG. 11).

### Pharmaceutical Compositions Comprising a Circular, Non-Viral DNA Vector

Another aspect of the present disclosure is directed to compositions comprising one or more circular, non-viral DNA vectors, e.g., circular, non-viral DNA vectors having at least 95%, 96%, 97%, 98%, or 99% identity to any one of SEQ ID NOS: 28-30, 38, 40-48 and 72-73.

In some embodiments, the present disclosure provides a composition comprising one or more of the circular, non-viral DNA vectors described herein and a carrier therefor (e.g., a pharmaceutically acceptable carrier). The composition desirably is a physiologically acceptable (e.g., pharmaceutically acceptable) composition, which comprises a carrier, e.g., a physiologically (e.g., pharmaceutically) acceptable carrier, and the non-viral DNA vector. Any suitable carrier can be used within the context of the present disclosure, and such carriers are well known in the art, including any of those described above.

In some embodiments, the non-viral DNA vectors may be formulated with a delivery vehicle. In some embodiments, the delivery vehicle is a lipid-based delivery vehicle. In some embodiments, the delivery vehicle is a lipid nanoparticle. As used herein, the term “lipid nanoparticle” or “LNP” refers to any lipid composition that can be used to deliver a therapeutic product, including, but not limited to, liposomes or vesicles, wherein an aqueous volume is encapsulated by amphipathic lipid bilayers, or wherein the lipids coat an interior that comprises a therapeutic product, or lipid aggregates or micelles, wherein the lipid-encapsulated therapeutic product is contained within a relatively disordered lipid mixture.

In some embodiments, lipid nanoparticles include lipid-based compositions with a solid lipid core stabilized by a surfactant. In some embodiments, the core lipids can be fatty acids, acyiglycerols, waxes, and mixtures of these surfactants. In some embodiments, biological membrane lipids such as phospholipids, sphingomyelins, bile salts (sodium taurocholate), and sterols (cholesterol) can be utilized as stabilizers.

In some embodiments, lipid nanoparticles can be formed using defined ratios of different lipid molecules, including, but not limited to, defined ratios of one or more cationic, anionic, or neutral lipids. In some embodiments, lipid nanoparticles can encapsulate molecules, such as the disclosed non-viral DNA vectors, within an outer-membrane shell and subsequently can be contacted with target cells to deliver the encapsulated molecules (e.g., the disclosed non-viral DNA vectors) to the host cell cytosol. In some embodiments, lipid nanoparticles can be modified or functionalized with non-lipid molecules, including on their surface (e.g., CD3, CD4, CD8, CD19, CD20, CD22, CD38, CD47, CDl 17, transferrin, ApoE, folate, etc.). In some embodiments, lipid nanoparticles can be modified to specifically bind to one or more receptors on the surface of the target cell (e.g., 1 or more receptors, 2 or more receptors, 3 or more receptors, 4 or more receptors, etc.). By “specifically bind” is meant that the lipid nanoparticle binds to the receptors on surface of the target cell with at least 2-fold greater affinity relative to the receptors on the surface of a non-target cell, e.g., at least 3-fold, 4-fold, 6-fold, 7-fold, 8-fold, 9-fold, 10-fold, 20-fold, 25-fold, 50-fold, or 100-fold higher. Cell surface receptors to which the modified lipid nanoparticles can bind include, but are not limited, to an integrin, transferrin receptor type land 2, EGF receptor, a VEGF receptor, an NGF receptor, CD3, CD4, CD7, CD8, CD19, CD20, CD22, CD33, CD43, CD38, CD56, CD69, the asialoglycoprotein receptor (ASGPR), N-acetyl-D-galactose (GalNAc) receptor, a folate receptor, and a sigma receptor. In some embodiments, the first and/or the second targeting ligand bind to the asialoglycoprotein receptor (ASGPR) or GalNAc receptor. Accordingly, in some embodiments, the modified lipid nanoparticles specifically bind to ASGPR or GalNAc receptor on surface of hepatocytes. In some embodiments, the targeting ligand used to modify the lipid nanoparticles is a carbohydrate or a carbohydrate conjugate. Carbohydrate based targeting ligands include, but are not limited to, glucose, multivalent glucose, fucose, D-mannose, multivalent mannose, lactose, multivalent lactose, D-galactose, multivalent galactose, GalNAc, multivalent GalNAc (e.g., GalNAc2 and GalNAc3), acetyl-galactosamine, N-acetyl-gulucosamine, glycosylated polyaminoacids and lectins. The term multivalent indicates that one, two, three or four monosaccharide units is present. Such monosaccharide subunits may be linked to each other through glycosidic linkages or linked to a scaffold molecule. In some embodiments, lipid nanoparticles can be single layered (unilamellar) or multi-layered (multilamellar). In some embodiments, lipid nanoparticles can be complexed with nucleic acid. Unilamellar lipid nanoparticles can be complexed with nucleic acid, wherein the nucleic acid is in the aqueous interior. In some embodiments, multilamellar lipid nanoparticles can be complexed with nucleic acid, wherein the nucleic acid is in the aqueous interior, or to form or sandwiched between.

In some embodiments, liposomal particles can, for example, be formed of a mixture of zwitterionic, cationic and anionic lipids which can be saturated or unsaturated, for example 1,2-distearoyl-sn-glycero-3-phosphocholine (DSPC) (zwitterionic, saturated), 1,2-dilinoleyoxy-3-dimethylaminopropane (DlinDMA) (cationic, unsaturated), and/or 1,2-dimyristoyl-rac-glycerol (DMG) (anionic, saturated). In some embodiments, the liposomes will typically comprise helper lipids. Useful helper lipids include zwitterionic lipids, such as DPPC, DOPC, DSPC, dodecylphosphocholine, 1,2-dioleoyl-sn-glycero-3-phosphatidylethanolamine (DOPE), and 1,2-diphytanoyl-sn-glycero-3-phosphoethanolamine (DPyPE); sterols, such as cholesterol; and PEGylated lipids, such as PEG-DMPE (PEG-conjugated 1, 2-dimyristoyl-Sn-glycero-3-phosphoethanolamine-N-[methoxy (polyethylene glycol)]) or PEG-DMG (PEG-conjugated 1,2-Dimyristoyl-sn-glycerol, methoxypolyethylene Glycol). In some embodiments, suitable PEGylated lipids include PEG2K-DMPE (PEG-conjugated 1, 2-dimyristoyl-Sn-glycero-3-phosphoethanolamine-N-[methoxy (polyethylene glycol)-2000]) or PEG2K-DMG (PEG-conjugated 1,2-Dimyristoyl-sn-glycerol, methoxypolyethylene Glycol-2000). In some embodiments, LNPs for use with the non-viral DNA vectors of the present disclosure include a zwitterionic lipid which can form liposomes, optionally in combination with at least one cationic lipid (such as N-[1-(2,3-Dioleoyloxy)propyl]-N,N,N-trimethylammonium methyl-sulfate (DOTAPBis(2-methacryloyl)oxyethyl disulfide (DSDMA), 2,3-Dioleyloxy-1-(dimethylamino)propane (DODMA), 1,2-dilinoleyoxy-3-dimethylaminopropane (DLinDMA), N,N-dimethyl-3-aminopropane (DLenDMA), etc.).

In some embodiments, the lipid nanoparticles have a mean diameter ranging from between about 20 nm to about 300 nm, e.g., from between about 20 nm to about 250 nm from between about 30 nm to about 200 nm, from between about 40 nm to about 180 nm, from between about 50 nm to about 150 nm, from between about 60 nm to about 140 nm, etc. Lipid nanoparticle particle size can be determined by quasi-elastic light scattering using, for example, a Malvern Zetasizer Nano ZS (Malvern, UK) system or electron microscope using, for example, FEI Quanta 200 Scanning Electron Microscope or FEI Tecnai Twin 120 kV Transmission Electron Microscope.

Examples of LNPs are described by Schoeenmaker et. al., “mRNA-lipid nanoparticle COVID-19 vaccines: Structure and stability,” Int J Pharm. 2021 May 15; 601: 120586, the disclosure of which is hereby incorporated by reference herein in its entirety. Other exemplary LNPs are described by Eygeris et. al., “Chemistry of Lipid Nanoparticles for RNA Delivery,” Ace Chem Res. 2022 Jan. 4; 55(1):2-12. doi: 10.1021/acs.accounts.1c00544. Epub 2021 Dec. 1. PMID: 34850635, the disclosure of which is hereby incorporated by reference herein in its entirety. Yet other suitable LNPs for use with the non-viral DNA vectors of the present disclosure are described in United States Patent Publication Nos. 2021/0371877, 2022/0175968, 2022/0042035, and 2022/0062409, the disclosures of which are hereby incorporated by reference herein in their entireties.

The non-viral DNA vectors of the present disclosure may also be formulated with one or more polymers. Various polymers or copolymers may be adapted as a vehicle for the non-viral DNA vectors of the present disclosure. Exemplary polymeric materials include poly(D,L-lactic acid -co-glycolic acid) (PLGA), poly(caprolactone) (PCL), ethylene vinyl acetate polymer (EVA), poly(lactic acid) (PLA), poly(L-lactic acid) (PLLA), poly(glycolic acid) (PGA), poly(L-lactic acid -co-glycolic acid) (PLLGA), poly(D,L-lactide) (PDLA), poly(L-lactide) (PLLA), PLGA-b-poly(ethylene glycol)-PLGA (PLGA-bPEG-PLGA), PLLA-bPEG-PLLA, PLGA-PEG-maleimide (PLGA-PEG-mal), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) (polyacrylic acids), and copolymers and mixtures thereof, polydioxanone and its copolymers, polyhydroxyalkanoates, polypropylene fumarate), polyoxymethylene, poloxamers, poly(ortho)esters, poly(butyric acid), poly(valeric acid), poly(lactide-co-caprolactone), trimethylene carbonate, polyvinylpyrrolidone, polyorthoesters, polyphosphazenes, Poly([beta]-amino esters (PBAE), and polyphosphoesters, and blends and/or block copolymers of two or more such polymers. Polymer-based systems may also include Cyclodextrin polymer (CDP)-based nanoparticles such as, for example, CDP-admantane (AD)-PEG conjugates and CDP-AD-PEG-transferrin conjugates.

Non-limiting examples of polymeric particle systems for delivery of the disclosed non-viral DNA vectors include the systems described in U.S. Pat. Nos. 5,543,158, 6,007,845, 6,254,890, 6,998,115, 7,727,969, 7,427,394, 8,323,698, 8,071,082, 8,105,652, US 2008/0268063, us 2009/0298710, us 2010/0303723, us 2011/0027172, us 2011/0065807, us 2012/0156135, US 2014/0093575, WO 2013/090861, the disclosures of which are hereby incorporated by reference herein in their entireties.

In some embodiments, the non-viral DNA vectors may be formulated as pharmaceutically acceptable nanocapsule formulations. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention. Such particles may be easily made, as described (Couvreur et al., 1980; Couvreur, 1988; zur Muhlen et al., 1998; Zambaux et al. 1998; Pinto-Alphandry et al., 1995 and U.S. Pat. No. 5,145,684, specifically incorporated herein by reference in its entirety).

In some embodiments, the pharmaceutical compositions including the non-viral DNA vectors of the present disclosure, and which are suitable for injectable use include sterile aqueous solutions or dispersions and sterile powders for the extemporaneous preparation of sterile injectable solutions or dispersions (see U.S. Pat. No. 5,466,468, the disclosure of which is hereby incorporated by reference herein in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils.

In some embodiments, the non-viral DNA vectors may be given to the patients with physical methods, such as electroporation, sonoporation with microbubbles, sonoporation without microbubbles, magnetofection, hydroporation, photoporation, mechanical massage, jet injection, biolistics (gene gun), hydrodynamic injection, needle injection or microinjections.

### Methods of Treatment

The present disclosure is also directed to administering therapeutically effective amounts of a circular, non-viral DNA vector capable of expressing one or more transgenes or a pharmaceutical composition comprising a circular, non-viral DNA vector capable of expressing one or more transgenes to a patient in need of treatment thereof.

In some embodiments, the present disclosure is directed to treating a condition or disease related to a bone defect characterized by a lack of or an insufficient amount of functional alkaline phosphatase. Another aspect of the present disclosure is directed to a method of treating hypophosphatasia in a mammal, e.g., a human, in need thereof. Another aspect of the present disclosure is directed to a method of treating, mitigating, or preventing a symptom of hypophosphatasia in a mammal, e.g., a human.

Hypophosphatasia (HPP) is a rare, heritable skeletal disease with an incidence of 1 per 100,000 births for the most severe forms of the disease. The disorder results from loss-of-function mutations in the gene encoding tissue-nonspecific alkaline phosphatase (TNALP). HPP patients present a remarkable range of symptoms, from teeth loss or osteomalacia (rickets) to almost complete absence of bone mineralization in utero. Many patients with HPP present the characteristics of skeletal deformities, short stature, muscle and bone pain, impaired mobility, and premature loss of teeth. Perinatal-onset or infantile-onset HPP can also be characterized by the presence of rachitic chest deformity, vitamin B6-dependent seizures, and failure to thrive. In particular, HPP presenting at less than six months of age is often lethal due to respiratory insufficiency, with a low survival rate at one year of age.

In some embodiments, the methods of the present disclosure provide for the treatment of hypophosphatasia in the mammal. As used herein, the terms “treatment,” “treating,” and the like refer to obtaining a desired pharmacologic and/or physiologic effect. In some embodiments, the effect is therapeutic, i.e., the effect partially or completely cures a disease and/or adverse symptom attributable to the disease. In some embodiments, a “therapeutically effective amount” of the composition comprising the non-viral DNA vector are administered to the subject in need of treatment thereof. A “therapeutically effective amount” refers to an amount effective, at dosages and for periods of time necessary, to achieve a desired therapeutic result.

In some embodiments, hypophosphatasia may be treated by administering a therapeutically effective amount of a pharmaceutical composition including a circular, non-viral DNA vector including one or more nucleic acid sequences encoding TNALP or a polypeptide having an amino acid sequence encoding TNALP (see, e.g., SEQ ID NOS: 28-30, 38, 40-48, and 72-73). In other embodiments, treating, mitigating, or preventing a symptom of hypophosphatasia in a mammal comprises administering a therapeutically effective amount of a pharmaceutical composition including a circular, non-viral DNA vector including one or more nucleic acid sequences encoding TNALP or a polypeptide having an amino acid sequence encoding TNALP (see, e.g., SEQ ID NOS: 28-30, 38, 40-48, and 72-73).

In some embodiments, the therapeutically effective amount may vary according to factors such as the disease state, age, sex, and weight of the individual.

The methods provided herein may be carried out by administering the pharmaceutical compositions by any suitable routes of administration. The route of administration can be local or systemic. Exemplary routes of administration include, for example, the nasal, pulmonary, inhalation, intraarterial, intradermal, intralesional, intramuscular, intraperitoneal, intravenous, intrathecal, intravesical, parenteral, rectal, subcutaneous, and transmucosal. In some embodiments, the non-viral DNA vectors are administered in suitably formulated pharmaceutical compositions disclosed herein either subcutaneously, intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection. The methods of administration may also include those modalities as described in U.S. Pat. Nos. 5,543,158; 5,641,515 and 5,399,363, each of which are incorporated by reference herein in their entireties.

In some embodiments, a subject in need of treatment is treated over a particular duration of time. In some embodiments, the duration of treatment is from about 1 week to about 10 years. In some embodiments, the duration of treatment is from about 1 week to about 5 years. In some embodiments, the duration of treatment is from about 1 week to about 1 year. In some embodiments, the duration of treatment is from about 1 week to about 6 months. In some embodiments, the duration of treatment is from about 1 week to about 3 months. In some embodiments, the duration of treatment is from about 1 week to about 1 month. In some embodiments, the duration of treatment is from about 3 months to about 5 years. In some embodiments, the duration of treatment is from about 6 months to about 5 years. In some embodiments, the duration of treatment is from about 1 year to about 5 years.

Each dose may be administered over any suitable period of time. In some embodiments, the dose is administered as a bolus dose. In some embodiments, the dose is administered over a period of about 1 minute to about 4 hours. In some embodiments, the dose is administered over a period of about 1 minute to about 2 hours. In some embodiments, the dose is administered over a period of about 1 minute to about 1 hour. In some embodiments, the dose is administered over a period of about 1 minute to about 30 minutes. In some embodiments, the dose is administered over a period of about 1 minute to about 15 minutes.

As noted herein, the circular, non-viral DNA vectors are amenable to redosing. In some embodiments, the circular, non-viral DNA vectors are redosed for a time period ranging from between about two weeks to about five years. In some embodiments, the pharmaceutical composition is administered according to a particular frequency. In some embodiments, the frequency is daily, every 2 days, every 3 days, every 4 days, every 5 days, every 6 days, every 7 days, every 8 days, every 9 days, every 10 days, every 11 days, every 12 days, every 13 days, or every 14 days. In some embodiments, the frequency is every 3 weeks, every 4 weeks, every 5 weeks, every 6 weeks, every 7 weeks, every 8 weeks, every 9 weeks, every 10 weeks, every 11 weeks, or every 12 weeks. In some embodiments, the frequency is every 1 month, every 2 months, every 3 months, every 4 months, every 5 months, every 6 months, every 7 months, every 8 months, every 9 months, every 10 months, every 11 months, every 12 months, every 13 months, every 14 months, every 15 months, every 16 months, every 17 months, or every 18 months. In some embodiments, the frequency is every 2 years, every 3 years, every 4 years, or every 5 years.

### EXAMPLES

### Example 1—In Vitro Resolution of a Reporter Non-Viral DNA Construct in IPSC-Derived Hepatocytes

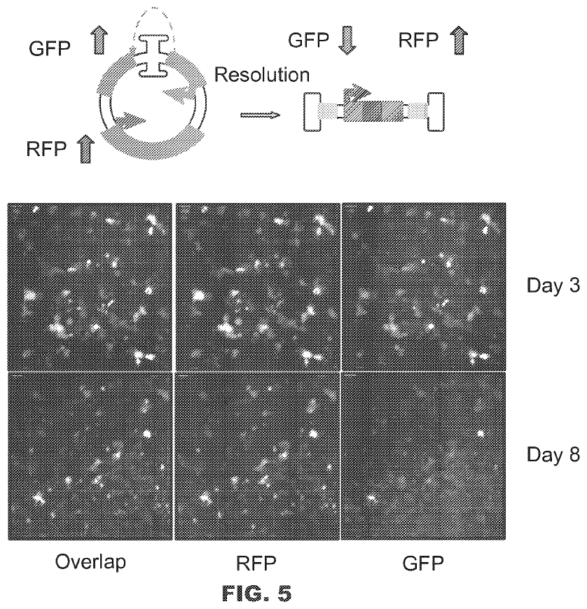

Non-dividing iPSC-derived hepatocytes transfected with a circular, non-viral DNA vector of the present disclosure including an RFP/GFP reporter (SEQ ID NO: 32) showed simultaneous GFP and RFP expression at day 3 and mainly RFP at day 8 in FIG. 5. The data suggested that the circular, non-viral DNA vector containing a cruciform structure could be resolved into linear form in non-dividing primary human iPSC-derived hepatocytes.

A reporter system was developed for assessing resolution of a circular, non-viral DNA vector to a linear form once introduced into a mammalian cell. The circular molecule would express both a red fluorescent protein (RFP) and a green fluorescent protein (GFP) while GFP expression in the circular form proceeded via splicing the mRNA across the cruciform DNA structure would be interrupted and the linearized molecule would only express the RFP.

### Method

Non-dividing iPSC-derived human hepatocytes were purchased from Fujifilm. At day 0, plating medium was prepared by adding 1.5 mL of B27 supplement, 0.15 mL of 10 μg/mL of oncostatin stock solution, 1.5 μL of 5 mM of dexamethasone stock solution, 37.5 μL of 25 μg/mL of gentamicin and 1.5 mL of iCell Hepatocytes 2.0 medium supplement into 72 mL of RPMI medium according to Fujifilm's recommendation.

Plating medium was filtered via 0.22 μm PES filter unit. At day 1, the iCell Hepatocytes 2.0 cryovial was removed from the liquid nitrogen storage tank. The cryovial was immersed in a 37° C. water bath for exactly 3 minutes. Using a 2 mL serological pipette, the iCell Hepatocytes 2.0 cryovial contents was gently transferred into the 15 mL centrifuge tube containing 10 mL of 37° C. Plating Medium. The cell suspension was centrifuged at 200×g for 3 minutes at room temperature. The supernatant was carefully aspirated under vacuum. 2 mL of 37° C. Plating Medium was slowly added with a wide-bore pipette to resuspend the cell pellet. Hepatocytes were seeded in pre-warmed collagen-coated 24/48-well plate. The medium was refreshed daily from day 1 to 7 and every other day starting from day 8.

TransIT-LTl Reagent and non-viral vector DNA complex was prepared following Mirus manufacturer's recommendation. The TransIT-LT1 Reagent:DNA complex was added dropwise to different areas of the wells. The culture vessel was gently rocked back-and-forth and from side-to-side to evenly distribute the TransIT-LTl Reagent:DNA complexes.

### Results

FIG. 5 demonstrated that iPSC-derived hepatocytes transfected with a circular, non-viral DNA reporter vector of the present disclosure including an RFP/GFP reporter (SEQ ID NO: 32) showed simultaneous GFP and RFP expression at day 3 but mainly RFP only at day 8. The data suggested that the circular, non-viral DNA vector containing a cruciform structure could be resolved into linear form in non-dividing primary human iPSC-derived hepatocytes.

### Example 2—In Vitro Durable Gene Expression of Firefly Luciferase DNA Construct in IPSC-Derived Hepatocytes

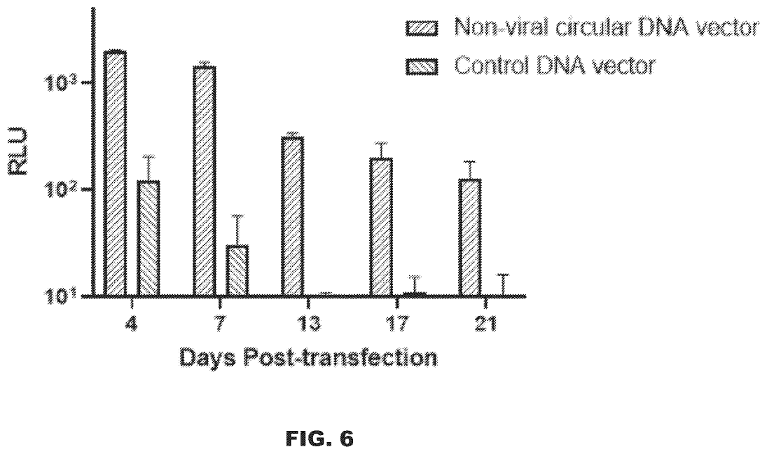

Transfection of a circular, non-viral DNA vector of the present disclosure encoding TNALP (M014—SEQ ID NO: 30) led to strong bioluminescent emission in iPSC-derived hepatocytes from day 3; and this signal was maintained from day 12 to day 21 while Relative Luminescence Unit (RLU) from cells transfected with a control circular, non-viral DNA vector of the present disclosure encoding TNALP (P021—SEQ ID NO: 31) dropped significantly from day 7 shown in FIG. 6. The circular, non-viral DNA vectors of the present disclosure including a luciferase reporter gene showed robust and durable luciferase expression whereas the same control construct carrying antibiotics-resistance gene were far less potent and did not last.

### Method

Non-dividing iPSC-derived human hepatocytes were purchased from Fujifilm. At day 0, plating medium was prepared by adding 1.5 mL of B27 supplement, 0.15 mL of 10 μg/mL of oncostatin stock solution, 1.5 μL of 5 mM of dexamethasone stock solution, 37.5 μL of 25 μg/mL of gentamicin and 1.5 mL of iCell Hepatocytes 2.0 medium supplement into 72 mL of RPMI medium according to Fujifilm's recommendation.

Plating medium was filtered via 0.22 μm PES filter unit. At day 1, the iCell Hepatocytes 2.0 cryovial was removed from the liquid nitrogen storage tank. The cryovial was immersed in a 37° C. water bath for exactly 3 minutes. Using a 2 mL serological pipette, the iCell Hepatocytes 2.0 cryovial contents was gently transferred into the 15 mL centrifuge tube containing 10 mL of 37° C. Plating Medium. The cell suspension was centrifuged at 200×g for 3 minutes at room temperature. The supernatant was carefully aspirated under vacuum. 2 mL of 37° C. Plating Medium was slowly added with a wide-bore pipette to resuspend the cell pellet. Hepatocytes were seeded in pre-warmed collagen-coated 24/48-well plate. The medium was refreshed daily from day 1 to 7 and every other day starting from day 8.

TransIT-LT1 Reagent and non-viral vector DNA complex was prepared following Mirus manufacturer's recommendation. The TransIT-LT1 Reagent:DNA complex was added dropwise to different areas of the wells. The culture vessel was gently rocked back-and-forth and from side-to-side to evenly distribute the TransIT-LTl Reagent:DNA complexes.

For non-invasive bioluminescent readout, luciferin substrate stock solution was added into the cell plate to reach 5 mM final concentration then RLU from each well of the plate was measured by Molecular Device plate reader iD5.

### Example 3—In Vitro Durable Gene Expression of TNALP DNA Constructs in IPSC-Derived Hepatocytes

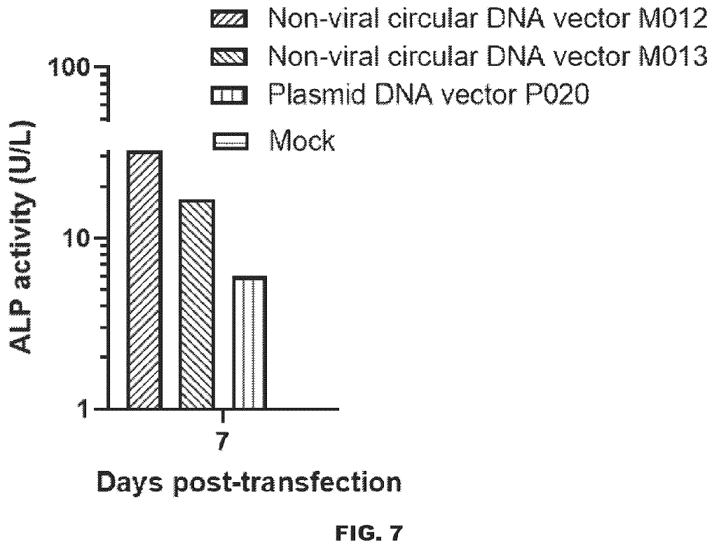

Non-dividing iPSC-derived hepatocytes transfected with circular, non-viral DNA vectors of the present disclosure including a nucleic acid encoding TNALP and also including dual inverted repeats (M012—SEQ ID NO: 28) showed higher ALP secretion as compared with hepatocytes transfected with a circular, non-viral DNA vector including a nucleic acid encoding TNALP and also including a series of single inverted repeats without intervening heterologous sequences (M013—SEQ ID NO: 29) in FIG. 7. Cells transfected with both constructs comprising the inverted repeat sequences showed higher ALP secretion than cells transfected with a control TNALP construct comprising ampicillin resistance gene (P020—SEQ ID NO: 33).

### Method

Non-dividing iPSC-derived human hepatocytes were purchased from Fujifilm. At day 0, plating medium was prepared by adding 1.5 mL of B27 supplement, 0.15 mL of 10 μg/mL of oncostatin stock solution, 1.5 μL of 5 mM of dexamethasone stock solution, 37.5 μL of 25 μg/mL of gentamicin and 1.5 mL of iCell Hepatocytes 2.0 medium supplement into 72 mL of RPMI medium according to Fujifilm's recommendation.

Plating medium was filtered via 0.22 μm PES filter unit. At day 1, the iCell Hepatocytes 2.0 cryovial was removed from the liquid nitrogen storage tank the cryovial was immersed in a 37° C. water bath for exactly 3 minutes. Using a 2 mL serological pipette, the iCell Hepatocytes 2.0 cryovial contents was gently transferred into the 15 mL centrifuge tube containing 10 mL of 37° C. Plating Medium. The cell suspension was centrifuged at 200×g for 3 minutes at room temperature. The supernatant was carefully aspirated under vacuum. 2 mL of 37° C. Plating Medium was slowly added with a wide-bore pipette to resuspend the cell pellet. Hepatocytes were seeded in pre-warmed collagen-coated 24/48-well plate. The medium was refreshed daily from day 1 to 7 and every other day starting from day 8.

TransIT-LTl Reagent and non-viral vector DNA complex was prepared following Mirus manufacturer's recommendation. The TransIT-LT1 Reagent:DNA complex was added dropwise to different areas of the wells. The culture vessel was gently rocked back-and-forth and from side-to-side to evenly distribute the TransIT-LTl Reagent:DNA complexes.

At different timepoints, the cell culture medium was taken out and ALP activity in the cell culture medium was determined by ALP colorimetric assay carried out on a Molecular Device plate reader iD5.

### Example 4—Persistence of Liver Luciferase Expression from Luciferase-Containing Constructs in Adult Mice

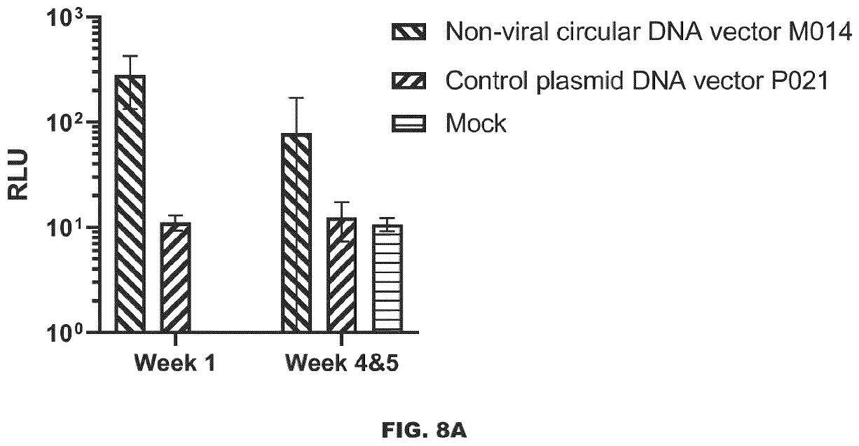

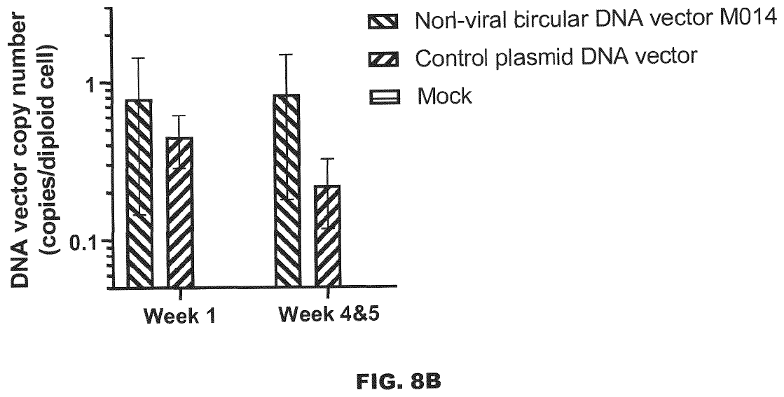

A circular, non-viral DNA vector encoding a luciferase reporter (M014—SEQ ID NO: 30) in accordance with the present disclosure showed persistent bioluminescent signal from mouse liver tissue harvested from week 1 to weeks 4 and 5 after dosing through hydrodynamic tail vein injection (15 μg per mouse) while mice injected with control luciferase plasmid showed background level of luminescence shown in FIG. 8A. Further DNA copy number per diploid cell analysis showed that the M014 construct was maintained for one month in mouse liver tissue while the copy number of a control luciferase plasmid (P021—SEQ ID NO: 31) in mouse liver cells dropped significantly from week 1 to week 5 shown in FIG. 8B.

### Method

Adult mice (strain: BALB/c) at age of 9-11 weeks were ordered from The Jackson Laboratory. Prior to study start, animals were randomized into groups using a random number generator. Non-viral DNA vector constructs were administered to adult mice by hydrodynamic injection through the tail vein at dose of 15 μg per animal and volume of 80 mL/kg. At several terminal timepoints, luciferase activity and the non-viral DNA vector copy number were measured ex-vivo in the liver tissues.

For constructs according to the present disclosure DNA copy number measurement, about 25 mg of each liver lobe from each animal were collected and stored below −65° C. before ddPCR assay was carried out on a Biorad ddPCR system. Primers/probe targeting polyA region of non-viral DNA vector construct were used to measure non-viral DNA vector construct copy number while mouse RPP30 was used as a reference gene.

For luciferase, the remaining tissue from each liver lobe was stored at below −65° C. for lysate generation/luciferase. Spectra Max Glo Steady-Luc Reporter Assay Kit was used to measure bioluminescence from the liver lysate after homogenization.

### Example 5—Comparison of Alp Levels in Mice Treated with a Circular, Non-Viral DNA Vector Expressing TNALP and a Lentiviral Vector Expressing TNALP

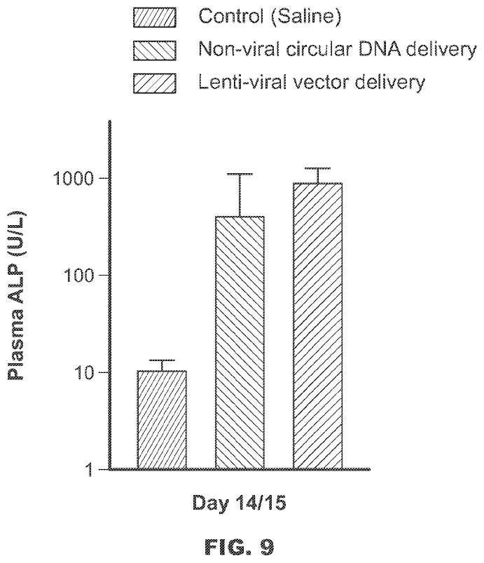

FIG. 9 shows that similar plasma ALP levels were found in mice (n=3) dosed with a circular, non-viral DNA vector including a nucleic acid encoding TNALP (M013—SEQ ID NO: 29) as compared with mice treated with 1×106 cells transduced with a lentiviral vector expressing TNALP (SEQ TD NO: 34) (transduced hematopoietic stem/progenitor cells at 2 weeks post-dosing).

### Method

Adult mice (strain: B6129SF2/J) at age of 9-11 weeks were ordered from The Jackson Laboratory. Prior to study start, animals were randomized into groups using a random number generator. Non-viral DNA vector constructs were administered to adult mice by hydrodynamic injection through the tail vein at dose of 15 μg per animal and volume of 80 mL/kg. At several terminal timepoints, luciferase activity and non-viral DNA vector copy number were measured in the liver.

For DNA vector copy number measurement, 25 mg of each liver lobe from each animal were collected and stored below −65° C. before ddPCR assay was carried out on a Biorad ddPCR system. Primers/probe targeting the polyA region of the non-viral DNA vector construct were used to measure non-viral DNA vector construct copy number while mouse RPP30 was used as a reference gene.

At different timepoints, plasma samples were collected from the animals and stored below −65° C. before ALP measurement was carried out. ALP concentration was determined by colorimetric assay carried out on a Molecular Device plate reader iD5.

### Example 6—Production of Construct According to the Present Disclosure

In some embodiments, a circular, non-viral DNA vector can be generated from a parental plasmid including both a marker gene and high copy number origin of replication (e.g., pUC or pMBl) between two loxP sites by “Cre-lox recombination.” The Cre recombinase can be induced through metabolic control in the bacterial cells harboring the parental plasmid. In this instance, recombination produces two circular and supercoiled DNA molecules which are topologically unlinked, each containing a single loxP site. The one resulting circular supercoiled DNA molecule comprises the marker gene and the high copy number origin of replication, while the other comprises the circular non-viral DNA vector of this disclosure.

In some embodiments, other LoxP sites such as Lox511, Lox 5171, Lox 2272, M2, M3, M7, M11, Lox 71, Lox 66, LoxPsym, or others could be used to generate the non-viral DNA vector from a parental DNA plasmid. In some embodiments, other recombinases such as PhiC31, A integrase, and Flp recombinase could be used to generate the non-viral DNA vector. In some embodiments, recombinases such as Cre, PhiC31, A integrase, and Flp recombinase could be produced recombinantly and applied directly to purified parental plasmid DNA to generate the non-viral DNA vector. Tn some embodiments, marker genes would include those the encode kanamycin resistance, spectinomycin resistance, streptomycin resistance, carbenicillin resistance, bleomycin resistance, erythromycin resistance, polymyxin B resistance, tetracycline resistance, or chloramphenicol resistance among others. In some embodiments, origins of replication would include those sequences from pMB, pBR322, Co1E1, p15A, pSC101, or F1.

### Example 7—Persistence of Liver Murine SEAP Expression from Murine SEAP Constructs in Adult Mice

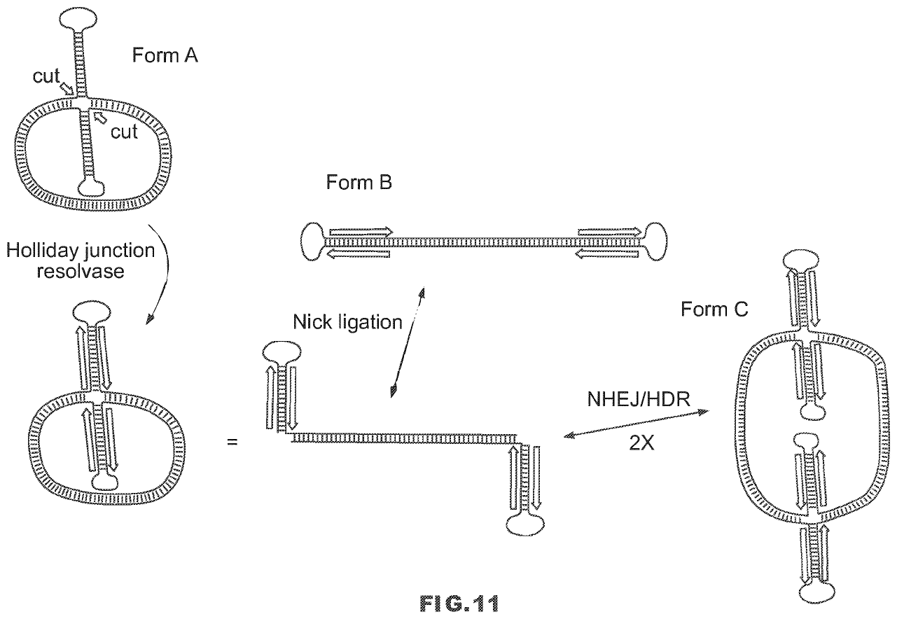

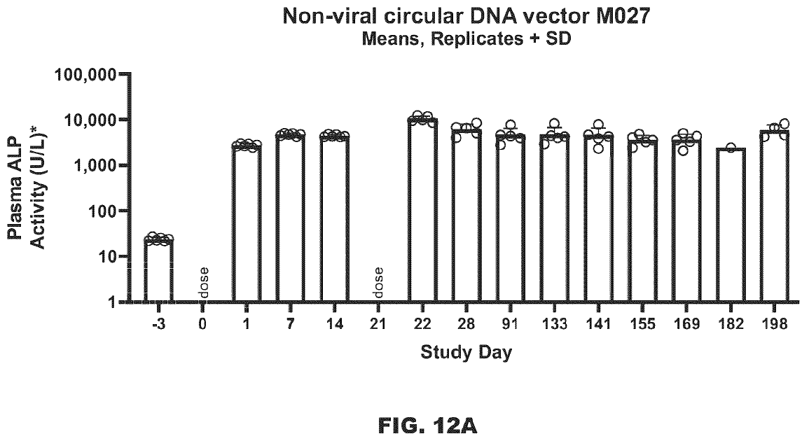

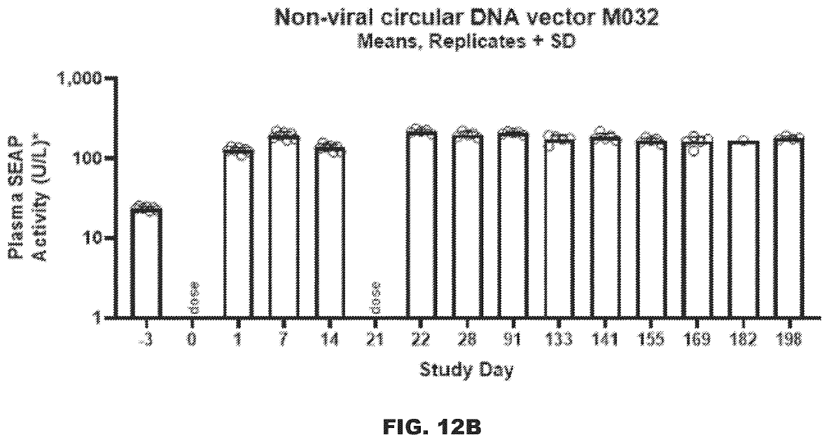

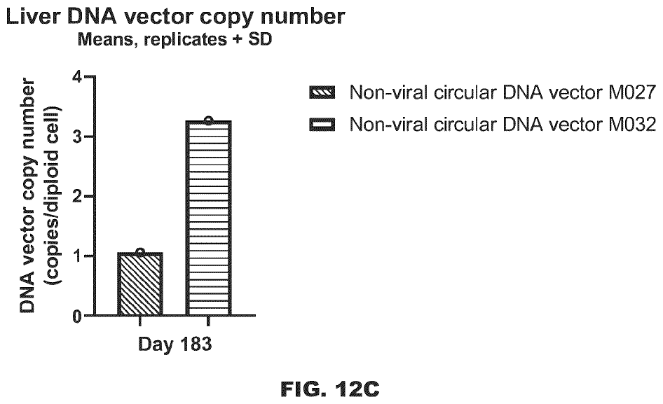

Circular, non-viral DNA vectors using EFla and TBG promoters encoding a murine SEAP reporter (M027 and M032—SEQ ID NO: 74 and SEQ ID NO: 75) showed persistence of high plasma SEAP activity in mouse samples collected from day 1 to day 190 after dosing through hydrodynamic tail vein injection (15 μg per mouse) (FIGS. 12A and 12B), respectively. DNA copy number per diploid cell analysis showed that the two constructs according to the present disclosure (M027—SEQ ID NO: 74; and M032—SEQ ID NO: 75) were maintained for 189 days in mouse liver (FIG. 12C).

### Method

Adult mice (strain: BALB/c) at age of 9-11 weeks were ordered from The Charles River Laboratories. Prior to study start, animals were randomized into groups using a random number generator. Non-viral DNA vector constructs were administered to adult mice by hydrodynamic injection through the tail vein at dose of 15 μg per animal and volume of 100 mL/kg. At several tem1inal timepoints, SEAP activity were measured in plasma samples and the non-viral DNA vector copy number was measured in liver tissue samples.

For constructs according to the present disclosure, copy number measurement, 25 mg of each liver lobe from each animal were collected and stored below −65° C. before ddPCR assay was carried out on a BioRad ddPCR system. Primers/probe targeting the polyA region of a reference gene.

At different timepoints, plasma samples were collected and stored below −65° C. before the SEAP assay was carried out. SEAP activity was determined by SEAP fluorescence assays carried out on a Molecular Device plate reader iD5.

### Example 8—Nuclear Entry of Constructs According to the Present Disclosure

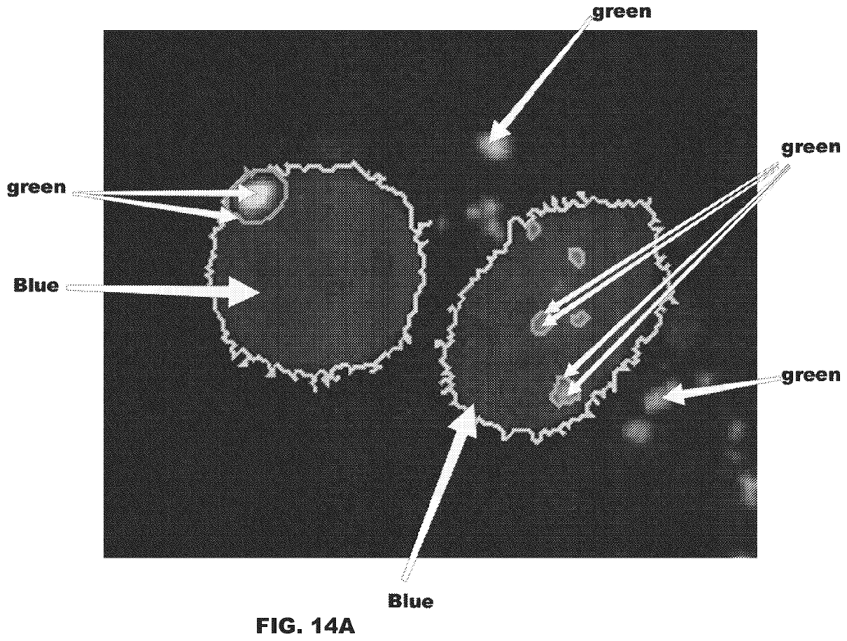

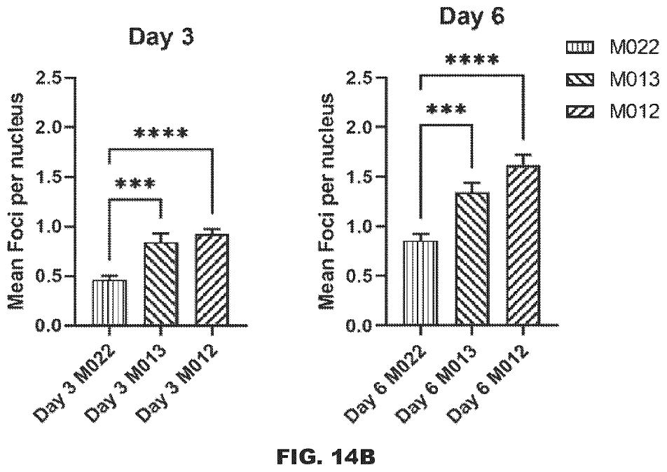

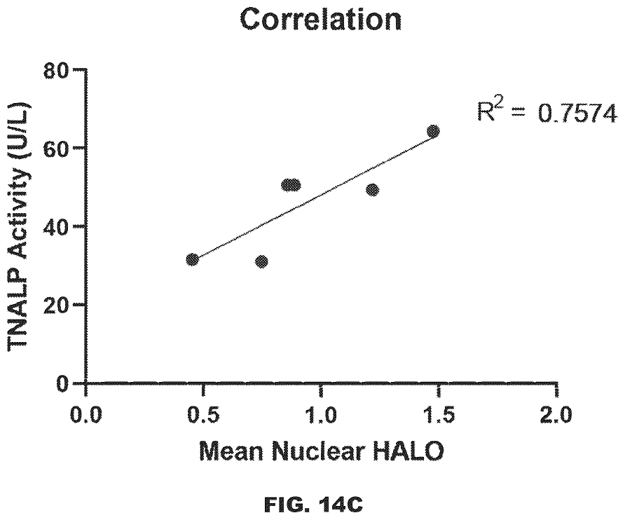

To determine the extent to which the cruciform structure impacts nuclear entry and retention, post-mitotic non-dividing iPSC-derived hepatocytes transfected with fluorescence-labeled circular, non-viral DNA vectors including a nucleic acid encoding TNALP and also including dual inverted repeats (M012—SEQ ID NO: 28) showed higher number of foci per nucleus as compared with hepatocytes transfected with a circular, non-viral DNA vector including a nucleic acid encoding TNALP and also including a series of single inverted repeats without intervening heterologous sequences (DD-ITR construct) (M013—SEQ ID NO: 29) and a control TNALP construct comprising ampicillin resistance gene (M022—SEQ ID NO: 33) at day 1, 3 and 6 (FIG. 14B). The number of foci per nucleus correlates with ALP activity of medium (FIG. 14C).

### Method

Non-dividing iPSC-derived human hepatocytes were purchased from Fujifilm. At day 0, plating medium was prepared by adding 1.5 mL of B27 supplement, 0.15 mL of 10 μg/mL of oncostatin stock solution, 1.5 uL of 5 mM of dexamethasone stock solution, 37.5 μL of 25 μg/mL of gentamicin and 1.5 mL of iCell Hepatocytes 2.0 medium supplement into 72 mL of RPMI according to Fujifilm's recommendation.

After iCell Hepatocytes 2.0 Medium Supplement (1×3.0 mL) was thawed, plating medium was filtered via 0.22 μm PES filter unit. At day 1, the iCell Hepatocytes 2.0 cryovial was taken out from the liquid nitrogen storage tank. The cryovial was immersed in a 37° C. water bath for exactly 3 minutes. Using a 2 mL serological pipette, the iCell Hepatocytes 2.0 cryovial contents were gently transferred into the 15 mL centrifuge tube containing 10 mL of 37° C. Plating Medium. The cell suspension was centrifuged at 200×g for 3 minutes at room temperature. After the supernatant was carefully aspirated, 2 mL of warm plating medium with a wide-bore pipette was used to resuspend the cell pellet. Hepatocytes were seeded in pre-warmed collagen-coated 24/48-well plate.

Transfection Reagent was purchased from FuGENE and DNA transfection followed manufacturer's recommendation.

At different timepoints, the cell culture medium was taken out and ALP level was determined by ALP colorimetric assay carried out on a Molecular Device plate reader iD5.

Cells were fixed on day 1, 3, and 6 post-transfection, stained with DAPI, and mounted for confocal images. DAPI staining (blue) represents the nucleus and green foci represent the constructs of the present disclosure inside the cell, both in the nucleus and cytoplasm (FIG. 14A).

Images were analyzed using ImagePro 11 smart segmentation. Nuclei regions of interest (ROIs) were identified on the blue channel using a minimum area cut-off of 58 μm². ROIs including the constructs of the present disclosure were identified on the green channel using a minimum area cut-off of 0.04 μm². Only foci including constructs of the present disclosure located within nuclei were counted.

An average of 400 cells were analyzed per condition. Data are graphed as the average number of foci per nucleus for each condition. Outliers were excluded from the dataset using 3 criteria (nuclei area, # of foci, and foci % area of nuclei).

### Example 9—In Vitro Gene Expression of Reporter Constructs with Different Cruciforvi Structures in IPSC-Derived Hepatocytes

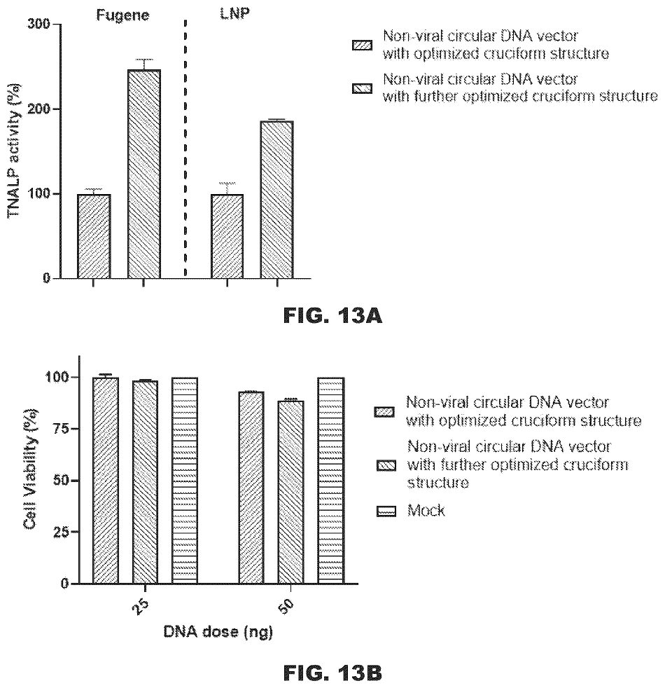

FIG. 13A shows that transfection of a circular non-viral DNA vector with further improved cruciform structure (M056—SEQ ID NO:73) led to stronger ALP level in culture medium in iPSC-derived hepatocytes at day 3 than a circular non-viral DNA vector with improved cruciform structure (M012—SEQ ID NO: 30) with FuGENE or SM102-based lipid nanoparticle formulation. Both of circular, non-viral DNA vectors with FuGENE or SM102-based lipid nanoparticle formulation do not induce significant toxicity in iPSC-derived human hepatocytes shown in FIG. 13B.

### Method

Non-dividing iPSC-derived human hepatocytes were purchased from Fujifilm. At day 0, plating medium was prepared by adding 1.5 mL of B27 supplement, 0.15 mL of 10 μg/mL of oncostatin stock solution, 1.5 μL of 5 mM of dexamethasone stock solution, 37.5 μL of 25 μg/mL of gentamicin and 1.5 mL of iCell Hepatocytes 2.0 medium supplement into 72 mL of RPMI according to Fujifilm's recommendation.

After iCell Hepatocytes 2.0 Medium Supplement (1×3.0 mL) was thawed, plating medium was filtered via 0.22 μm PES filter unit. At day 1, the iCell Hepatocytes 2.0 cryovial was taken out from the liquid nitrogen storage tank. The cryovial was immersed in a 37° C. water bath for exactly 3 minutes. Using a 2 mL serological pipette, the iCell Hepatocytes 2.0 cryovial contents were gently transferred into the 15 mL centrifuge tube containing 10 mL of 37° C. Plating Medium. The cell suspension was centrifuged at 200×g for 3 minutes at room temperature. After the supernatant was carefully aspirated, 2 mL of warm plating medium with a wide-bore pipette was used to resuspend the cell pellet. Hepatocytes were seeded in pre-warmed collagen-coated 24/48-well plate.

Prepare FuGene or SM-102-based lipid nanoparticle transfection Reagent and a non-viral vector DNA complex following supplier's recommendation. Add the transfection reagent:DNA complexes (prepared in Step A) dropwise to different areas of the wells. Gently rock the culture vessel back-and-forth and from side-to-side to evenly distribute the transfection reagent:DNA complexes.

At day 3, the cell culture medium was taken out and ALP level was determined by ALP colorimetric assay carried out on a Molecular Device plate reader iD5.

### Example 10—In Vitro Gene Expression of a Reporter Constructs with or without Cruciform Structure in Primary Human and Cynomolgus Monkey Hepatocytes

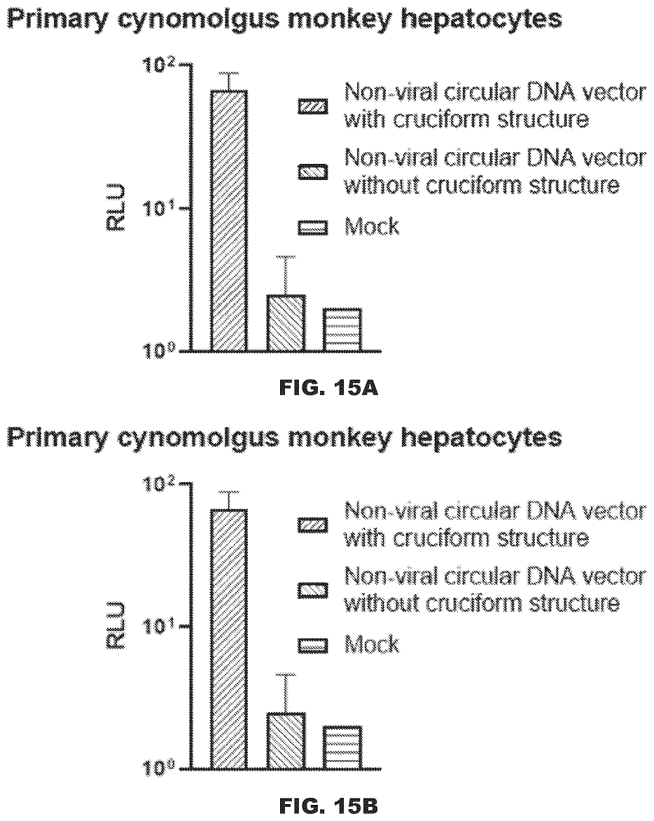

Transfection of constructs of the present disclosure which included luciferase DNA (SEQ ID NO: 73) with improved cruciform structure led to a much higher luciferase level in primary human hepatocytes (FIG. 15A) and cynomolgus monkey hepatocytes (FIG. 15B) at day 3 than control luciferase DNA without cruciform structure (SEQ ID NO: 74) using SM102-based lipid nanoparticle formulation.

### Method

Primary human hepatocytes (Lonza) and primary cynomolgus monkey hepatocytes (GIBCO) were seeded on day 0 in duplicate wells of collagen type I-coated 96 well plates at 80,000 and 70,000 cells per well. One day post seeding, constructs of the present disclosure and control constructs were delivered by LNP mix.

Prepare SM-102-based lipid mix (SM102, DSPC, NIP 6, 1.5% DMG-PEG2K) and use pipette to mix DNA with lipid mix. Add lipid mix:DNA complexes dropwise to different areas of the wells. Gently rock the culture vessel back-and-forth and from side-to-side to evenly distribute lipid DNA mix.

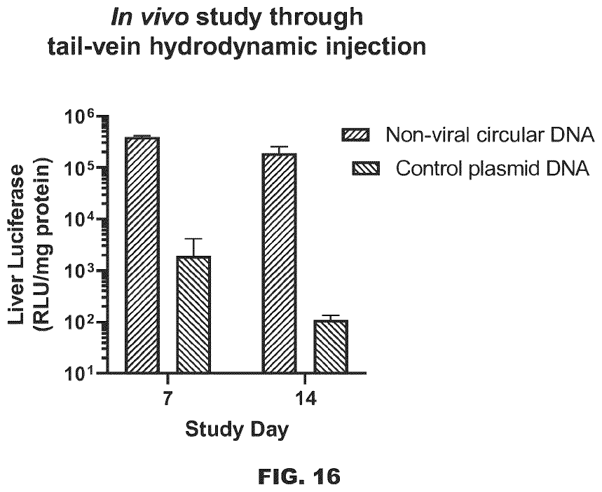

Endpoint luciferase activity was measured in cell lysate of primary human hepatocytes (FIG. 16A) and primary cynomolgus monkey hepatocytes (FIG. 16B) on day 6 post LNP delivery with Molecular Devices Glo Steady-Luc kit, respectively.

### Example 11—Enhanced Liver Luciferase Expression from Firefly Luciferase Constructs in Adult Mice

A circular, non-viral DNA vector including a firefly luciferase reporter (SEQ ID NO: 73) showed higher expression of luciferase activity in mouse liver tissue harvested from day 7 and 14 after dosing through tail vein-based hydrodynamic injection (15 μg per mouse) compared to a control firefly luciferase construct (SEQ ID NO: 73) (FIG. 16).

### Method

Adult mice (strain: BALB/c) at age of 9-11 weeks were ordered from The Charles River Laboratories. Prior to study start, animals were randomized into groups using a random number generator. Non-viral DNA vector constructs were administered to adult mice by hydrodynamic injection through the tail vein at dose of 15 μg per animal and volume of 100 mL/kg. On day 7 and 14, firefly luciferase activity was measured in liver samples.

At day 7 and day 14, the mice were euthanized, and liver samples were collected and stored at below −65° C. before bioluminescent measurement was determined by a bioluminescence assay carried out on a Molecular Device plate reader iD5.

### Example 12—In Vitro Gene Expression Comparison of DNA Construct and Chemically Modified mRNA Encoding a Cynomolgus Monkey Soluble TNALP Transgene in Primary Human Hepatocytes

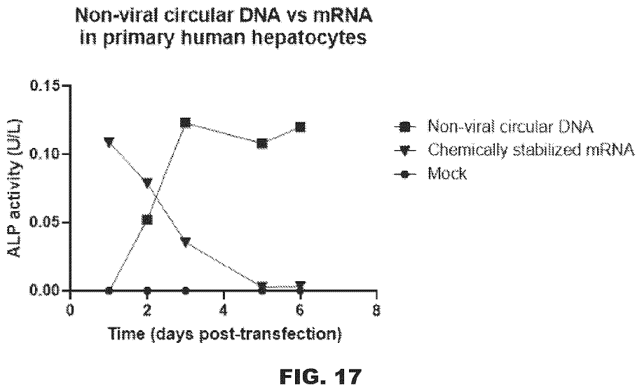

(i) A construct of the present disclosure including Cynomolgus Monkey Soluble TNALP DNA (SEQ ID NO: 43) with improved cruciform structure, and (ii) chemically modified mRNA encoding the same transgene were delivered to primary human hepatocytes using SM102-based lipid nanoparticle formulation. ALP activity from constructs according to the present disclosure in culture medium increased from day 1 to 3, then was maintained from day 3 to day 6. At the same time, ALP activity of mRNA-transfected cells kept dropping from day 1 to 5 and diminished at day 6 (FIG. 17).

### Method

Primary human hepatocytes (Lonza) were seeded on day 0 in duplicate wells of collagen type I-coated 96 well plates at 80,000 cells per well. One day post seeding, constructs of the present disclosure and control constructs were delivered by LNP mix.

Prepare SM-102-based lipid mix (SM102, DSPC, N/P 6, 1.5% DMG-PEG2K) and use pipette to mix DNA with lipid mix. Add lipid mix:DNA complexes dropwise to different areas of the wells. Gently rock the culture vessel back-and-forth and from side-to-side to evenly distribute lipid DNA mix.

Between day 1 and day 6, the cell culture medium was taken out and ALP level was determined by ALP fluorescence assay carried out on a Molecular Device plate reader iD5.

### Example 13—In Vitro Colocalization of Constructs According to the Present Disclosure with PARP1

FIG. 18 shows a representative confocal image of 293T cells transfected with a fluorescence-labelled construct according to the present disclosure (M012—SEQ ID NO: 28). Cells were stained with anti-PARP1-AF647 (Purple). As the area of PARP1 overlapped with the construct according to the present disclosure, it suggested that PARP1 might be involved in the intracellular pathway of constructs according to the present disclosure post-transfection.

### Method

A construct according to the present disclosure (M012—SEQ ID NO: 28) was labeled with green fluorescence DNA conjugation reagent before the transfection. Transfection Reagent was purchased from FuGENE and DNA transfection followed the manufacturer's recommendation.

293T cells were fixed on day 3 post-transfection, stained with DAPI and anti-PARP1 antibody, and mounted for confocal images. Purple color represents PARP1, DAPI staining (blue) represents the nucleus and green color represent the constructs according to the present disclosure inside the cell (FIG. 18).

### Example 14—Immunomodulatory Effects of Constructs According to the Present Disclosure

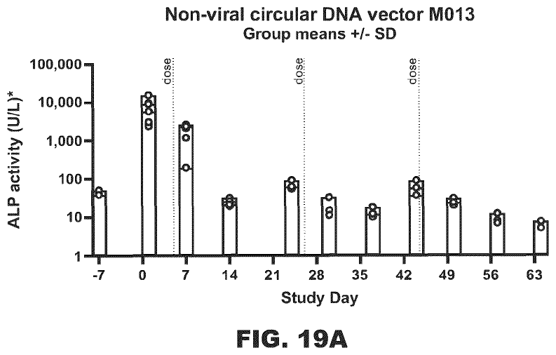

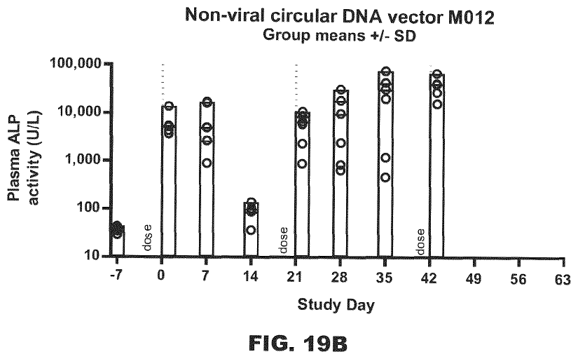

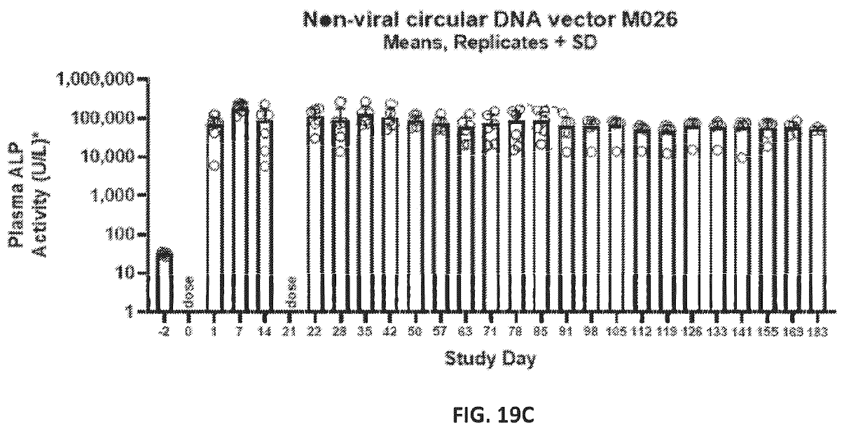

To determine the extent to which the cruciform structure and cassette gene impact immune response, adult mice (n=7 per group) were dosed three times with three different constructs: (i) a circular, non-viral DNA vector carrying the human TNALP with single inverted repeats without intervening heterologous sequences (M013—SEQ ID NO: 29) with high CpG content, bone-tag, and with lg-Fc domain (FIG. 19A); (ii) a circular, non-viral DNA vector according to the present disclosure carrying the human TNALP transgene with improved cruciform structure (M012—SEQ ID NO: 28) and high CpG content, bone-tag and Fc-domain (FIG. 19B), or (iii) a circular, non-viral DNA vector according to the present disclosure carrying cynomolgus monkey TNALP with improved cruciform structure (SEQ ID NO: 43), but with zero CpG content in transgene coding, no bone-tag, and no Ig-Fc-domain in the gene cassette to increase TNALP protein expression and reduce the immunostimulatory properties of the DNA (FIG. 19C). Plasma ALP activity from the DNA construct with single inverted repeats without intervening heterologous sequences cruciform structure was modest, non-durable and showed inability to re-dose likely due to the development of a humoral or cellular immune response against the inflammatory transgene product or transgene expressing cells. Plasma ALP activity from construct according to the present disclosure with improved cruciform structure showed increased potency, more durable transgene expression and ability to re-dose likely due to invasion of cytosolic DNA signaling (CDS) pathway mediated by the cGAS-STING pathway. Optimization of the gene expression cassette in that construct further improved plasma protein secretion and decreased immune response, leading to the highest observed levels of ALP activity.

### Method

Adult mice (strain: BALB/c) at age of 9-11 weeks were ordered from The Charles River Laboratories. Prior to study start, animals were randomized into groups using a random number generator. Non-viral DNA vector constructs were administered three times at 3-week interval to adult mice by hydrodynamic injection through the tail vein at dose of 15 μg per animal and volume of 100 mL/kg.

At different timepoints, plasma samples were collected and stored below −65° C. before the ALP assay was carried out. ALP activity was determined by TNALP fluorescence assays carried out on a Molecular Device plate reader iD5.

### ADDITIONAL EMBODIMENTS

In some embodiments, the present disclosure is directed to an amino acid sequence having at least 85% identity to any one of SEQ ID NOS: 19-27. In some embodiments, the present disclosure is directed to an amino acid sequence having at least 90% identity to any one of SEQ ID NOS: 19-27. In some embodiments, the present disclosure is directed to an amino acid sequence having at least 91% identity to any one of SEQ ID NOS: 19-27. In some embodiments, the present disclosure is directed to an amino acid sequence having at least 92% identity to any one of SEQ ID NOS: 19-27. In some embodiments, the present disclosure is directed to an amino acid sequence having at least 93% identity to any one of SEQ ID NOS: 19-27. In some embodiments, the present disclosure is directed to an amino acid sequence having at least 94% identity to any one of SEQ ID NOS: 19-27. In some embodiments, the present disclosure is directed to an amino acid sequence having at least 95% identity to any one of SEQ ID NOS: 19-27. In some embodiments, the present disclosure is directed to an amino acid sequence having at least 96% identity to any one of SEQ ID NOS: 19-27. In some embodiments, the present disclosure is directed to an amino acid sequence having at least 97% identity to any one of SEQ ID NOS: 19-27. In some embodiments, the present disclosure is directed to an amino acid sequence having at least 98% identity to any one of SEQ ID NOS: 19-27. In some embodiments, the present disclosure is directed to an amino acid sequence having at least 99% identity to any one of SEQ ID NOS: 19-27. In some embodiments, the present disclosure is directed to an amino acid sequence having any one of SEQ ID NOS: 19-27.

In some embodiments, the present disclosure is directed to a circular, non-viral DNA vector which includes a nucleus acid sequence encoding an amino acid having at least 85% identity to any one of SEQ ID NOS: 19-27. In some embodiments, the present disclosure is directed to a circular, non-viral DNA vector which includes a nucleus acid sequence encoding encoding an amino acid having at least 90% identity to any one of SEQ ID NOS: 19-27. In some embodiments, the present disclosure is directed to a circular, non-viral DNA vector which includes a nucleus acid sequence encoding encoding an amino acid having at least 91% identity to any one of SEQ ID NOS: 19-27. In some embodiments, the present disclosure is directed to a circular, non-viral DNA vector which includes a nucleus acid sequence encoding encoding an amino acid having at least 92% identity to any one of SEQ ID NOS: 19-27. In some embodiments, the present disclosure is directed to a circular, non-viral DNA vector which includes a nucleus acid sequence encoding encoding an amino acid having at least 93% identity to any one of SEQ ID NOS: 19-27. In some embodiments, the present disclosure is directed to a circular, non-viral DNA vector which includes a nucleus acid sequence encoding encoding an amino acid having at least 94% identity to any one of SEQ ID NOS: 19-27. In some embodiments, the present disclosure is directed to a circular, non-viral DNA vector which includes a nucleus acid sequence encoding encoding an amino acid having at least 95% identity to any one of SEQ ID NOS: 19-27. In some embodiments, the present disclosure is directed to a circular, non-viral DNA vector which includes a nucleus acid sequence encoding encoding an amino acid having at least 96% identity to any one of SEQ ID NOS: 19-27. In some embodiments, the present disclosure is directed to a circular, non-viral DNA vector which includes a nucleus acid sequence encoding encoding an amino acid having at least 97% identity to any one of SEQ ID NOS: 19-27. In some embodiments, the present disclosure is directed to a circular, non-viral DNA vector which includes a nucleus acid sequence encoding encoding an amino acid having at least 98% identity to any one of SEQ ID NOS: 19-27. In some embodiments, the present disclosure is directed to a circular, non-viral DNA vector which includes a nucleus acid sequence encoding encoding an amino acid having at least 99% identity to any one of SEQ ID NOS: 19-27. In some embodiments, the present disclosure is directed to a circular, non-viral DNA vector which includes a nucleus acid sequence encoding encoding an amino acid having any one of SEQ ID NOS: 19-27.

In some embodiments, the present disclosure is directed to a nucleic acid sequence having at least 80% identity to any one of SEQ ID NOS: 28-73. In some embodiments, the present disclosure is directed to a nucleic acid sequence having at least 85% identity to any one of SEQ ID NOS: 28-73. In some embodiments, the present disclosure is directed to a nucleic acid sequence having at least 86% identity to any one of SEQ ID NOS: 28-73. In some embodiments, the present disclosure is directed to a nucleic acid sequence having at least 87% identity to any one of SEQ ID NOS: 28-73. In some embodiments, the present disclosure is directed to a nucleic acid sequence having at least 88% identity to any one of SEQ ID NOS: 28-73. In some embodiments, the present disclosure is directed to a nucleic acid sequence having at least 89% identity to any one of SEQ ID NOS: 28-73. In some embodiments, the present disclosure is directed to a nucleic acid sequence having at least 90% identity to any one of SEQ ID NOS: 28-73. In some embodiments, the present disclosure is directed to a nucleic acid sequence having at least 91% identity to any one of SEQ ID NOS: 28-73. In some embodiments, the present disclosure is directed to a nucleic acid sequence having at least 92% identity to any one of SEQ ID NOS: 28-73. In some embodiments, the present disclosure is directed to a nucleic acid sequence having at least 93% identity to any one of SEQ ID NOS: 28-73. In some embodiments, the present disclosure is directed to a nucleic acid sequence having at least 94% identity to any one of SEQ ID NOS: 28-73. In some embodiments, the present disclosure is directed to a nucleic acid sequence having at least 95% identity to any one of SEQ ID NOS: 28-73. In some embodiments, the present disclosure is directed to a nucleic acid sequence having at least 96% identity to any one of SEQ ID NOS: 28-73. In some embodiments, the present disclosure is directed to a nucleic acid sequence having at least 97% identity to any one of SEQ ID NOS: 28-73. In some embodiments, the present disclosure is directed to a nucleic acid sequence having at least 98% identity to any one of SEQ ID NOS: 28-73. In some embodiments, the present disclosure is directed to a nucleic acid sequence having at least 99% identity to any one of SEQ ID NOS: 28-73. In some embodiments, the present disclosure is directed to a nucleic acid sequence having at least any one of SEQ ID NOS: 28-73.

A circular, non-viral DNA vector comprising a nucleic acid sequence having at least 80% identity to any one of SEQ ID NOS: 28-30. A circular, non-viral DNA vector comprising a nucleic acid sequence having at least 81% identity to any one of SEQ ID NOS: 28-30. A circular, non-viral DNA vector comprising a nucleic acid sequence having at least 80% identity to any one of SEQ ID NOS: 28-30. A circular, non-viral DNA vector comprising a nucleic acid sequence having at least 81% identity to any one of SEQ ID NOS: 28-30. A circular, non-viral DNA vector comprising a nucleic acid sequence having at least 82% identity to any one of SEQ ID NOS: 28-30. A circular, non-viral DNA vector comprising a nucleic acid sequence having at least 83% identity to any one of SEQ ID NOS: 28-30. A circular, non-viral DNA vector comprising a nucleic acid sequence having at least 84% identity to any one of SEQ ID NOS: 28-30. A circular, non-viral DNA vector comprising a nucleic acid sequence having at least 85% identity to any one of SEQ ID NOS: 28-30. A circular, non-viral DNA vector comprising a nucleic acid sequence having at least 86% identity to any one of SEQ ID NOS: 28-30. A circular, non-viral DNA vector comprising a nucleic acid sequence having at least 87% identity to any one of SEQ ID NOS: 28-30. A circular, non-viral DNA vector comprising a nucleic acid sequence having at least 88% identity to any one of SEQ ID NOS: 28-30. A circular, non-viral DNA vector comprising a nucleic acid sequence having at least 89% identity to any one of SEQ ID NOS: 28-30. A circular, non-viral DNA vector comprising a nucleic acid sequence having at least 90% identity to any one of SEQ ID NOS: 28-30. A circular, non-viral DNA vector comprising a nucleic acid sequence having at least 91% identity to any one of SEQ ID NOS: 28-30. A circular, non-viral DNA vector comprising a nucleic acid sequence having at least 92% identity to any one of SEQ ID NOS: 28-30. A circular, non-viral DNA vector comprising a nucleic acid sequence having at least 93% identity to any one of SEQ ID NOS: 28-30. A circular, non-viral DNA vector comprising a nucleic acid sequence having at least 94% identity to any one of SEQ ID NOS: 28-30. A circular, non-viral DNA vector comprising a nucleic acid sequence having at least 95% identity to any one of SEQ ID NOS: 28-30. A circular, non-viral DNA vector comprising a nucleic acid sequence having at least 96% identity to any one of SEQ ID NOS: 28-30. A circular, non-viral DNA vector comprising a nucleic acid sequence having at least 97% identity to any one of SEQ ID NOS: 28-30. A circular, non-viral DNA vector comprising a nucleic acid sequence having at least 98% identity to any one of SEQ ID NOS: 28-30. A circular, non-viral DNA vector comprising a nucleic acid sequence having at least 99% identity to any one of SEQ ID NOS: 28-30. A circular, non-viral DNA vector comprising a nucleic acid sequence having any one of SEQ ID NOS: 28-30.

A circular, non-viral DNA vector comprising a nucleic acid sequence having at least 80% identity to any one of SEQ 1D NOS: 72-73. A circular, non-viral DNA vector comprising a nucleic acid sequence having at least 81% identity to any one of SEQ ID NOS: 72-73. A circular, non-viral DNA vector comprising a nucleic acid sequence having at least 80% identity to any one of SEQ ID NOS: 72-73. A circular, non-viral DNA vector comprising a nucleic acid sequence having at least 81% identity to any one of SEQ ID NOS: 72-73. A circular, non-viral DNA vector comprising a nucleic acid sequence having at least 82% identity to any one of SEQ ID NOS: 72-73. A circular, non-viral DNA vector comprising a nucleic acid sequence having at least 83% identity to any one of SEQ ID NOS: 72-73. A circular, non-viral DNA vector comprising a nucleic acid sequence having at least 84% identity to any one of SEQ ID NOS: 72-73. A circular, non-viral DNA vector comprising a nucleic acid sequence having at least 85% identity to any one of SEQ ID NOS: 72-73. A circular, non-viral DNA vector comprising a nucleic acid sequence having at least 86% identity to any one of SEQ ID NOS: 72-73. A circular, non-viral DNA vector comprising a nucleic acid sequence having at least 87% identity to any one of SEQ ID NOS: 72-73. A circular, non-viral DNA vector comprising a nucleic acid sequence having at least 88% identity to any one of SEQ ID NOS: 72-73. A circular, non-viral DNA vector comprising a nucleic acid sequence having at least 89% identity to any one of SEQ ID NOS: 72-73. A circular, non-viral DNA vector comprising a nucleic acid sequence having at least 90% identity to any one of SEQ ID NOS: 72-73. A circular, non-viral DNA vector comprising a nucleic acid sequence having at least 91% identity to any one of SEQ ID NOS: 72-73. A circular, non-viral DNA vector comprising a nucleic acid sequence having at least 92% identity to any one of SEQ ID NOS: 72-73. A circular, non-viral DNA vector comprising a nucleic acid sequence having at least 93% identity to any one of SEQ ID NOS: 72-73. A circular, non-viral DNA vector comprising a nucleic acid sequence having at least 94% identity to any one of SEQ ID NOS: 72-73. A circular, non-viral DNA vector comprising a nucleic acid sequence having at least 95% identity to any one of SEQ ID NOS: 72-73. A circular, non-viral DNA vector comprising a nucleic acid sequence having at least 96% identity to any one of SEQ ID NOS: 72-73. A circular, non-viral DNA vector comprising a nucleic acid sequence having at least 97% identity to any one of SEQ ID NOS: 72-73. A circular, non-viral DNA vector comprising a nucleic acid sequence having at least 98% identity to any one of SEQ ID NOS: 72-73. A circular, non-viral DNA vector comprising a nucleic acid sequence having at least 99% identity to any one of SEQ ID NOS: 72-73. A circular, non-viral DNA vector comprising a nucleic acid sequence having any one of SEQ lD NOS: 72-73.

In some embodiments, is an isolated, circular, non-viral DNA vector comprises: a first portion consisting essentially of an expression cassette including one or more nucleic acid sequences encoding one or more therapeutic proteins, where each of the one or more nucleic acid sequences encoding the one or more therapeutic proteins are operatively linked to a promoter; and a second portion capable of forming at least one cruciform structure, wherein the second portion consists essentially of at least two inverted repeat sequences, and where the at least two inverted repeat sequences are separated by a non-repeating nucleotide sequence having at least 3 nucleotides.

In some embodiments, is an isolated, circular, non-viral DNA vector consisting essentially of: a first portion consisting essentially of an expression cassette including one or more nucleic acid sequences encoding one or more therapeutic proteins, where each of the one or more nucleic acid sequences encoding the one or more therapeutic proteins are operatively linked to a promoter; and a second portion capable of forming at least one cruciform structure, wherein the second portion consists essentially of at least two inverted repeat sequences, and where the at least two inverted repeat sequences are separated by a non-repeating nucleotide sequence having at least 3 nucleotides.

In some embodiments, is an isolated, circular, non-viral DNA vector comprises: a first portion consisting of an expression cassette including one or more nucleic acid sequences encoding one or more therapeutic proteins, where each of the one or more nucleic acid sequences encoding the one or more therapeutic proteins are operatively linked to a promoter; and a second portion capable of forming at least one cruciform structure, wherein the second portion consists of at least two inverted repeat sequences, and where the at least two inverted repeat sequences are separated by a non-repeating nucleotide sequence having at least 3 nucleotides.

In some embodiments, is an isolated, circular, non-viral DNA vector consists of: a first portion consisting of an expression cassette including one or more nucleic acid sequences encoding one or more therapeutic proteins, where each of the one or more nucleic acid sequences encoding the one or more therapeutic proteins are operatively linked to a promoter; and a second portion capable of forming at least one cruciform structure, wherein the second portion consists of at least two inverted repeat sequences, and where the at least two inverted repeat sequences are separated by a non-repeating nucleotide sequence having at least 3 nucleotides.

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure.

What is claimed is:

1. An isolated, circular, non-viral, non-integrating DNA vector comprising:
(i) a first portion comprising an expression cassette comprising a nucleic acid sequence encoding a therapeutic protein, wherein the nucleic acid sequence encoding the therapeutic protein is operably linked to a promoter; and
(ii) a second portion comprising a nucleic acid sequence consisting essentially of a first adeno-associated virus (AAV) inverted repeat sequence, an R6Kγ bacterial origin of replication (oriR6Kγ) sequence, and a second AAV inverted repeat sequence;
wherein the first AAV inverted repeat sequence, R6Kγori sequence, and second AAV inverted repeat sequence are contiguous, and wherein the R6Kγori sequence is flanked by, and in between, the first AAV inverted repeat sequence and the second AAV inverted repeat sequence.

2. The DNA vector of claim 1, wherein the oriR6Kγ sequence comprises a nucleic acid sequence selected from SEQ ID NO: 58 or SEQ ID NO: 59, or a nucleic acid sequence at least 90% identical thereto.

3. The DNA vector of claim 2, wherein the oriR6Kγ sequence comprises a nucleic acid sequence selected from SEQ ID NO: 58 or SEQ ID NO: 59.

4. The DNA vector of claim 1, wherein each of the AAV inverted repeat sequences is independently at least 90% identical to a sequence selected from the group consisting of SEQ ID NOS: 1-18.

5. The DNA vector of claim 4, wherein each of the AAV inverted repeat sequences is independently selected from the group consisting of SEQ ID NOS: 1-18.

6. The DNA vector of claim 1, wherein the first AAV inverted repeat sequence is at least 90% identical to a sequence selected from the group consisting of SEQ ID NOS: 1, 3, 5, 7, 9, 11, 13, 15, and 17; wherein the ori6Kγ sequence comprises a nucleic acid sequence at least 90% identical to a sequence selected from the group consisting of SEQ ID NO: 58 and SEQ ID NO: 59; and wherein the second AAV inverted repeat is at least 90% identical to a sequence selected from the group consisting of SEQ ID NOS: 2, 4, 6, 8, 10, 12, 14, 16, and 18.

7. The DNA vector of claim 6, wherein the first AAV inverted repeat sequence is selected from the group consisting of SEQ ID NOS: 1, 3, 5, 7, 9, 11, 13, 15, and 17; wherein the ori6Kγ sequence comprises a nucleic acid sequence selected from the group consisting of SEQ ID NO: 58 and SEQ ID NO: 59; and wherein the second AAV inverted repeat sequence is selected from the group consisting of SEQ ID NOS: 2, 4, 6, 8, 10, 12, 14, 16, and 18.

8. The DNA vector of claim 1, wherein the second portion comprises a sequence selected from the group consisting of SEQ ID NOS: 35, 60, 62, and 71, or a sequence at least about 95% identical thereto.

9. The DNA vector of claim 8, wherein the second portion comprises a sequence selected from the group consisting of SEQ ID NOS: 35, 60, 62, and 71.

10. An isolated, circular, non-viral, non-integrating DNA vector comprising:
(i) a first portion comprising an expression cassette comprising a nucleic acid sequence encoding a therapeutic protein, wherein the nucleic acid sequence encoding the therapeutic protein is operably linked to a promoter; and
(ii) a second portion comprising a nucleic acid sequence comprising the Formula X—Y—X′;
wherein X and X′ are each separately an AAV inverted repeat sequence and Y consists essentially of oriR6Kγ sequence.

11. The DNA vector of claim 10, wherein the oriR6Kγ comprises a nucleic acid sequence selected from SEQ ID NO: 58 or SEQ ID NO: 59, or a nucleic acid sequence at least 90% identical thereto.

12. The DNA vector of claim 11, wherein the oriR6Kγ comprises a nucleic acid sequence selected from SEQ ID NO: 58 or SEQ ID NO: 59.

13. The DNA vector of claim 10, wherein X and X′ are independently at least 90% identical to a sequence selected from the group consisting of SEQ ID NOS: 1-18.

14. The DNA vector of claim 13, wherein X and X′ are independently selected from the group consisting of SEQ ID NOS: 1-18.

15. The DNA vector of claim 10, wherein X is at least 90% identical to a sequence selected from the group consisting of SEQ ID NOS: 1, 3, 5, 7, 9, 11, 13, 15, and 17; wherein the ori6Kγ sequence comprises a nucleic acid sequence at least 90% identical to a sequence selected from the group consisting of SEQ ID NO: 58 and SEQ ID NO: 59; and wherein Y is at least 90% identical to a sequence selected from the group consisting of SEQ ID NOS: 2, 4, 6, 8, 10, 12, 14, 16, and 18.

16. The DNA vector of claim 15, wherein X is selected from the group consisting of SEQ ID NOS: 1, 3, 5, 7, 9, 11, 13, 15, and 17; wherein the ori6Kγ sequence comprises a nucleic acid sequence selected from the group consisting of SEQ ID NO: 58 and SEQ ID NO: 59; and wherein X′ is selected from the group consisting of SEQ ID NOS: 2, 4, 6, 8, 10, 12, 14, 16, and 18.

17. The DNA vector of claim 10, wherein the second portion comprises a sequence at least 95% identical to a sequence selected from the group consisting of SEQ ID NOS: 35, 60, 62, and 71, or a sequence at least about 95% identical thereto.

18. The DNA vector of claim 17, wherein the second portion comprises a sequence selected from the group consisting of SEQ ID NOS: 35, 60, 62, and 71.

19. An isolated, circular, non-viral, non-integrating DNA vector comprising:
(i) a first portion comprising an expression cassette comprising a nucleic acid sequence encoding a therapeutic protein, wherein the nucleic acid sequence encoding the therapeutic protein is operably linked to a promoter; and
(ii) a second portion comprising a nucleic acid sequence consisting essentially of, operably linked in a 5′ to a 3′ direction:
(a) a first AAV inverted repeat sequence;
(b) an oriR6Kγ sequence; and
(c) a second AAV inverted repeat sequence.

20. The DNA vector of claim 19, wherein the oriR6Kγ sequence comprises a nucleic acid sequence selected from SEQ ID NO: 58 or SEQ ID NO: 59, or a nucleic acid sequence at least 90% identical thereto.

21. The DNA vector of claim 20, wherein the oriR6Kγ sequence comprises a nucleic acid sequence selected from SEQ ID NO: 58 or SEQ ID NO: 59.

22. The DNA vector of claim 19, wherein each of the AAV inverted repeat sequences is independently at least 90% identical to a sequence selected from the group consisting of SEQ ID NOS: 1-18.

23. The DNA vector of claim 22, wherein each of the AAV inverted repeat sequences is independently selected from the group consisting of SEQ ID NOS: 1-18.

24. The DNA vector of claim 19, wherein the first AAV inverted repeat sequence is at least 90% identical to a sequence selected from the group consisting of SEQ ID NOS: 1, 3, 5, 7, 9, 11, 13, 15, and 17; wherein the ori6Kγ sequence comprises a nucleic acid sequence at least 90% identical to a sequence selected from the group consisting of SEQ ID NO: 58 and SEQ ID NO: 59; and wherein the second AAV inverted repeat is at least 90% identical to a sequence selected from the group consisting of SEQ ID NOS: 2, 4, 6, 8, 10, 12, 14, 16, and 18.

25. The DNA vector of claim 24, wherein the first AAV inverted repeat sequence is selected from the group consisting of SEQ ID NOS: 1, 3, 5, 7, 9, 11, 13, 15, and 17; wherein the ori6Kγ sequence comprises a nucleic acid sequence selected from the group consisting of SEQ ID NO: 58 and SEQ ID NO: 59; and wherein the second AAV inverted repeat sequence is selected from the group consisting of SEQ ID NOS: 2, 4, 6, 8, 10, 12, 14, 16, and 18.

26. The DNA vector of claim 19, wherein the second portion comprises a sequence selected from the group consisting of SEQ ID NOS: 35, 60, 62, and 71, or a sequence at least about 95% identical thereto.

27. The DNA vector of claim 26, wherein the second portion comprises a sequence selected from the group consisting of SEQ ID NOS: 35, 60, 62, and 71.


SEQUENCE LISTING (83 sequences)

SEQ ID NO: 1 - DNA, 130 bp, synthetic construct
  misc_feature 1..130: note = Synthetic: 5' Inverted Repeat Sequence derived from AAV2 ITR
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 2 - DNA, 130 bp, synthetic construct
  misc_feature 1..130: note = Synthetic: 3' Inverted Repeat Sequence derived from AAV2 ITR
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 3 - DNA, 128 bp, synthetic construct
  misc_feature 1..128: note = Synthetic: 5' Inverted Repeat Sequence derived from AAV1 ITR
  source 1..128: mol_type = other DNA
ttacccctagtgatggagttgcccactccctctctgcgcgctcgctcgctcggtggggcctgcggaccaaaggtccgcagacggcagagctctgctctgccggccccaccgagcgagcgagcgcgcag

SEQ ID NO: 4 - DNA, 128 bp, synthetic construct
  misc_feature 1..128: note = Synthetic: 3' Inverted Repeat Sequence derived from AAV1 ITR
  source 1..128: mol_type = other DNA
ctgcgcgctcgctcgctcggtggggccggcagagcagagctctgccgtctgcggacctttggtccgcaggccccaccgagcgagcgagcgcgcagagagggagtgggcaactccatcactaggggtaa

SEQ ID NO: 5 - DNA, 133 bp, synthetic construct
  misc_feature 1..133: note = Synthetic: 5' Inverted Repeat Sequence derived from AAV3 ITR
  source 1..133: mol_type = other DNA
gccatacctctagtgatggagttgcccactccctctatgcgcactcgctcgctcggtggggcctggcgaccaaaggtcgccagacggacgtgctttgcacgtccggccccaccgagcgagcgagtgcgcatag

SEQ ID NO: 6 - DNA, 133 bp, synthetic construct
  misc_feature 1..133: note = Synthetic: 3' Inverted Repeat Sequence derived from AAV3 ITR
  source 1..133: mol_type = other DNA
ctatgcgcactcgctcgctcggtggggccggacgtgcaaagcacgtccgtctggcgacctttggtcgccaggccccaccgagcgagcgagtgcgcatagagggagtgggcaactccatcactagaggtatggc

SEQ ID NO: 7 - DNA, 133 bp, synthetic construct
  misc_feature 1..133: note = Synthetic: 5' Inverted Repeat Sequence derived from AAV4 ITR
  source 1..133: mol_type = other DNA
gggcaaacctagatgatggagttggccactccctctatgcgcgctcgctcactcactcggccctggagaccaaaggtctccagactgccggcctctggccggcagggccgagtgagtgagcgagcgcgcatag

SEQ ID NO: 8 - DNA, 133 bp, synthetic construct
  misc_feature 1..133: note = Synthetic: 3' Inverted Repeat Sequence derived from AAV4 ITR
  source 1..133: mol_type = other DNA
ctatgcgcgctcgctcactcactcggccctgccggccagaggccggcagtctggagacctttggtctccagggccgagtgagtgagcgagcgcgcatagagggagtggccaactccatcatctaggtttgccc

SEQ ID NO: 9 - DNA, 129 bp, synthetic construct
  misc_feature 1..129: note = Synthetic: 5' Inverted Repeat Sequence derived from AAV6 ITR
  source 1..129: mol_type = other DNA
atacccctagtgatggagttgcccactccctctatgcgcgctcgctcgctcgtggggcctgcggaccaaaggtccgcagacggcagagctctgctctgccggccccaccgagcgagcgagcgcgcatag

SEQ ID NO: 10 - DNA, 129 bp, synthetic construct
  misc_feature 1..129: note = Synthetic: 3' Inverted Repeat Sequence derived from AAV6 ITR
  source 1..129: mol_type = other DNA
ctatgcgcgctcgctcgctcggtggggccggcagagcagagctctgccgtctgcggacctttggtccgcaggccccacgagcgagcgagcgcgcatagagggagtgggcaactccatcactaggggtat

SEQ ID NO: 11 - DNA, 132 bp, synthetic construct
  misc_feature 1..132: note = Synthetic: 5' Inverted Repeat Sequence derived from AAV7 ITR
  source 1..132: mol_type = other DNA
cggtacccctagtgagggagttggccactccctctatgcgcgctcgctcgctcggtggggcctgcggaccaaaggtccgcagacggcagagctctgctctgccggccccaccgagcgagcgagcgcgcatag

SEQ ID NO: 12 - DNA, 132 bp, synthetic construct
  misc_feature 1..132: note = Synthetic: 3' Inverted Repeat Sequence derived from AAV7 ITR
  source 1..132: mol_type = other DNA
ctatgcgcgctcgctcgctcggtggggccggcagagcagagctctgccgtctgcggacctttggtccgcaggccccaccgagcgagcgagcgcgcatagagggagtggccaactccctcactaggggtaccg

SEQ ID NO: 13 - DNA, 129 bp, synthetic construct
  misc_feature 1..129: note = Synthetic: Zero-CpG 5' Inverted Repeat Sequence Variant 1 derived from AAV2 ITR
  source 1..129: mol_type = other DNA
ttaattaaggctaggaacccctagtgatggagttggccactccctctctgccctctccctctctcactgaggcctggggaccaaaggtccccagactgcagagctttgctctgcaggcctcagtgagag

SEQ ID NO: 14 - DNA, 129 bp, synthetic construct
  misc_feature 1..129: note = Synthetic: Zero-CpG 3' Inverted Repeat Sequence Variant 1 derived from AAV2 ITR
  source 1..129: mol_type = other DNA
ctctcactgaggcctgcagagcaaagctctgcagtctggggacctttggtccccaggcctcagtgagagagggagagggcagagagggagtggccaactccatcactaggggttcctagccttaattaa

SEQ ID NO: 15 - DNA, 132 bp, synthetic construct
  misc_feature 1..132: note = Synthetic: Zero-CpG 5' Inverted Repeat Sequence Variant 2 derived from AAV2 ITR
  source 1..132: mol_type = other DNA
aggaacccctagtgatggagttggccactccctctctgcactctctctctctcactgaggcctggggaccaaaggtccccagactgcagagctttgctctgcaggcctcagtgagagagagagagtgcagag

SEQ ID NO: 16 - DNA, 129 bp, synthetic construct; identical to SEQ ID NO: 14
  misc_feature 1..129: note = Synthetic: Zero-CpG 3' Inverted Repeat Sequence Variant 2 derived from AAV2 ITR
  source 1..129: mol_type = other DNA

SEQ ID NO: 17 - DNA, 129 bp, synthetic construct; identical to SEQ ID NO: 13
  misc_feature 1..129: note = Synthetic: Zero-CpG 5' Inverted Repeat Sequence Variant 3 derived from AAV2 ITR
  source 1..129: mol_type = other DNA

SEQ ID NO: 18 - DNA, 129 bp, synthetic construct; identical to SEQ ID NO: 14
  misc_feature 1..129: note = Synthetic: Zero-CpG 3' Inverted Repeat Sequence Variant 3 derived from AAV2 ITR
  source 1..129: mol_type = other DNA

SEQ ID NO: 19 - protein, 502 aa, synthetic construct
  REGION 1..502: note = Synthetic: sTNALP
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASS

SEQ ID NO: 20 - protein, 512 aa, synthetic construct
  REGION 1..512: note = Synthetic: TNALP-D10
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSDDDDDDDDDD

SEQ ID NO: 21 - protein, 520 aa, synthetic construct
  REGION 1..520: note = Synthetic: sTNALP-(DSS)6
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSDSSDSSDSSDSSDSSDSS

SEQ ID NO: 22 - protein, 231 aa, synthetic construct
  REGION 1..231: note = Synthetic: Fc Domain
LKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDI

SEQ ID NO: 23 - protein, 524 aa, Homo sapiens
  REGION 1..524: note = misc_feature - HUMAN Alkaline phosphatase, tissue-nonspecific isozyme
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF

SEQ ID NO: 24 - protein, 484 aa, synthetic construct
  REGION 1..484: note = Synthetic: secretive TNALP without GPI
VPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASS

SEQ ID NO: 25 - protein, 18 aa, synthetic construct
  REGION 1..18: note = Synthetic: signal peptide
MISPFLVLAIGTCLTNSL

SEQ ID NO: 26 - protein, 22 aa, synthetic construct
  REGION 1..22: note = Synthetic: GPI anchor
AGSLAAGPLLLALALYPLSVLF

SEQ ID NO: 27 - protein, 22 aa, synthetic construct
  REGION 1..22: note = Synthetic: mutated GPI anchor
AGSLAADPAATAAALYPLSVLF

SEQ ID NO: 28 - DNA, 5118 bp, synthetic construct
  misc_feature 1..5118: note = Synthetic: (M012) Halo sTNALP vector comprising a cruciform structure IR-R6K Ori-IR and EF1A-driven sTNALP-Fc-(DSS)6 transgene
  source 1..5118: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcgccaccatggatatgcgcgtacccgctcaacttctgggtttgttgctgctttggctcaggggagcccgctgtgccgcgcttgtccctgaaaaggagaaggaccccaaatattggagagaccaggctcaggaaaccctcaagtacgctcttgaactgcaaaagcttaatactaacgtggctaaaaacgtgataatgttcttgggggacggaatgggggtttcaaccgttaccgcggccagaatcttgaaagggcagctgcaccataacccaggcgaagaaacgaggctggagatggacaaatttccattcgtggcgctttctaagacctacaacactaacgcccaggtccctgactcagctggtacggccactgcctacctttgtggagtgaaggcaaatgaggggactgttggtgtttccgcagctactgagagatctcggtgtaatacgactcaaggtaatgaagttacatccatactccgatgggcaaaagacgcgggcaaatctgttgggattgttacaactactagggtgaaccatgcgacaccctctgcagcttatgcgcacagtgccgaccgagactggtattcagataatgagatgccacccgaggcccttagccagggctgcaaggacatagcctatcaattgatgcacaatatcagagacatagacgttataatgggtggtggtcgcaagtacatgtaccccaaaaacaaaaccgacgtagagtatgaatccgatgaaaaagctaggggcaccagactcgacggacttgatctggttgatacatggaagagcttcaagcccagatacaagcattcacatttcatctggaacaggaccgaacttctcacattggacccccacaatgttgactatctcctcggtttgtttgaaccaggggatatgcagtatgagctcaatcggaacaacgtcactgaccccagtttgagtgaaatggtcgttgttgcaatacaaattttgaggaaaaatccaaaggggtttttcttgttggtagagggcggacggatagatcacgggcaccacgaaggcaaagcaaaacaggccctgcacgaagccgttgaaatggatcgcgctatcggtcaagccggctccctgacgagtagtgaagataccttgaccgtcgtcactgcggatcatagccacgtttttacctttgggggatacacccccaggggcaactcaatttttggacttgcaccgatgctcagcgacacagataaaaagccttttactgcgattctgtatggcaacggccctggatacaaagtagtagggggtgaaagggaaaacgtcagcatggtggactatgcacacaataattatcaggcgcagtccgctgttccgctgcggcatgagactcacgggggagaggacgttgctgtcttttccaagggaccaatggctcatcttctccatggagtccatgagcaaaattatgtccctcatgtcatggcatacgctgcatgtataggagcaaatctcgggcactgtgcaccggcgtcctccctcaaagacaaaacacacacttgtccaccttgcccggcacctgaacttctgggcggcccgagcgtttttttgttccctcctaaacctaaggacacgctcatgatcagccgcacgccagaggttacctgtgtagtggtggatgtcagccacgaggatccggaagtaaaattcaactggtatgtagatggggtggaggttcacaatgccaaaaccaagccacgagaggaacagtacaatagtacttaccgggtcgtgtctgtattgaccgtcctgcatcaggattggttgaacgggaaagaatataagtgcaaggtgagcaacaaagcacttccggccccaatagagaaaacaattagtaaagcaaaaggacaaccacgggaaccgcaagtttatacactgccgccctctcgagacgagctgaccaaaaatcaggttagccttacatgtttggtcaaggggttctacccgagtgatattgcagtggaatgggaatccaacggtcaaccagaaaataactacaaaactactccgccggtgttggattcagacggctctttctttctgtatagcaaactcactgttgacaagtcaagatggcagcagggcaatgtatttagctgtagtgttatgcacgaagctcttcataaccactacacacaaaagtcactttctctttccccaggaaaagacatcgattccagcgacagttcagacagctctgacagctccgattcctccgactcaagttgaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaataccgttcgtataatgtatgctatacgaacggtactgcagtgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcttaattaagg

SEQ ID NO: 29 - DNA, 4857 bp, synthetic construct
  misc_feature 1..4857: note = Synthetic: (M013) Halo sTNALP vector comprising A CRUCIFORM structure and EF1A-driven sTNALP-Fc-(DSS)6 transgene
  source 1..4857: mol_type = other DNA
ctaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggtttcctttaattaaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcgccaccatggatatgcgcgtacccgctcaacttctgggtttgttgctgctttggctcaggggagcccgctgtgccgcgcttgtccctgaaaaggagaaggaccccaaatattggagagaccaggctcaggaaaccctcaagtacgctcttgaactgcaaaagcttaatactaacgtggctaaaaacgtgataatgttcttgggggacggaatgggggtttcaaccgttaccgcggccagaatcttgaaagggcagctgcaccataacccaggcgaagaaacgaggctggagatggacaaatttccattcgtggcgctttctaagacctacaacactaacgcccaggtccctgactcagctggtacggccactgcctacctttgtggagtgaaggcaaatgaggggactgttggtgtttccgcagctactgagagatctcggtgtaatacgactcaaggtaatgaagttacatccatactccgatgggcaaaagacgcgggcaaatctgttgggattgttacaactactagggtgaaccatgcgacaccctctgcagcttatgcgcacagtgccgaccgagactggtattcagataatgagatgccacccgaggcccttagccagggctgcaaggacatagcctatcaattgatgcacaatatcagagacatagacgttataatgggtggtggtcgcaagtacatgtaccccaaaaacaaaaccgacgtagagtatgaatccgatgaaaaagctaggggcaccagactcgacggacttgatctggttgatacatggaagagcttcaagcccagatacaagcattcacatttcatctggaacaggaccgaacttctcacattggacccccacaatgttgactatctcctcggtttgtttgaaccaggggatatgcagtatgagctcaatcggaacaacgtcactgaccccagtttgagtgaaatggtcgttgttgcaatacaaattttgaggaaaaatccaaaggggtttttcttgttggtagagggcggacggatagatcacgggcaccacgaaggcaaagcaaaacaggccctgcacgaagccgttgaaatggatcgcgctatcggtcaagccggctccctgacgagtagtgaagataccttgaccgtcgtcactgcggatcatagccacgtttttacctttgggggatacacccccaggggcaactcaatttttggacttgcaccgatgctcagcgacacagataaaaagccttttactgcgattctgtatggcaacggccctggatacaaagtagtagggggtgaaagggaaaacgtcagcatggtggactatgcacacaataattatcaggcgcagtccgctgttccgctgcggcatgagactcacgggggagaggacgttgctgtcttttccaagggaccaatggctcatcttctccatggagtccatgagcaaaattatgtccctcatgtcatggcatacgctgcatgtataggagcaaatctcgggcactgtgcaccggcgtcctccctcaaagacaaaacacacacttgtccaccttgcccggcacctgaacttctgggcggcccgagcgtttttttgttccctcctaaacctaaggacacgctcatgatcagccgcacgccagaggttacctgtgtagtggtggatgtcagccacgaggatccggaagtaaaattcaactggtatgtagatggggtggaggttcacaatgccaaaaccaagccacgagaggaacagtacaatagtacttaccgggtcgtgtctgtattgaccgtcctgcatcaggattggttgaacgggaaagaatataagtgcaaggtgagcaacaaagcacttccggccccaatagagaaaacaattagtaaagcaaaaggacaaccacgggaaccgcaagtttatacactgccgccctctcgagacgagctgaccaaaaatcaggttagccttacatgtttggtcaaggggttctacccgagtgatattgcagtggaatgggaatccaacggtcaaccagaaaataactacaaaactactccgccggtgttggattcagacggctctttctttctgtatagcaaactcactgttgacaagtcaagatggcagcagggcaatgtatttagctgtagtgttatgcacgaagctcttcataaccactacacacaaaagtcactttctctttccccaggaaaagacatcgattccagcgacagttcagacagctctgacagctccgattcctccgactcaagttgaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggataccgttcgtataatgtatgctatacgaacggtactgcagtgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcttaattaagg

SEQ ID NO: 30 - DNA, 4233 bp, synthetic construct
  misc_feature 1..4233: note = Synthetic: (M014) Halo firefly luciferase vector comprising a cruciform structure and EF1A-driven firefly luciferase reporter transgene
  source 1..4233: mol_type = other DNA
ctaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggtttcctttaattaaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcgccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtgaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggataccgttcgtataatgtatgctatacgaacggtactgcagtgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcttaattaagg

SEQ ID NO: 31 - DNA, 5708 bp, synthetic construct
  misc_feature 1..5708: note = Synthetic: P021 EF1a-driven firefly luciferase reporter control vector
  source 1..5708: mol_type = other DNA
taactataacggtcctaaggtagcgaacccccaactgagagaactcaaaggttaccccagttggggttaattaaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcgccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtgatgagaattcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctccgcgcccggggagcccaagggcacgccctggcacctagggggtatccccgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagt

SEQ ID NO: 32 - DNA, 7413 bp, synthetic construct
  misc_feature 1..7413: note = Synthetic: P006 GFP-dsREDE2 reporter construct comprising a cruciform structure
  source 1..7413: mol_type = other DNA
acgcgttgacattgattattgactagttaactataacggtcctaaggtagcgaacccccaactgagagaactcaaaggttaccccagttggggtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattcgaagcttgagctcgagatcctactagtaggatctgagtccggattacttgtacagctcgtccatgccgtgagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctgcaggaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggtttcctttcttacctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtagcgtgctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccgagctcggtaccacatgtaagcttcgaggggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcttaattaaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcgccaccatggatagcactgagaacgtcatcaagcccttcatgcgcttcaaggtgcacatggagggctccgtgaacggccacgagttcgagatcgagggcgagggcgagggcaagccctacgagggcacccagaccgccaagctgcaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggcaccttcatctaccacgtgaagttcatcggcgtgaacttcccctccgacggccccgtaatgcagaagaagactctgggctgggagccctccaccgagcgcctgtacccccgcgacggcgtgctgaagggcgagatccacaaggcgctgaagctgaagggcggcggccactacctggtggagttcaagtcaatctacatggccaagaagcccgtgaagctgcccggctactactacgtggactccaagctggacatcacctcccacaacgaggactacaccgtggtggagcagtacgagcgcgccgaggcccgccaccacctgttccagtgagaattcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctccgcgcccggggagcccaagggcacgccctggcacctagggggtatccccgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatat

SEQ ID NO: 33 - DNA, 6332 bp, synthetic construct
  misc_feature 1..6332: note = Synthetic: P020 sTNALP-Fc-(DSS)6 construct
  source 1..6332: mol_type = other DNA
taactataacggtcctaaggtagcgaacccccaactgagagaactcaaaggttaccccagttggggttaattaaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcgccaccatggatatgcgcgtacccgctcaacttctgggtttgttgctgctttggctcaggggagcccgctgtgccgcgcttgtccctgaaaaggagaaggaccccaaatattggagagaccaggctcaggaaaccctcaagtacgctcttgaactgcaaaagcttaatactaacgtggctaaaaacgtgataatgttcttgggggacggaatgggggtttcaaccgttaccgcggccagaatcttgaaagggcagctgcaccataacccaggcgaagaaacgaggctggagatggacaaatttccattcgtggcgctttctaagacctacaacactaacgcccaggtccctgactcagctggtacggccactgcctacctttgtggagtgaaggcaaatgaggggactgttggtgtttccgcagctactgagagatctcggtgtaatacgactcaaggtaatgaagttacatccatactccgatgggcaaaagacgcgggcaaatctgttgggattgttacaactactagggtgaaccatgcgacaccctctgcagcttatgcgcacagtgccgaccgagactggtattcagataatgagatgccacccgaggcccttagccagggctgcaaggacatagcctatcaattgatgcacaatatcagagacatagacgttataatgggtggtggtcgcaagtacatgtaccccaaaaacaaaaccgacgtagagtatgaatccgatgaaaaagctaggggcaccagactcgacggacttgatctggttgatacatggaagagcttcaagcccagatacaagcattcacatttcatctggaacaggaccgaacttctcacattggacccccacaatgttgactatctcctcggtttgtttgaaccaggggatatgcagtatgagctcaatcggaacaacgtcactgaccccagtttgagtgaaatggtcgttgttgcaatacaaattttgaggaaaaatccaaaggggtttttcttgttggtagagggcggacggatagatcacgggcaccacgaaggcaaagcaaaacaggccctgcacgaagccgttgaaatggatcgcgctatcggtcaagccggctccctgacgagtagtgaagataccttgaccgtcgtcactgcggatcatagccacgtttttacctttgggggatacacccccaggggcaactcaatttttggacttgcaccgatgctcagcgacacagataaaaagccttttactgcgattctgtatggcaacggccctggatacaaagtagtagggggtgaaagggaaaacgtcagcatggtggactatgcacacaataattatcaggcgcagtccgctgttccgctgcggcatgagactcacgggggagaggacgttgctgtcttttccaagggaccaatggctcatcttctccatggagtccatgagcaaaattatgtccctcatgtcatggcatacgctgcatgtataggagcaaatctcgggcactgtgcaccggcgtcctccctcaaagacaaaacacacacttgtccaccttgcccggcacctgaacttctgggcggcccgagcgtttttttgttccctcctaaacctaaggacacgctcatgatcagccgcacgccagaggttacctgtgtagtggtggatgtcagccacgaggatccggaagtaaaattcaactggtatgtagatggggtggaggttcacaatgccaaaaccaagccacgagaggaacagtacaatagtacttaccgggtcgtgtctgtattgaccgtcctgcatcaggattggttgaacgggaaagaatataagtgcaaggtgagcaacaaagcacttccggccccaatagagaaaacaattagtaaagcaaaaggacaaccacgggaaccgcaagtttatacactgccgccctctcgagacgagctgaccaaaaatcaggttagccttacatgtttggtcaaggggttctacccgagtgatattgcagtggaatgggaatccaacggtcaaccagaaaataactacaaaactactccgccggtgttggattcagacggctctttctttctgtatagcaaactcactgttgacaagtcaagatggcagcagggcaatgtatttagctgtagtgttatgcacgaagctcttcataaccactacacacaaaagtcactttctctttccccaggaaaagacatcgattccagcgacagttcagacagctctgacagctccgattcctccgactcaagttgatgagaattcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctccgcgcccggggagcccaagggcacgccctggcacctagggggtatccccgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagt

SEQ ID NO: 34 - DNA, 11405 bp, synthetic construct
  misc_feature 1..11405: note = Synthetic: Fw12-EF1a-sTNALP-Fc-(DSS)6 lentiviral construct
  source 1..11405: mol_type = other DNA
gtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcgccaccatggatatgcgcgtacccgctcaacttctgggtttgttgctgctttggctcaggggagcccgctgtgccgcgcttgtccctgaaaaggagaaggaccccaaatattggagagaccaggctcaggaaaccctcaagtacgctcttgaactgcaaaagcttaatactaacgtggctaaaaacgtgataatgttcttgggggacggaatgggggtttcaaccgttaccgcggccagaatcttgaaagggcagctgcaccataacccaggcgaagaaacgaggctggagatggacaaatttccattcgtggcgctttctaagacctacaacactaacgcccaggtccctgactcagctggtacggccactgcctacctttgtggagtgaaggcaaatgaggggactgttggtgtttccgcagctactgagagatctcggtgtaatacgactcaaggtaatgaagttacatccatactccgatgggcaaaagacgcgggcaaatctgttgggattgttacaactactagggtgaaccatgcgacaccctctgcagcttatgcgcacagtgccgaccgagactggtattcagataatgagatgccacccgaggcccttagccagggctgcaaggacatagcctatcaattgatgcacaatatcagagacatagacgttataatgggtggtggtcgcaagtacatgtaccccaaaaacaaaaccgacgtagagtatgaatccgatgaaaaagctaggggcaccagactcgacggacttgatctggttgatacatggaagagcttcaagcccagatacaagcattcacatttcatctggaacaggaccgaacttctcacattggacccccacaatgttgactatctcctcggtttgtttgaaccaggggatatgcagtatgagctcaatcggaacaacgtcactgaccccagtttgagtgaaatggtcgttgttgcaatacaaattttgaggaaaaatccaaaggggtttttcttgttggtagagggcggacggatagatcacgggcaccacgaaggcaaagcaaaacaggccctgcacgaagccgttgaaatggatcgcgctatcggtcaagccggctccctgacgagtagtgaagataccttgaccgtcgtcactgcggatcatagccacgtttttacctttgggggatacacccccaggggcaactcaatttttggacttgcaccgatgctcagcgacacagataaaaagccttttactgcgattctgtatggcaacggccctggatacaaagtagtagggggtgaaagggaaaacgtcagcatggtggactatgcacacaataattatcaggcgcagtccgctgttccgctgcggcatgagactcacgggggagaggacgttgctgtcttttccaagggaccaatggctcatcttctccatggagtccatgagcaaaattatgtccctcatgtcatggcatacgctgcatgtataggagcaaatctcgggcactgtgcaccggcgtcctccctcaaagacaaaacacacacttgtccaccttgcccggcacctgaacttctgggcggcccgagcgtttttttgttccctcctaaacctaaggacacgctcatgatcagccgcacgccagaggttacctgtgtagtggtggatgtcagccacgaggatccggaagtaaaattcaactggtatgtagatggggtggaggttcacaatgccaaaaccaagccacgagaggaacagtacaatagtacttaccgggtcgtgtctgtattgaccgtcctgcatcaggattggttgaacgggaaagaatataagtgcaaggtgagcaacaaagcacttccggccccaatagagaaaacaattagtaaagcaaaaggacaaccacgggaaccgcaagtttatacactgccgccctctcgagacgagctgaccaaaaatcaggttagccttacatgtttggtcaaggggttctacccgagtgatattgcagtggaatgggaatccaacggtcaaccagaaaataactacaaaactactccgccggtgttggattcagacggctctttctttctgtatagcaaactcactgttgacaagtcaagatggcagcagggcaatgtatttagctgtagtgttatgcacgaagctcttcataaccactacacacaaaagtcactttctctttccccaggaaaagacatcgattccagcgacagttcagacagctctgacagctccgattcctccgactcaagttgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcct

SEQ ID NO: 35 - DNA, 709 bp, synthetic construct
  misc_feature 1..709: note = Synthetic: Two inverted repeat sequences separated by a minimal R6K gamma origin
  source 1..709: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaataccgttcgtataatgtatgctatacgaacggtactgcagtgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 36 - DNA, 128 bp, synthetic construct
  misc_feature 1..128: note = Synthetic: AAV2 inverted
  source 1..128: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 37 - DNA, 166 bp, synthetic construct
  misc_feature 1..166: note = Synthetic: AAV2 A CRUCIFORM structure with a DD element
  source 1..166: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggtttcct

SEQ ID NO: 38 - DNA, 4681 bp, synthetic construct
  misc_feature 1..4681: note = Synthetic: MF-EF1a-sTNALP control construct
  source 1..4681: mol_type = other DNA
gcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcgccaccatggatatgcgcgtacccgctcaacttctgggtttgttgctgctttggctcaggggagcccgctgtgccgcgcttgtccctgaaaaggagaaggaccccaaatattggagagaccaggctcaggaaaccctcaagtacgctcttgaactgcaaaagcttaatactaacgtggctaaaaacgtgataatgttcttgggggacggaatgggggtttcaaccgttaccgcggccagaatcttgaaagggcagctgcaccataacccaggcgaagaaacgaggctggagatggacaaatttccattcgtggcgctttctaagacctacaacactaacgcccaggtccctgactcagctggtacggccactgcctacctttgtggagtgaaggcaaatgaggggactgttggtgtttccgcagctactgagagatctcggtgtaatacgactcaaggtaatgaagttacatccatactccgatgggcaaaagacgcgggcaaatctgttgggattgttacaactactagggtgaaccatgcgacaccctctgcagcttatgcgcacagtgccgaccgagactggtattcagataatgagatgccacccgaggcccttagccagggctgcaaggacatagcctatcaattgatgcacaatatcagagacatagacgttataatgggtggtggtcgcaagtacatgtaccccaaaaacaaaaccgacgtagagtatgaatccgatgaaaaagctaggggcaccagactcgacggacttgatctggttgatacatggaagagcttcaagcccagatacaagcattcacatttcatctggaacaggaccgaacttctcacattggacccccacaatgttgactatctcctcggtttgtttgaaccaggggatatgcagtatgagctcaatcggaacaacgtcactgaccccagtttgagtgaaatggtcgttgttgcaatacaaattttgaggaaaaatccaaaggggtttttcttgttggtagagggcggacggatagatcacgggcaccacgaaggcaaagcaaaacaggccctgcacgaagccgttgaaatggatcgcgctatcggtcaagccggctccctgacgagtagtgaagataccttgaccgtcgtcactgcggatcatagccacgtttttacctttgggggatacacccccaggggcaactcaatttttggacttgcaccgatgctcagcgacacagataaaaagccttttactgcgattctgtatggcaacggccctggatacaaagtagtagggggtgaaagggaaaacgtcagcatggtggactatgcacacaataattatcaggcgcagtccgctgttccgctgcggcatgagactcacgggggagaggacgttgctgtcttttccaagggaccaatggctcatcttctccatggagtccatgagcaaaattatgtccctcatgtcatggcatacgctgcatgtataggagcaaatctcgggcactgtgcaccggcgtcctccctcaaagacaaaacacacacttgtccaccttgcccggcacctgaacttctgggcggcccgagcgtttttttgttccctcctaaacctaaggacacgctcatgatcagccgcacgccagaggttacctgtgtagtggtggatgtcagccacgaggatccggaagtaaaattcaactggtatgtagatggggtggaggttcacaatgccaaaaccaagccacgagaggaacagtacaatagtacttaccgggtcgtgtctgtattgaccgtcctgcatcaggattggttgaacgggaaagaatataagtgcaaggtgagcaacaaagcacttccggccccaatagagaaaacaattagtaaagcaaaaggacaaccacgggaaccgcaagtttatacactgccgccctctcgagacgagctgaccaaaaatcaggttagccttacatgtttggtcaaggggttctacccgagtgatattgcagtggaatgggaatccaacggtcaaccagaaaataactacaaaactactccgccggtgttggattcagacggctctttctttctgtatagcaaactcactgttgacaagtcaagatggcagcagggcaatgtatttagctgtagtgttatgcacgaagctcttcataaccactacacacaaaagtcactttctctttccccaggaaaagacatcgattccagcgacagttcagacagctctgacagctccgattcctccgactcaagttgaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggataccgttcgtataatgtatgctatacgaacggtactgcagtgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcttaattaaggggctccggtgcccgtcagtgggcagagcgcacatcgcccacagtccccgagaagttggggggaggggtcggcaattgaaccggt

SEQ ID NO: 39 - DNA, 30 bp, synthetic construct
  misc_feature 1..30: note = Synthetic: QTS - TLR9 inhibitory sequence
  source 1..30: mol_type = other DNA
ttagggttagggttagggttagggttaggg

SEQ ID NO: 40 - DNA, 5148 bp, synthetic construct
  misc_feature 1..5148: note = Synthetic: Halo construct comprising R6K Ori sequence between two inverted repeated sequences, one TLR9 inhibitory sequence, EF1a-driven sTNALP-Fc-(DSS)6 transgene
  source 1..5148: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcttagggttagggttagggttagggttaggggcccttaagctagcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcgccaccatggatatgcgcgtacccgctcaacttctgggtttgttgctgctttggctcaggggagcccgctgtgccgcgcttgtccctgaaaaggagaaggaccccaaatattggagagaccaggctcaggaaaccctcaagtacgctcttgaactgcaaaagcttaatactaacgtggctaaaaacgtgataatgttcttgggggacggaatgggggtttcaaccgttaccgcggccagaatcttgaaagggcagctgcaccataacccaggcgaagaaacgaggctggagatggacaaatttccattcgtggcgctttctaagacctacaacactaacgcccaggtccctgactcagctggtacggccactgcctacctttgtggagtgaaggcaaatgaggggactgttggtgtttccgcagctactgagagatctcggtgtaatacgactcaaggtaatgaagttacatccatactccgatgggcaaaagacgcgggcaaatctgttgggattgttacaactactagggtgaaccatgcgacaccctctgcagcttatgcgcacagtgccgaccgagactggtattcagataatgagatgccacccgaggcccttagccagggctgcaaggacatagcctatcaattgatgcacaatatcagagacatagacgttataatgggtggtggtcgcaagtacatgtaccccaaaaacaaaaccgacgtagagtatgaatccgatgaaaaagctaggggcaccagactcgacggacttgatctggttgatacatggaagagcttcaagcccagatacaagcattcacatttcatctggaacaggaccgaacttctcacattggacccccacaatgttgactatctcctcggtttgtttgaaccaggggatatgcagtatgagctcaatcggaacaacgtcactgaccccagtttgagtgaaatggtcgttgttgcaatacaaattttgaggaaaaatccaaaggggtttttcttgttggtagagggcggacggatagatcacgggcaccacgaaggcaaagcaaaacaggccctgcacgaagccgttgaaatggatcgcgctatcggtcaagccggctccctgacgagtagtgaagataccttgaccgtcgtcactgcggatcatagccacgtttttacctttgggggatacacccccaggggcaactcaatttttggacttgcaccgatgctcagcgacacagataaaaagccttttactgcgattctgtatggcaacggccctggatacaaagtagtagggggtgaaagggaaaacgtcagcatggtggactatgcacacaataattatcaggcgcagtccgctgttccgctgcggcatgagactcacgggggagaggacgttgctgtcttttccaagggaccaatggctcatcttctccatggagtccatgagcaaaattatgtccctcatgtcatggcatacgctgcatgtataggagcaaatctcgggcactgtgcaccggcgtcctccctcaaagacaaaacacacacttgtccaccttgcccggcacctgaacttctgggcggcccgagcgtttttttgttccctcctaaacctaaggacacgctcatgatcagccgcacgccagaggttacctgtgtagtggtggatgtcagccacgaggatccggaagtaaaattcaactggtatgtagatggggtggaggttcacaatgccaaaaccaagccacgagaggaacagtacaatagtacttaccgggtcgtgtctgtattgaccgtcctgcatcaggattggttgaacgggaaagaatataagtgcaaggtgagcaacaaagcacttccggccccaatagagaaaacaattagtaaagcaaaaggacaaccacgggaaccgcaagtttatacactgccgccctctcgagacgagctgaccaaaaatcaggttagccttacatgtttggtcaaggggttctacccgagtgatattgcagtggaatgggaatccaacggtcaaccagaaaataactacaaaactactccgccggtgttggattcagacggctctttctttctgtatagcaaactcactgttgacaagtcaagatggcagcagggcaatgtatttagctgtagtgttatgcacgaagctcttcataaccactacacacaaaagtcactttctctttccccaggaaaagacatcgattccagcgacagttcagacagctctgacagctccgattcctccgactcaagttgaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaataccgttcgtataatgtatgctatacgaacggtactgcagtgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcttaattaagg

SEQ ID NO: 41 - DNA, 5178 bp, synthetic construct
  misc_feature 1..5178: note = Synthetic: Halo construct comprising ITR-R6K Ori-ITR sequence, two TLR9 inhibitory sequences, EF1a-driven sTNALP-Fc-(DSS)6 transgene
  source 1..5178: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcttagggttagggttagggttagggttaggggcccttaagctagcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcgccaccatggatatgcgcgtacccgctcaacttctgggtttgttgctgctttggctcaggggagcccgctgtgccgcgcttgtccctgaaaaggagaaggaccccaaatattggagagaccaggctcaggaaaccctcaagtacgctcttgaactgcaaaagcttaatactaacgtggctaaaaacgtgataatgttcttgggggacggaatgggggtttcaaccgttaccgcggccagaatcttgaaagggcagctgcaccataacccaggcgaagaaacgaggctggagatggacaaatttccattcgtggcgctttctaagacctacaacactaacgcccaggtccctgactcagctggtacggccactgcctacctttgtggagtgaaggcaaatgaggggactgttggtgtttccgcagctactgagagatctcggtgtaatacgactcaaggtaatgaagttacatccatactccgatgggcaaaagacgcgggcaaatctgttgggattgttacaactactagggtgaaccatgcgacaccctctgcagcttatgcgcacagtgccgaccgagactggtattcagataatgagatgccacccgaggcccttagccagggctgcaaggacatagcctatcaattgatgcacaatatcagagacatagacgttataatgggtggtggtcgcaagtacatgtaccccaaaaacaaaaccgacgtagagtatgaatccgatgaaaaagctaggggcaccagactcgacggacttgatctggttgatacatggaagagcttcaagcccagatacaagcattcacatttcatctggaacaggaccgaacttctcacattggacccccacaatgttgactatctcctcggtttgtttgaaccaggggatatgcagtatgagctcaatcggaacaacgtcactgaccccagtttgagtgaaatggtcgttgttgcaatacaaattttgaggaaaaatccaaaggggtttttcttgttggtagagggcggacggatagatcacgggcaccacgaaggcaaagcaaaacaggccctgcacgaagccgttgaaatggatcgcgctatcggtcaagccggctccctgacgagtagtgaagataccttgaccgtcgtcactgcggatcatagccacgtttttacctttgggggatacacccccaggggcaactcaatttttggacttgcaccgatgctcagcgacacagataaaaagccttttactgcgattctgtatggcaacggccctggatacaaagtagtagggggtgaaagggaaaacgtcagcatggtggactatgcacacaataattatcaggcgcagtccgctgttccgctgcggcatgagactcacgggggagaggacgttgctgtcttttccaagggaccaatggctcatcttctccatggagtccatgagcaaaattatgtccctcatgtcatggcatacgctgcatgtataggagcaaatctcgggcactgtgcaccggcgtcctccctcaaagacaaaacacacacttgtccaccttgcccggcacctgaacttctgggcggcccgagcgtttttttgttccctcctaaacctaaggacacgctcatgatcagccgcacgccagaggttacctgtgtagtggtggatgtcagccacgaggatccggaagtaaaattcaactggtatgtagatggggtggaggttcacaatgccaaaaccaagccacgagaggaacagtacaatagtacttaccgggtcgtgtctgtattgaccgtcctgcatcaggattggttgaacgggaaagaatataagtgcaaggtgagcaacaaagcacttccggccccaatagagaaaacaattagtaaagcaaaaggacaaccacgggaaccgcaagtttatacactgccgccctctcgagacgagctgaccaaaaatcaggttagccttacatgtttggtcaaggggttctacccgagtgatattgcagtggaatgggaatccaacggtcaaccagaaaataactacaaaactactccgccggtgttggattcagacggctctttctttctgtatagcaaactcactgttgacaagtcaagatggcagcagggcaatgtatttagctgtagtgttatgcacgaagctcttcataaccactacacacaaaagtcactttctctttccccaggaaaagacatcgattccagcgacagttcagacagctctgacagctccgattcctccgactcaagttgaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagtttagggttagggttagggttagggttagggtaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaataccgttcgtataatgtatgctatacgaacggtactgcagtgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcttaattaagg

SEQ ID NO: 42 - DNA, 4932 bp, synthetic construct
  misc_feature 1..4932: note = Synthetic: Halo construct comprising ITR-R6K Ori-ITR sequence, EF1a-driven Zero-CpG PCSK9 Q152H transgene
  source 1..4932: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcgccaccatgggcacagtgagctccagaagatcttggtggcctctgcctctgctgctgctcctgctgctgctgctgggacctgctggagctagagcccaggaggatgaagatggagactatgaggagctggtgctggctctgagatctgaggaagatggtctggctgaagcccctgagcatggcaccacagctacctttcacagatgtgccaaggacccctggagactgcctggcacctatgtggtggttcttaaagaggagacacatctgagccagtctgagagaacagccagaagactgcaggcccaagctgctagaagaggttatctgaccaagatcctgcatgtgtttcatggccttcttcctggcttcctggtcaagatgtctggagatctgcttgagctggccctgaagctgccccatgtggattacattgaggaggatagctctgtgtttgcccactctattccttggaacctggaaagaatcacccctccaagatatagagctgatgaataccagcctcctgatggaggcagcctggtggaggtgtacctgctggacaccagcatccagtctgaccacagggagattgagggcagagttatggtgacagattttgagaatgtgccagaagaggatggcaccagattccacagacaagccagcaagtgtgacagccatggcacccacctggctggagtggtgtctggcagagatgctggagtggccaagggagccagcatgagaagcctgagagtgctgaattgccagggcaagggcacagtgtctggcaccctaattggactggagttcatcagaaagagccagctggtgcagcctgtgggacctctggttgttctgctgcctctagctggaggatacagcagagtgctgaatgctgcctgccagagactggctagagctggagtggtgctggtgactgctgctggaaatttcagagatgatgcctgcctgtacagccctgcctctgcccctgaagtgatcacagtgggagctacaaatgcccaggaccaaccagtgaccctgggcaccctgggaaccaactttggcagatgtgtggacctgtttgcccctggagaggacatcattggagccagctcagattgctctacatgctttgtgagccaatctggcacctcacaggctgctgctcatgtggctggcattgctgccatgatgctgtctgcagaacctgagctgacactggctgagctgagacagagactgatccacttctctgctaaagatgtgatcaatgaggcctggttccctgaagaccagagagtgctgacacctaatctggtggctgctctgcctcccagcacccatggagctggatggcagctgttctgcagaacagtgtggtctgcccactctggccccaccagaatggccacagctgtggccagatgtgcccctgatgaggaactgctcagctgcagcagctttagcagatctggcaaaagaagaggagaaagaatggaagcccagggaggcaagctggtgtgcagagcccacaatgcctttggaggagaaggagtgtatgccattgccagatgttgcctgctgccccaggccaactgctctgtgcacacagcccctccagctgaggcctctatgggcaccagagtgcactgccaccagcagggccatgtgctgacaggctgttctagccattgggaggtggaagacctgggaacccacaagcctcctgtgctgagacccagaggccagcctaaccagtgtgtgggccacagagaagccagcatccatgccagttgttgtcatgcccctggcctggaatgcaaggtgaaggaacatggcatcccagctccacaggagcaggtgacagtggcctgtgaggaaggctggaccctgacaggctgctctgccctgcctggcacaagccatgtgctgggagcatatgctgtggacaacacctgtgtggtgagaagcagggatgtgagcacaacaggcagcacctctgagggagctgttacagctgtggccatctgctgtagatctagacacttggcccaggcctctcaggagctgcagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaataccgttcgtataatgtatgctatacgaacggtactgcagtgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcttaattaagg

SEQ ID NO: 43 - DNA, 4371 bp, synthetic construct
  misc_feature 1..4371: note = Synthetic: Halo construct comprising IR-R6K Ori-IR sequence, and EF1a-driven cynomolgus monkey sTNALP transgene
  source 1..4371: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcgccaccatggacatgagagtgcctgcccagctgctgggcctgctgctgctgtggctgagaggagccagatgtgctgccctggtgcctgagaaggagaaggaccccaagtactggagagaccaggcccaggagaccctgaagtatgccctggagctgcagaagctgaacaccaatgtggccaagaatgtgatcatgttcctgggagatggcatgggagtgagcacagtgacagccaccagaatcctgaagggccagctgcaccacaaccctggagaggagaccagactggagatggacaagttcccctttgtggccctgagcaagacctacaacaccaatgcccaggtgcctgactctgctggcacagccacagcctacctgtgtggagtgaaggccaatgagggcacagtgggagtgtctgctgccacagagagaagcagatgcaacaccacccagggcaatgaggtgaccagcatcctgagatgggccaaggatgctggcaagtctgtgggcattgtgaccaccaccagagtgaaccatgccaccccctctgctgcctatgcccactctgctgacagagactggtactctgacaatgagatgccccctgaggccctgagccagggctgcaaggacattgcctaccagctggtgcacaacatcagagacattgatgtgatcatgggaggaggcagaaagtacatgtaccccaagaacaagacagatgtggagtatgagattgatgagaaggccagaggcaccagactggatggcctggacctggtgaacatctggaagagcttcaagcccagacacaagcacagccacttcatctggaacagaacagagctgctgaccctggacccccacaatgtggactacctgctgggcctgtttgagcctggagacatggagtatgagctgaacagaaacaatgtgacagaccccagcctgtctgagatggtggtggtggccatccagatcctgagaaagaaccccaagggcttcttcctgctggtggagggaggcagaattgaccatggccaccatgagggcaaggccaagcaggccctgcatgaggctgtggagatggacagagccattggccaggctggcagcatgaccagcctggaggacaccctgacagtggtgacagctgaccacagccatgtgttcacctttggaggctacacccccagaggcaacagcatctttggcctggcccccatgctgtctgacacagacaagaagcccttcacagccatcctgtatggcaatggccctggctacaaggtggtgggaggagagagagagaatgtgagcatggtggactatgcccacaacaactaccaggcccagtctgctgtgcccctgagacatgagacccatggaggagaggatgtggctgtgttcagcaagggccccatggcccacctgctgcatggagtgcatgagcagaactacatcccccatgtgatggcctatgctgcctgcattggagccaacctggaccactgtgcccctgccagcagctgaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaataccgttcgtataatgtatgctatacgaacggtactgcagtgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcttaattaagg

SEQ ID NO: 44 - DNA, 4016 bp, synthetic construct
  misc_feature 1..4016: note = Synthetic: Halo construct comprising A a cruiciform structure with a DD elementCRUCIFORM, and human TBG promoter-driven human sTNALP-Fc-(DSS)6 transgene
  source 1..4016: mol_type = other DNA
ctaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggtttcctttaattaacttaagtgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaatttaaataggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtgctagcccaccatggatatgagagtacctgctcaacttctgggtttgttgctgctttggctcaggggagccagatgtgctgccctggttcctgagaaggaaaaggaccccaagtactggagagatcaggcacaggagacactgaagtatgccctggagctgcagaagctgaataccaatgttgccaaaaatgtgatcatgttcctgggagatgggatgggagtcagcacagtgacagctgctagaatcctgaagggacagctgcatcataatcctggagaagagaccagactggaaatggataagttcccctttgtggccctgagcaagacatataataccaatgcccaagtgcctgattctgctggaacagctacagcttacctgtgtggagtgaaggccaatgagggaacagtgggagtgtctgctgccacagaaagaagcagatgcaacaccacccaaggcaatgaggtgacaagcatcctgagatgggccaaggatgctggcaaatctgtgggcattgtgacaacaaccagagtgaaccatgccaccccatctgctgcttatgcccattctgctgacagagactggtactctgacaatgagatgcctccagaggctctgtcccagggttgtaaagatattgcctatcagctgatgcacaacatcagagacattgatgtgatcatgggaggaggcaggaagtacatgtaccccaagaacaagacagatgtggagtatgagtctgatgagaaggccagaggcacaagactggatggcctggacctggtggacacctggaagtcttttaagcccagatacaagcacagccacttcatctggaacagaacagaactgctgaccctggacccccacaatgtggactacctgctgggcctgtttgagcctggagatatgcagtatgagctgaatagaaacaatgtgacagacccctccctgtctgaaatggtggtggtggccatccagatcctgagaaagaaccctaagggctttttcctgctggttgagggaggaagaattgaccatggccatcatgagggcaaggctaagcaggccctgcatgaggctgtggagatggacagagccattggccaagctggctctctgaccagctcagaggacaccctgacagtggtgacagctgatcactctcatgtgttcacttttggaggatatacccctagaggcaacagcatctttggcctggcccctatgctgtctgacacagacaaaaagcccttcacagccatcctgtatggcaatggcccaggatacaaggtggtgggaggagagagagagaatgtgtctatggtggactatgcacacaacaactaccaggcccagtctgctgttcctctgagacatgaaacacatggaggggaagatgtggcagtgttttctaaaggccctatggctcacctgctgcatggagtccatgagcagaactatgtgccccatgtgatggcctatgctgcctgcattggagcaaacctgggccactgtgcccctgccagcagcctgaaagacaagacccacacatgtcctccctgccctgcccctgagctgctgggaggaccttctgtgttcctgttccctccaaagcctaaagacaccctgatgatcagcagaacccctgaagtgacttgtgtggtggtggatgtcagccatgaagatccagaagtgaaattcaactggtatgtggatggagtggaggtgcacaatgccaaaaccaagcctagagaagaacagtacaacagcacctacagagtggtgtctgtgctgacagtgcttcaccaggactggctgaatggcaaggaatacaagtgcaaggtgagcaacaaggccctgcctgctcctattgagaagaccatcagcaaggccaagggacagcccagagaacctcaggtctacaccctgcctcctagcagagatgagctgacaaagaaccaggtgagcctgacatgcctggtgaagggcttctacccttctgatattgctgtggaatgggagtctaatggccaacctgaaaacaactacaagacaacaccccctgtgctggactctgatggcagcttcttcctgtactccaaactgacagtggacaagtccagatggcagcagggcaatgtgttcagttgctctgtcatgcatgaagccctgcacaaccactacacccagaaaagcctcagcctgagccctggaaaggatattgactcctctgattcttctgacagctcagactcctctgatagctctgacagctcttgaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggataccgttcgtataatgtatgctatacgaacggtactgcagtgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcttaattaagg

SEQ ID NO: 45 - DNA, 4266 bp, synthetic construct
  misc_feature 1..4266: note = Synthetic: Halo construct comprising IR-R6K Ori-IR sequence, and human TBG liver-specific promoter-driven Zero-CpG sTNALP-Fc-(DSS)6 transgene
  source 1..4266: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagtgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaatttaaataggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtgctagcccaccatggatatgagagtacctgctcaacttctgggtttgttgctgctttggctcaggggagccagatgtgctgccctggttcctgagaaggaaaaggaccccaagtactggagagatcaggcacaggagacactgaagtatgccctggagctgcagaagctgaataccaatgttgccaaaaatgtgatcatgttcctgggagatgggatgggagtcagcacagtgacagctgctagaatcctgaagggacagctgcatcataatcctggagaagagaccagactggaaatggataagttcccctttgtggccctgagcaagacatataataccaatgcccaagtgcctgattctgctggaacagctacagcttacctgtgtggagtgaaggccaatgagggaacagtgggagtgtctgctgccacagaaagaagcagatgcaacaccacccaaggcaatgaggtgacaagcatcctgagatgggccaaggatgctggcaaatctgtgggcattgtgacaacaaccagagtgaaccatgccaccccatctgctgcttatgcccattctgctgacagagactggtactctgacaatgagatgcctccagaggctctgtcccagggttgtaaagatattgcctatcagctgatgcacaacatcagagacattgatgtgatcatgggaggaggcaggaagtacatgtaccccaagaacaagacagatgtggagtatgagtctgatgagaaggccagaggcacaagactggatggcctggacctggtggacacctggaagtcttttaagcccagatacaagcacagccacttcatctggaacagaacagaactgctgaccctggacccccacaatgtggactacctgctgggcctgtttgagcctggagatatgcagtatgagctgaatagaaacaatgtgacagacccctccctgtctgaaatggtggtggtggccatccagatcctgagaaagaaccctaagggctttttcctgctggttgagggaggaagaattgaccatggccatcatgagggcaaggctaagcaggccctgcatgaggctgtggagatggacagagccattggccaagctggctctctgaccagctcagaggacaccctgacagtggtgacagctgatcactctcatgtgttcacttttggaggatatacccctagaggcaacagcatctttggcctggcccctatgctgtctgacacagacaaaaagcccttcacagccatcctgtatggcaatggcccaggatacaaggtggtgggaggagagagagagaatgtgtctatggtggactatgcacacaacaactaccaggcccagtctgctgttcctctgagacatgaaacacatggaggggaagatgtggcagtgttttctaaaggccctatggctcacctgctgcatggagtccatgagcagaactatgtgccccatgtgatggcctatgctgcctgcattggagcaaacctgggccactgtgcccctgccagcagcctgaaagacaagacccacacatgtcctccctgccctgcccctgagctgctgggaggaccttctgtgttcctgttccctccaaagcctaaagacaccctgatgatcagcagaacccctgaagtgacttgtgtggtggtggatgtcagccatgaagatccagaagtgaaattcaactggtatgtggatggagtggaggtgcacaatgccaaaaccaagcctagagaagaacagtacaacagcacctacagagtggtgtctgtgctgacagtgcttcaccaggactggctgaatggcaaggaatacaagtgcaaggtgagcaacaaggccctgcctgctcctattgagaagaccatcagcaaggccaagggacagcccagagaacctcaggtctacaccctgcctcctagcagagatgagctgacaaagaaccaggtgagcctgacatgcctggtgaagggcttctacccttctgatattgctgtggaatgggagtctaatggccaacctgaaaacaactacaagacaacaccccctgtgctggactctgatggcagcttcttcctgtactccaaactgacagtggacaagtccagatggcagcagggcaatgtgttcagttgctctgtcatgcatgaagccctgcacaaccactacacccagaaaagcctcagcctgagccctggaaaggatattgactcctctgattcttctgacagctcagactcctctgatagctctgacagctcttgaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaataccgttcgtataatgtatgctatacgaacggtactgcagtgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcttaattaagg

SEQ ID NO: 46 - DNA, 4016 bp, synthetic construct
  misc_feature 1..4016: note = Synthetic: Halo construct comprising Zero-CpG A CRUCIFORM variant 1, and human TBG liver-specific promoter-driven Zero-CpG human sTNALP-Fc-(DSS)6 transgene
  source 1..4016: mol_type = other DNA
ttaattaaggctaggaacccctagtgatggagttggccactccctctctgccctctccctctctcactgaggcctggggaccaaaggtccccagactgcagagctttgctctgcaggcctcagtgagagagggagagggcagagagggagtggccaactccatcactaggggtttcctttaattaacttaagtgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaatttaaataggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtgctagcccaccatggatatgagagtacctgctcaacttctgggtttgttgctgctttggctcaggggagccagatgtgctgccctggttcctgagaaggaaaaggaccccaagtactggagagatcaggcacaggagacactgaagtatgccctggagctgcagaagctgaataccaatgttgccaaaaatgtgatcatgttcctgggagatgggatgggagtcagcacagtgacagctgctagaatcctgaagggacagctgcatcataatcctggagaagagaccagactggaaatggataagttcccctttgtggccctgagcaagacatataataccaatgcccaagtgcctgattctgctggaacagctacagcttacctgtgtggagtgaaggccaatgagggaacagtgggagtgtctgctgccacagaaagaagcagatgcaacaccacccaaggcaatgaggtgacaagcatcctgagatgggccaaggatgctggcaaatctgtgggcattgtgacaacaaccagagtgaaccatgccaccccatctgctgcttatgcccattctgctgacagagactggtactctgacaatgagatgcctccagaggctctgtcccagggttgtaaagatattgcctatcagctgatgcacaacatcagagacattgatgtgatcatgggaggaggcaggaagtacatgtaccccaagaacaagacagatgtggagtatgagtctgatgagaaggccagaggcacaagactggatggcctggacctggtggacacctggaagtcttttaagcccagatacaagcacagccacttcatctggaacagaacagaactgctgaccctggacccccacaatgtggactacctgctgggcctgtttgagcctggagatatgcagtatgagctgaatagaaacaatgtgacagacccctccctgtctgaaatggtggtggtggccatccagatcctgagaaagaaccctaagggctttttcctgctggttgagggaggaagaattgaccatggccatcatgagggcaaggctaagcaggccctgcatgaggctgtggagatggacagagccattggccaagctggctctctgaccagctcagaggacaccctgacagtggtgacagctgatcactctcatgtgttcacttttggaggatatacccctagaggcaacagcatctttggcctggcccctatgctgtctgacacagacaaaaagcccttcacagccatcctgtatggcaatggcccaggatacaaggtggtgggaggagagagagagaatgtgtctatggtggactatgcacacaacaactaccaggcccagtctgctgttcctctgagacatgaaacacatggaggggaagatgtggcagtgttttctaaaggccctatggctcacctgctgcatggagtccatgagcagaactatgtgccccatgtgatggcctatgctgcctgcattggagcaaacctgggccactgtgcccctgccagcagcctgaaagacaagacccacacatgtcctccctgccctgcccctgagctgctgggaggaccttctgtgttcctgttccctccaaagcctaaagacaccctgatgatcagcagaacccctgaagtgacttgtgtggtggtggatgtcagccatgaagatccagaagtgaaattcaactggtatgtggatggagtggaggtgcacaatgccaaaaccaagcctagagaagaacagtacaacagcacctacagagtggtgtctgtgctgacagtgcttcaccaggactggctgaatggcaaggaatacaagtgcaaggtgagcaacaaggccctgcctgctcctattgagaagaccatcagcaaggccaagggacagcccagagaacctcaggtctacaccctgcctcctagcagagatgagctgacaaagaaccaggtgagcctgacatgcctggtgaagggcttctacccttctgatattgctgtggaatgggagtctaatggccaacctgaaaacaactacaagacaacaccccctgtgctggactctgatggcagcttcttcctgtactccaaactgacagtggacaagtccagatggcagcagggcaatgtgttcagttgctctgtcatgcatgaagccctgcacaaccactacacccagaaaagcctcagcctgagccctggaaaggatattgactcctctgattcttctgacagctcagactcctctgatagctctgacagctcttgaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggataccgttcgtataatgtatgctatacgaacggtactgcagtgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatc

SEQ ID NO: 47 - DNA, 4016 bp, synthetic construct
  misc_feature 1..4016: note = Synthetic: Halo construct comprising Zero-CpG A CRUCIFORM structure variant 3, and human TBG liver-specific promoter-driven Zero-CpG human sTNALP-Fc-(DSS)6 transgene
  source 1..4016: mol_type = other DNA
ttaattaaggctaggaacccctagtgatggagttggccactccctctctgctcactcactcactcactgaggccagggcaccaaaggtgccctgacagccctgctttgcagggctggcctcagtgagtgagtgagtgagcagagagggagtggccaactccatcactaggggtttcctttaattaacttaagtgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaatttaaataggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtgctagcccaccatggatatgagagtacctgctcaacttctgggtttgttgctgctttggctcaggggagccagatgtgctgccctggttcctgagaaggaaaaggaccccaagtactggagagatcaggcacaggagacactgaagtatgccctggagctgcagaagctgaataccaatgttgccaaaaatgtgatcatgttcctgggagatgggatgggagtcagcacagtgacagctgctagaatcctgaagggacagctgcatcataatcctggagaagagaccagactggaaatggataagttcccctttgtggccctgagcaagacatataataccaatgcccaagtgcctgattctgctggaacagctacagcttacctgtgtggagtgaaggccaatgagggaacagtgggagtgtctgctgccacagaaagaagcagatgcaacaccacccaaggcaatgaggtgacaagcatcctgagatgggccaaggatgctggcaaatctgtgggcattgtgacaacaaccagagtgaaccatgccaccccatctgctgcttatgcccattctgctgacagagactggtactctgacaatgagatgcctccagaggctctgtcccagggttgtaaagatattgcctatcagctgatgcacaacatcagagacattgatgtgatcatgggaggaggcaggaagtacatgtaccccaagaacaagacagatgtggagtatgagtctgatgagaaggccagaggcacaagactggatggcctggacctggtggacacctggaagtcttttaagcccagatacaagcacagccacttcatctggaacagaacagaactgctgaccctggacccccacaatgtggactacctgctgggcctgtttgagcctggagatatgcagtatgagctgaatagaaacaatgtgacagacccctccctgtctgaaatggtggtggtggccatccagatcctgagaaagaaccctaagggctttttcctgctggttgagggaggaagaattgaccatggccatcatgagggcaaggctaagcaggccctgcatgaggctgtggagatggacagagccattggccaagctggctctctgaccagctcagaggacaccctgacagtggtgacagctgatcactctcatgtgttcacttttggaggatatacccctagaggcaacagcatctttggcctggcccctatgctgtctgacacagacaaaaagcccttcacagccatcctgtatggcaatggcccaggatacaaggtggtgggaggagagagagagaatgtgtctatggtggactatgcacacaacaactaccaggcccagtctgctgttcctctgagacatgaaacacatggaggggaagatgtggcagtgttttctaaaggccctatggctcacctgctgcatggagtccatgagcagaactatgtgccccatgtgatggcctatgctgcctgcattggagcaaacctgggccactgtgcccctgccagcagcctgaaagacaagacccacacatgtcctccctgccctgcccctgagctgctgggaggaccttctgtgttcctgttccctccaaagcctaaagacaccctgatgatcagcagaacccctgaagtgacttgtgtggtggtggatgtcagccatgaagatccagaagtgaaattcaactggtatgtggatggagtggaggtgcacaatgccaaaaccaagcctagagaagaacagtacaacagcacctacagagtggtgtctgtgctgacagtgcttcaccaggactggctgaatggcaaggaatacaagtgcaaggtgagcaacaaggccctgcctgctcctattgagaagaccatcagcaaggccaagggacagcccagagaacctcaggtctacaccctgcctcctagcagagatgagctgacaaagaaccaggtgagcctgacatgcctggtgaagggcttctacccttctgatattgctgtggaatgggagtctaatggccaacctgaaaacaactacaagacaacaccccctgtgctggactctgatggcagcttcttcctgtactccaaactgacagtggacaagtccagatggcagcagggcaatgtgttcagttgctctgtcatgcatgaagccctgcacaaccactacacccagaaaagcctcagcctgagccctggaaaggatattgactcctctgattcttctgacagctcagactcctctgatagctctgacagctcttgaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggataccgttcgtataatgtatgctatacgaacggtactgcagtgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatc

SEQ ID NO: 48 - DNA, 4244 bp, synthetic construct
  misc_feature 1..4244: note = Synthetic: Halo construct comprising Zero-CpG a cruciformstructure variant 1, and human TBG liver-specific promoter-driven Zero-CpG human sTNALP-Fc-(DSS)6 transgene
  source 1..4244: mol_type = other DNA
ctaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggtttcctttaattaagcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtccctgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaatttaaataggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtgctagcccaccatggatatgagagtacctgctcaacttctgggtttgttgctgctttggctcaggggagccagatgtgctgccctggttcctgagaaggaaaaggaccccaagtactggagagatcaggcacaggagacactgaagtatgccctggagctgcagaagctgaataccaatgttgccaaaaatgtgatcatgttcctgggagatgggatgggagtcagcacagtgacagctgctagaatcctgaagggacagctgcatcataatcctggagaagagaccagactggaaatggataagttcccctttgtggccctgagcaagacatataataccaatgcccaagtgcctgattctgctggaacagctacagcttacctgtgtggagtgaaggccaatgagggaacagtgggagtgtctgctgccacagaaagaagcagatgcaacaccacccaaggcaatgaggtgacaagcatcctgagatgggccaaggatgctggcaaatctgtgggcattgtgacaacaaccagagtgaaccatgccaccccatctgctgcttatgcccattctgctgacagagactggtactctgacaatgagatgcctccagaggctctgtcccagggttgtaaagatattgcctatcagctgatgcacaacatcagagacattgatgtgatcatgggaggaggcaggaagtacatgtaccccaagaacaagacagatgtggagtatgagtctgatgagaaggccagaggcacaagactggatggcctggacctggtggacacctggaagtcttttaagcccagatacaagcacagccacttcatctggaacagaacagaactgctgaccctggacccccacaatgtggactacctgctgggcctgtttgagcctggagatatgcagtatgagctgaatagaaacaatgtgacagacccctccctgtctgaaatggtggtggtggccatccagatcctgagaaagaaccctaagggctttttcctgctggttgagggaggaagaattgaccatggccatcatgagggcaaggctaagcaggccctgcatgaggctgtggagatggacagagccattggccaagctggctctctgaccagctcagaggacaccctgacagtggtgacagctgatcactctcatgtgttcacttttggaggatatacccctagaggcaacagcatctttggcctggcccctatgctgtctgacacagacaaaaagcccttcacagccatcctgtatggcaatggcccaggatacaaggtggtgggaggagagagagagaatgtgtctatggtggactatgcacacaacaactaccaggcccagtctgctgttcctctgagacatgaaacacatggaggggaagatgtggcagtgttttctaaaggccctatggctcacctgctgcatggagtccatgagcagaactatgtgccccatgtgatggcctatgctgcctgcattggagcaaacctgggccactgtgcccctgccagcagcctgaaagacaagacccacacatgtcctccctgccctgcccctgagctgctgggaggaccttctgtgttcctgttccctccaaagcctaaagacaccctgatgatcagcagaacccctgaagtgacttgtgtggtggtggatgtcagccatgaagatccagaagtgaaattcaactggtatgtggatggagtggaggtgcacaatgccaaaaccaagcctagagaagaacagtacaacagcacctacagagtggtgtctgtgctgacagtgcttcaccaggactggctgaatggcaaggaatacaagtgcaaggtgagcaacaaggccctgcctgctcctattgagaagaccatcagcaaggccaagggacagcccagagaacctcaggtctacaccctgcctcctagcagagatgagctgacaaagaaccaggtgagcctgacatgcctggtgaagggcttctacccttctgatattgctgtggaatgggagtctaatggccaacctgaaaacaactacaagacaacaccccctgtgctggactctgatggcagcttcttcctgtactccaaactgacagtggacaagtccagatggcagcagggcaatgtgttcagttgctctgtcatgcatgaagccctgcacaaccactacacccagaaaagcctcagcctgagccctggaaaggatattgactcctctgattcttctgacagctcagactcctctgatagctctgacagctcttgaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggataccgttcgtataatgtatgctatacgaacggtactgcagtgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcttaattaagg

SEQ ID NO: 49 - DNA, 186 bp, synthetic construct
  misc_feature 1..186: note = Synthetic: Zero-CpG A cruciform structure 1
  source 1..186: mol_type = other DNA
ttaattaaggctaggaacccctagtgatggagttggccactccctctctgccctctccctctctcactgaggcctggggaccaaaggtccccagactgcagagctttgctctgcaggcctcagtgagagagggagagggcagagagggagtggccaactccatcactaggggtttcctttaattaa

SEQ ID NO: 50 - DNA, 166 bp, synthetic construct
  misc_feature 1..166: note = Synthetic: Zero-CpG A cruciform structure 2
  source 1..166: mol_type = other DNA
aggaacccctagtgatggagttggccactccctctctgcactctctctctctcactgaggcctggggaccaaaggtccccagactgcagagctttgctctgcaggcctcagtgagagagagagagtgcagagagggagtggccaactccatcactaggggtttcct

SEQ ID NO: 51 - DNA, 166 bp, synthetic construct
  misc_feature 1..166: note = Synthetic: Zero-CpG A cruciform structure 3
  source 1..166: mol_type = other DNA
aggaacccctagtgatggagttggccactccctctctgctcactcactcactcactgaggccagggcaccaaaggtgccctgacagccctgctttgcagggctggcctcagtgagtgagtgagtgagcagagagggagtggccaactccatcactaggggtttcct

SEQ ID NO: 52 - DNA, 2349 bp, synthetic construct
  misc_feature 1..2349: note = Synthetic: Zero-CpG human soluble TNALP-Fc-(DSS)6
  source 1..2349: mol_type = other DNA
atggatatgagagtacctgctcaacttctgggtttgttgctgctttggctcaggggagccagatgtgctgccctggttcctgagaaggaaaaggaccccaagtactggagagatcaggcacaggagacactgaagtatgccctggagctgcagaagctgaataccaatgttgccaaaaatgtgatcatgttcctgggagatgggatgggagtcagcacagtgacagctgctagaatcctgaagggacagctgcatcataatcctggagaagagaccagactggaaatggataagttcccctttgtggccctgagcaagacatataataccaatgcccaagtgcctgattctgctggaacagctacagcttacctgtgtggagtgaaggccaatgagggaacagtgggagtgtctgctgccacagaaagaagcagatgcaacaccacccaaggcaatgaggtgacaagcatcctgagatgggccaaggatgctggcaaatctgtgggcattgtgacaacaaccagagtgaaccatgccaccccatctgctgcttatgcccattctgctgacagagactggtactctgacaatgagatgcctccagaggctctgtcccagggttgtaaagatattgcctatcagctgatgcacaacatcagagacattgatgtgatcatgggaggaggcaggaagtacatgtaccccaagaacaagacagatgtggagtatgagtctgatgagaaggccagaggcacaagactggatggcctggacctggtggacacctggaagtcttttaagcccagatacaagcacagccacttcatctggaacagaacagaactgctgaccctggacccccacaatgtggactacctgctgggcctgtttgagcctggagatatgcagtatgagctgaatagaaacaatgtgacagacccctccctgtctgaaatggtggtggtggccatccagatcctgagaaagaaccctaagggctttttcctgctggttgagggaggaagaattgaccatggccatcatgagggcaaggctaagcaggccctgcatgaggctgtggagatggacagagccattggccaagctggctctctgaccagctcagaggacaccctgacagtggtgacagctgatcactctcatgtgttcacttttggaggatatacccctagaggcaacagcatctttggcctggcccctatgctgtctgacacagacaaaaagcccttcacagccatcctgtatggcaatggcccaggatacaaggtggtgggaggagagagagagaatgtgtctatggtggactatgcacacaacaactaccaggcccagtctgctgttcctctgagacatgaaacacatggaggggaagatgtggcagtgttttctaaaggccctatggctcacctgctgcatggagtccatgagcagaactatgtgccccatgtgatggcctatgctgcctgcattggagcaaacctgggccactgtgcccctgccagcagcctgaaagacaagacccacacatgtcctccctgccctgcccctgagctgctgggaggaccttctgtgttcctgttccctccaaagcctaaagacaccctgatgatcagcagaacccctgaagtgacttgtgtggtggtggatgtcagccatgaagatccagaagtgaaattcaactggtatgtggatggagtggaggtgcacaatgccaaaaccaagcctagagaagaacagtacaacagcacctacagagtggtgtctgtgctgacagtgcttcaccaggactggctgaatggcaaggaatacaagtgcaaggtgagcaacaaggccctgcctgctcctattgagaagaccatcagcaaggccaagggacagcccagagaacctcaggtctacaccctgcctcctagcagagatgagctgacaaagaaccaggtgagcctgacatgcctggtgaagggcttctacccttctgatattgctgtggaatgggagtctaatggccaacctgaaaacaactacaagacaacaccccctgtgctggactctgatggcagcttcttcctgtactccaaactgacagtggacaagtccagatggcagcagggcaatgtgttcagttgctctgtcatgcatgaagccctgcacaaccactacacccagaaaagcctcagcctgagccctggaaaggatattgactcctctgattcttctgacagctcagactcctctgatagctctgacagctcttgaactgtgccttctagttgccagccatctgttgtttgcccctcccccgtgccttccttgaccctggaaggtgcc

SEQ ID NO: 53 - DNA, 2079 bp, synthetic construct
  misc_feature 1..2079: note = Synthetic: Zero-CpG human PCSK9 Q162H
  source 1..2079: mol_type = other DNA
atgggcacagtgagctccagaagatcttggtggcctctgcctctgctgctgctcctgctgctgctgctgggacctgctggagctagagcccaggaggatgaagatggagactatgaggagctggtgctggctctgagatctgaggaagatggtctggctgaagcccctgagcatggcaccacagctacctttcacagatgtgccaaggacccctggagactgcctggcacctatgtggtggttcttaaagaggagacacatctgagccagtctgagagaacagccagaagactgcaggcccaagctgctagaagaggttatctgaccaagatcctgcatgtgtttcatggccttcttcctggcttcctggtcaagatgtctggagatctgcttgagctggccctgaagctgccccatgtggattacattgaggaggatagctctgtgtttgcccactctattccttggaacctggaaagaatcacccctccaagatatagagctgatgaataccagcctcctgatggaggcagcctggtggaggtgtacctgctggacaccagcatccagtctgaccacagggagattgagggcagagttatggtgacagattttgagaatgtgccagaagaggatggcaccagattccacagacaagccagcaagtgtgacagccatggcacccacctggctggagtggtgtctggcagagatgctggagtggccaagggagccagcatgagaagcctgagagtgctgaattgccagggcaagggcacagtgtctggcaccctaattggactggagttcatcagaaagagccagctggtgcagcctgtgggacctctggttgttctgctgcctctagctggaggatacagcagagtgctgaatgctgcctgccagagactggctagagctggagtggtgctggtgactgctgctggaaatttcagagatgatgcctgcctgtacagccctgcctctgcccctgaagtgatcacagtgggagctacaaatgcccaggaccaaccagtgaccctgggcaccctgggaaccaactttggcagatgtgtggacctgtttgcccctggagaggacatcattggagccagctcagattgctctacatgctttgtgagccaatctggcacctcacaggctgctgctcatgtggctggcattgctgccatgatgctgtctgcagaacctgagctgacactggctgagctgagacagagactgatccacttctctgctaaagatgtgatcaatgaggcctggttccctgaagaccagagagtgctgacacctaatctggtggctgctctgcctcccagcacccatggagctggatggcagctgttctgcagaacagtgtggtctgcccactctggccccaccagaatggccacagctgtggccagatgtgcccctgatgaggaactgctcagctgcagcagctttagcagatctggcaaaagaagaggagaaagaatggaagcccagggaggcaagctggtgtgcagagcccacaatgcctttggaggagaaggagtgtatgccattgccagatgttgcctgctgccccaggccaactgctctgtgcacacagcccctccagctgaggcctctatgggcaccagagtgcactgccaccagcagggccatgtgctgacaggctgttctagccattgggaggtggaagacctgggaacccacaagcctcctgtgctgagacccagaggccagcctaaccagtgtgtgggccacagagaagccagcatccatgccagttgttgtcatgcccctggcctggaatgcaaggtgaaggaacatggcatcccagctccacaggagcaggtgacagtggcctgtgaggaaggctggaccctgacaggctgctctgccctgcctggcacaagccatgtgctgggagcatatgctgtggacaacacctgtgtggtgagaagcagggatgtgagcacaacaggcagcacctctgagggagctgttacagctgtggccatctgctgtagatctagacacttggcccaggcctctcaggagctgcagtga

SEQ ID NO: 54 - DNA, 1524 bp, synthetic construct
  misc_feature 1..1524: note = Synthetic: Zero-CpG cynomolgus monkey soluble TNALP
  source 1..1524: mol_type = other DNA
atgagagtgcctgcccagctgctgggcctgctgctgctgtggctgagaggagccagatgtgctgccctggtgcctgagaaggagaaggaccccaagtactggagagaccaggcccaggagaccctgaagtatgccctggagctgcagaagctgaacaccaatgtggccaagaatgtgatcatgttcctgggagatggcatgggagtgagcacagtgacagccaccagaatcctgaagggccagctgcaccacaaccctggagaggagaccagactggagatggacaagttcccctttgtggccctgagcaagacctacaacaccaatgcccaggtgcctgactctgctggcacagccacagcctacctgtgtggagtgaaggccaatgagggcacagtgggagtgtctgctgccacagagagaagcagatgcaacaccacccagggcaatgaggtgaccagcatcctgagatgggccaaggatgctggcaagtctgtgggcattgtgaccaccaccagagtgaaccatgccaccccctctgctgcctatgcccactctgctgacagagactggtactctgacaatgagatgccccctgaggccctgagccagggctgcaaggacattgcctaccagctggtgcacaacatcagagacattgatgtgatcatgggaggaggcagaaagtacatgtaccccaagaacaagacagatgtggagtatgagattgatgagaaggccagaggcaccagactggatggcctggacctggtgaacatctggaagagcttcaagcccagacacaagcacagccacttcatctggaacagaacagagctgctgaccctggacccccacaatgtggactacctgctgggcctgtttgagcctggagacatggagtatgagctgaacagaaacaatgtgacagaccccagcctgtctgagatggtggtggtggccatccagatcctgagaaagaaccccaagggcttcttcctgctggtggagggaggcagaattgaccatggccaccatgagggcaaggccaagcaggccctgcatgaggctgtggagatggacagagccattggccaggctggcagcatgaccagcctggaggacaccctgacagtggtgacagctgaccacagccatgtgttcacctttggaggctacacccccagaggcaacagcatctttggcctggcccccatgctgtctgacacagacaagaagcccttcacagccatcctgtatggcaatggccctggctacaaggtggtgggaggagagagagagaatgtgagcatggtggactatgcccacaacaactaccaggcccagtctgctgtgcccctgagacatgagacccatggaggagaggatgtggctgtgttcagcaagggccccatggcccacctgctgcatggagtgcatgagcagaactacatcccccatgtgatggcctatgctgcctgcattggagccaacctggaccactgtgcccctgccagcagctga

SEQ ID NO: 55 - DNA, 7360 bp, synthetic construct
  misc_feature 1..7360: note = Synthetic: P004 - a BC-deletion cruciform structure-contained A dsGFP-dsRFP reporter construct
  source 1..7360: mol_type = other DNA
acgcgttgacattgattattgactagttaactataacggtcctaaggtagcgaacccccaactgagagaactcaaaggttaccccagttggggtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattcgaagcttgagctcgagatcctactagtaggatctgagtccggattacttgtacagctcgtccatgccgtgagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccaggaacccctagtgatggagttggccactccctctctgcgcctcgctcgctcactgaggccgccccgggcctcagtgagcgagcgagcgcgcagagagggagtggccaactccatcactaggggtttcctctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtagcgtgctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccgagctcggtaccacatgtaagcttcgaggggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcttaattaaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcgccaccatggatagcactgagaacgtcatcaagcccttcatgcgcttcaaggtgcacatggagggctccgtgaacggccacgagttcgagatcgagggcgagggcgagggcaagccctacgagggcacccagaccgccaagctgcaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggcaccttcatctaccacgtgaagttcatcggcgtgaacttcccctccgacggccccgtaatgcagaagaagactctgggctgggagccctccaccgagcgcctgtacccccgcgacggcgtgctgaagggcgagatccacaaggcgctgaagctgaagggcggcggccactacctggtggagttcaagtcaatctacatggccaagaagcccgtgaagctgcccggctactactacgtggactccaagctggacatcacctcccacaacgaggactacaccgtggtggagcagtacgagcgcgccgaggcccgccaccacctgttccagtgagaattcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctccgcgcccggggagcccaagggcacgccctggcacctagggggtatccccgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatat

SEQ ID NO: 56 - DNA, 7956 bp, synthetic construct
  misc_feature 1..7956: note = Synthetic: minimal R6K Ori sequence between two inverted repeat sequences contained dsGFP-dsRFP reporter construct
  source 1..7956: mol_type = other DNA
acgcgttgacattgattattgactagttaactataacggtcctaaggtagcgaacccccaactgagagaactcaaaggttaccccagttggggtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattcgaagcttgagctcgagatcctactagtaggatctgagtccggattacttgtacagctcgtccatgccgtgagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctgcaggaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaataccgttcgtataatgtatgctatacgaacggtactgcagtgtcagccgttaagtgttcctgtgtcactcaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttcctttcttacctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtagcgtgctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccgagctcggtaccacatgtaagcttcgaggggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcttaattaaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcgccaccatggatagcactgagaacgtcatcaagcccttcatgcgcttcaaggtgcacatggagggctccgtgaacggccacgagttcgagatcgagggcgagggcgagggcaagccctacgagggcacccagaccgccaagctgcaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggcaccttcatctaccacgtgaagttcatcggcgtgaacttcccctccgacggccccgtaatgcagaagaagactctgggctgggagccctccaccgagcgcctgtacccccgcgacggcgtgctgaagggcgagatccacaaggcgctgaagctgaagggcggcggccactacctggtggagttcaagtcaatctacatggccaagaagcccgtgaagctgcccggctactactacgtggactccaagctggacatcacctcccacaacgaggactacaccgtggtggagcagtacgagcgcgccgaggcccgccaccacctgttccagtgagaattcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctccgcgcccggggagcccaagggcacgccctggcacctagggggtatccccgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatat

SEQ ID NO: 57 - DNA, 7379 bp, synthetic construct
  misc_feature 1..7379: note = Synthetic: A BC-deleted & RBS-mutated A cruciform structure contained dsGFP-dsRFP reporter construct
  source 1..7379: mol_type = other DNA
acgcgttgacattgattattgactagttaactataacggtcctaaggtagcgaacccccaactgagagaactcaaaggttaccccagttggggtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattcgaagcttgagctcgagatcctactagtaggatctgagtccggattacttgtacagctcgtccatgccgtgagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctgcaggaggaggaacccctagtgatggggccggccactccctctctcctccctccctccctcactgaggccgccccgcggcctcagtgagcgagcgagcgcgcagagagggagtggccaactccatcactaggggtttcctttcttacctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtagcgtgctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccctgaagttctcaggatccgagctcggtaccacatgtaagcttcgaggggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcttaattaaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcgccaccatggatagcactgagaacgtcatcaagcccttcatgcgcttcaaggtgcacatggagggctccgtgaacggccacgagttcgagatcgagggcgagggcgagggcaagccctacgagggcacccagaccgccaagctgcaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggcaccttcatctaccacgtgaagttcatcggcgtgaacttcccctccgacggccccgtaatgcagaagaagactctgggctgggagccctccaccgagcgcctgtacccccgcgacggcgtgctgaagggcgagatccacaaggcgctgaagctgaagggcggcggccactacctggtggagttcaagtcaatctacatggccaagaagcccgtgaagctgcccggctactactacgtggactccaagctggacatcacctcccacaacgaggactacaccgtggtggagcagtacgagcgcgccgaggcccgccaccacctgttccagtgagaattcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctccgcgcccggggagcccaagggcacgccctggcacctagggggtatccccgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatat

SEQ ID NO: 58 - DNA, 303 bp, synthetic construct
  misc_feature 1..303: note = Synthetic: Wildtype R6K gamma origin
  source 1..303: mol_type = other DNA
ggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatc

SEQ ID NO: 59 - DNA, 250 bp, synthetic construct
  misc_feature 1..250: note = Synthetic: Zero-CpG R6K gamma origin
  source 1..250: mol_type = other DNA
aaaccttaaaacctttaaaagccttatatattcttttttttcttataaaacttaaaaccttagaggctatttaagttgctgatttatattaattttattgttcaaacatgagagcttagtacatgaaacatgagagcttagtacattagccatgagagcttagtacattagccatgagggtttagttcattaaacatgagagcttagtacaacaggttgaattaaacatgagagcttagtacatactatc

SEQ ID NO: 60 - DNA, 656 bp, synthetic construct
  misc_feature 1..656: note = Synthetic: Two AAV2 inverted repeat sequences separated by a Zero-CpG R6K gamma origin of replication (Ori) sequence
  source 1..656: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaataccgttcgtataatgtatgctatacgaacggtactgcagtgtcagccgttaagtgttcctgtgtcactcaaaattgctttgagaggctctaagggcttctcagtgcgttacatccctaaaccttaaaacctttaaaagccttatatattcttttttttcttataaaacttaaaaccttagaggctatttaagttgctgatttatattaattttattgttcaaacatgagagcttagtacatgaaacatgagagcttagtacattagccatgagagcttagtacattagccatgagggtttagttcattaaacatgagagcttagtacaacaggttgaattaaacatgagagcttagtacatactatcttcagatcttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 61 - DNA, 995 bp, synthetic construct
  misc_feature 1..995: note = Synthetic: Two AAV2 inverted repeat sequences separated by a pUc origin of replication (Ori) sequence
  source 1..995: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaataccgttcgtataatgtatgctatacgaacggtactgcagtgtcagccgttaagtgttcctgtgtcactcaaaattgctttgagaggctctaagggcttctcagtgcgttacatcccttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaattcagatcttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 62 - DNA, 657 bp, synthetic construct
  misc_feature 1..657: note = Synthetic: Two Zero-CpG AAV2 inverted repeat sequences separated by a Zero-CpG R6K gamma origin of replication sequence
  source 1..657: mol_type = other DNA
aggaacccctagtgatggagttggccactccctctctgcactctctctctctcactgaggcctggggaccaaaggtccccagactgcagagctttgctctgcaggcctcagtgagagagagagagtgcagagccttaattaataccgttcgtataatgtatgctatacgaacggtactgcagtgtcagccgttaagtgttcctgtgtcactcaaaattgctttgagaggctctaagggcttctcagtgcgttacatccctaaaccttaaaacctttaaaagccttatatattcttttttttcttataaaacttaaaaccttagaggctatttaagttgctgatttatattaattttattgttcaaacatgagagcttagtacatgaaacatgagagcttagtacattagccatgagagcttagtacattagccatgagggtttagttcattaaacatgagagcttagtacaacaggttgaattaaacatgagagcttagtacatactatcttcagatcttaattaaggctctcactgaggcctgcagagcaaagctctgcagtctggggacctttggtccccaggcctcagtgagagagggagagggcagagagggagtggccaactccatcactaggggttcctagccttaattaa

SEQ ID NO: 63 - DNA, 1524 bp, synthetic construct
  misc_feature 1..1524: note = Synthetic: Zero-CpG murine secreted embryonic alkaline phosphatase (SEAP)
  source 1..1524: mol_type = other DNA
atgtggggagcctgcctgctgctgctgggcctgagcctgcaggtgtgcccctctgtgatccctgtggaggaggagaaccctgccttctggaacagaaaggctgctgaggccctggatgctgccaagaagctgaagcccatccagacctctgccaagaacctggtgatcctgatgggagatggcatgggagtgagcacagtgacagccaccagaatcctgaagggccagcagcagggccacctgggccctgagacccagctggccatggacagattcccccacatggccctgagcaagacctacaacacagacaagcagatccctgactctgctggcacaggcacagccttcctgtgtggagtgaagaccaacatgaaggtgattggcctgtctgctgctgccagattcaaccagtgcaacaccacctggggcaatgaggtggtgtctgtgatgcacagagccaagaaggctggcaagtctgtgggagtggtgaccaccacctctgtgcagcatgccagccctgctggcacctatgcccacacagtgaacagaggctggtactctgatgcccagatgcctgcctctgccctgcaggatggctgcaaggacatcagcacccagctgatcagcaacatggacattgatgtgatcctgggaggaggcagaaagttcatgttccccaagggcacccctgaccaggagtaccccacagacaccaagcaggctggcaccagactggatggcagaaacctggtgcaggagtggctggccaagcaccagggagccagatatgtgtggaacagatctgagctgatccaggccagcctgaacagatctgtgacccacctgatgggcctgtttgagcccaatgacatgaagtatgagatccacagagaccctgcccaggaccccagcctggctgagatgacagaggtggctgtgagaatgctgagcagaaaccccaagggcttctacctgtttgtggagggaggcagaattgaccatggccaccatgagacagtggcctacagagccctgacagaggctgtgatgtttgactctgctgtggacaaggctgacaagctgacctctgagcaggacaccatgatcctggtgacagctgaccacagccatgtgttcagctttggaggctacacccagagaggagccagcatctttggcctggcccccttcaaggctgaggatggcaagagcttcaccagcatcctgtatggcaatggccctggctacaagctgcacaatggagccagagctgatgtgacagaggaggagagcagcaaccccacctaccagcagcaggctgctgtgcccctgagctctgagacccactctggagaggatgtggccatctttgccagaggcccccaggcccacctggtgcatggagtgcaggagcagaactacattgcccatgtgatggcctttgctgcctgcctggagccctacacagactgtggcctggccagccctgctggccagagctctgctgtgagccctggctga

SEQ ID NO: 64 - protein, 1279 aa, Homo sapiens
  REGION 1..1279: note = misc_feature - Amino acid sequence of ABCB4
MDLEAAKNGTAWRPTSAEGDFELGISSKQKRKKTKTVKMIGVLTLFRYSDWQDKLFMSLGTIMAIAHGSGLPLMMIVFGEMTDKFVDTAGNFSFPVNFSLSLLNPGKILEEEMTRYAYYYSGLGAGVLVAAYIQVSFWTLAAGRQIRKIRQKFFHAILRQEIGWFDINDTTELNTRLTDDISKISEGIGDKVGMFFQAVATFFAGFIVGFIRGWKLTLVIMAISPILGLSAAVWAKILSAFSDKELAAYAKAGAVAEEALGAIRTVIAFGGQNKELERYQKHLENAKEIGIKKAISANISMGIAFLLIYASYALAFWYGSTLVISKEYTIGNAMTVFFSILIGAFSVGQAAPCIDAFANARGAAYVIFDIIDNNPKIDSFSERGHKPDSIKGNLEFNDVHFSYPSRANVKILKGLNLKVQSGQTVALVGSSGCGKSTTVQLIQRLYDPDEGTINIDGQDIRNFNVNYLREIIGVVSQEPVLFSTTIAENICYGRGNVTMDEIKKAVKEANAYEFIMKLPQKFDTLVGERGAQLSGGQKQRIAIARALVRNPKILLLDEATSALDTESEAEVQAALDKAREGRTTIVIAHRLSTVRNADVIAGFEDGVIVEQGSHSELMKKEGVYFKLVNMQTSGSQIQSEEFELNDEKAATRMAPNGWKSRLFRHSTQKNLKNSQMCQKSLDVETDGLEANVPPVSFLKVLKLNKTEWPYFVVGTVCAIANGGLQPAFSVIFSEIIAIFGPGDDAVKQQKCNIFSLIFLFLGIISFFTFFLQGFTFGKAGEILTRRLRSMAFKAMLRQDMSWFDDHKNSTGALSTRLATDAAQVQGATGTRLALIAQNIANLGTGIIISFIYGWQLTLLLLAVVPIIAVSGIVEMKLLAGNAKRDKKELEAAGKIATEAIENIRTVVSLTQERKFESMYVEKLYGPYRNSVQKAHIYGITFSISQAFMYFSYAGCFRFGAYLIVNGHMRFRDVILVFSAIVFGAVALGHASSFAPDYAKAKLSAAHLFMLFERQPLIDSYSEEGLKPDKFEGNITFNEVVFNYPTRANVPVLQGLSLEVKKGQTLALVGSSGCGKSTVVQLLERFYDPLAGTVLLDGQEAKKLNVQWLRAQLGIVSQEPILFDCSIAENIAYGDNSRVVSQDEIVSAAKAANIHPFIETLPHKYETRVGDKGTQLSGGQKQRIAIARALIRQPQILLLDEATSALDTESEKVVQEALDKAREGRTCIVIAHRLSTIQNADLIVVFQNGRVKEHGTHQQLLAQKGIYFSMVSVQAGTQNL

SEQ ID NO: 65 - DNA, 3840 bp, synthetic construct
  misc_feature 1..3840: note = Synthetic: Codon-optimized ABCB4 sequence
  source 1..3840: mol_type = other DNA
atggatctggaggccgccaagaatggcaccgcatggagacccacaagcgccgagggcgacttcgagctgggcatcagctccaagcagaagagaaagaagaccaagacagtgaagatgatcggcgtgctgacactgttcaggtactccgactggcaggataagctgtttatgtctctgggcaccatcatggcaatcgcccacggcagcggactgcctctgatgatgatcgtgttcggcgagatgaccgacaagtttgtggatacagccggcaatttctcctttcccgtgaacttctctctgagcctgctgaaccctggcaagatcctggaggaggagatgacaagatatgcctactattactctggactgggagcaggcgtgctggtggcagcatacatccaggtgagcttctggaccctggccgcaggccggcagatcagaaagatcaggcagaagttctttcacgccatcctgcgccaggagatcggctggtttgacatcaatgataccacagagctgaacacccggctgacagacgacatctctaagatcagcgagggcatcggcgataaagtgggcatgttctttcaggccgtggccacattctttgccggcttcatcgtgggctttatcaggggctggaagctgaccctggtcatcatggccatctctccaatcctgggactgagcgccgccgtgtgggcaaagatcctgtccgccttctctgacaaggagctggccgcctacgccaaggcaggagcagtggcagaggaggccctgggcgccatccgcaccgtgatcgcctttggcggccagaataaggagctggagcggtatcagaagcacctggagaacgccaaggagatcggcatcaagaaggccatctccgccaatatctctatgggcatcgccttcctgctgatctatgccagctacgccctggccttttggtatggcagcaccctggtcatcagcaaggagtacaccatcggcaatgccatgacagtgttctttagcatcctgatcggagccttctccgtgggacaggcagcaccatgcatcgacgccttcgccaacgccagaggcgcagcctacgtgatctttgacatcatcgataacaatccaaagatcgactccttttctgagcggggccacaagcccgattccatcaagggcaatctggagttcaacgacgtgcactttagctatccctcccgcgccaatgtgaagatcctgaagggcctgaacctgaaggtgcagtccggacagacagtggccctggtgggctctagcggatgcggcaagtctaccacagtgcagctgatccagcggctgtatgacccagatgagggcaccatcaacatcgacggccaggacatccgcaacttcaatgtgaactacctgcgggagatcatcggcgtggtgtctcaggagcccgtgctgtttagcaccacaatcgccgagaatatctgttatggccgcggcaacgtgacaatggatgagatcaagaaggccgtgaaggaggccaatgcctacgagttcatcatgaagctgccccagaagtttgacaccctggtgggagagagaggcgcccagctgagcggaggccagaagcagaggatcgccatcgccagagccctggtgaggaaccctaagatcctgctgctggacgaggccacctccgccctggatacagagtctgaggccgaggtgcaggccgccctggataaggcccgcgagggacggaccacaatcgtgatcgcccacagactgagcacagtgaggaatgccgacgtgatcgccggcttcgaggatggcgtgatcgtggagcagggcagccactccgagctgatgaagaaggagggcgtgtacttcaagctggtgaacatgcagacctctggcagccagatccagtccgaggagtttgagctgaatgacgagaaggcagcaacaaggatggcacctaacggatggaagagcagactgttcaggcactccacccagaagaatctgaagaactctcagatgtgccagaagagcctggacgtggagaccgatggcctggaggcaaatgtgccacccgtgagcttcctgaaggtgctgaagctgaacaagacagagtggccatattttgtggtgggcaccgtgtgcgcaatcgcaaatggaggactgcagccagccttcagcgtgatcttttccgagatcatcgccatcttcggccctggcgacgatgccgtgaagcagcagaagtgtaacatcttttccctgatcttcctgtttctgggcatcatctctttctttaccttctttctgcagggcttcacatttggcaaggccggcgagatcctgacacggagactgagaagcatggccttcaaggccatgctgaggcaggatatgtcctggtttgacgatcacaagaacagcaccggcgccctgtccaccagactggccacagacgcagcacaggtgcagggagcaaccggcacaaggctggccctgatcgcccagaatatcgccaacctgggcacaggcatcatcatctctttcatctacggatggcagctgaccctgctgctgctggccgtggtgccaatcatcgccgtgagcggcatcgtggagatgaagctgctggccggcaatgccaagagagacaagaaggagctggaggcagcaggcaagatcgcaaccgaggccatcgagaacatccgcaccgtggtgagcctgacacaggagcggaagttcgagtccatgtatgtggagaagctgtatggcccctaccgcaattccgtgcagaaggcccacatctacggcatcaccttttccatctctcaggccttcatgtatttttcttacgccggctgcttccggtttggcgcctatctgatcgtgaacggccacatgaggttccgcgatgtgatcctggtgttctctgccatcgtgtttggagcagtggccctgggacacgcctcctcttttgcccctgactatgcaaaggcaaagctgtccgccgcacacctgttcatgctgtttgagagacagcctctgatcgatagctactccgaggagggcctgaagccagacaagttcgagggcaatatcacattcaacgaggtggtgtttaattatccaaccagggccaacgtgcccgtgctgcagggactgagcctggaggtgaagaagggacagacactggccctggtgggcagctccggatgcggcaagtccaccgtggtgcagctgctggagagattctacgaccctctggccggcaccgtgctgctggatggacaggaggccaagaagctgaatgtgcagtggctgagggcccagctgggcatcgtgtctcaggagccaatcctgttcgattgtagcatcgccgagaatatcgcctacggcgacaactctagggtggtgagccaggatgagatcgtgagcgccgcaaaggcagcaaacatccacccttttatcgagaccctgccacacaagtatgagacacgcgtgggcgacaagggcacccagctgtccggaggacagaagcagaggatcgcaatcgcccgcgccctgatcaggcagccccaaatcctgctgctggacgaggccacctccgccctggataccgagtctgagaaggtggtgcaggaggccctggataaagcccgcgagggacggacatgtattgtcattgctcacagactgagcaccatccagaatgccgacctgatcgtggtgttccagaacggcagggtgaaggagcacggcacacaccagcagctgctggcccagaagggaatctacttttctatggtgagcgtgcaggccggcacccagaacctgtga

SEQ ID NO: 66 - DNA, 3840 bp, Homo sapiens
  misc_feature 1..3840: note = ABCB4 cDNA sequence
  source 1..3840: mol_type = genomic DNA
atggatcttgaggcggcaaagaacggaacagcctggcgccccacgagcgcggagggcgactttgaactgggcatcagcagcaaacaaaaaaggaaaaaaacgaagacagtgaaaatgattggagtattaacattgtttcgatactccgattggcaggataaattgtttatgtcgctgggtaccatcatggccatagctcacggatcaggtctccccctcatgatgatagtatttggagagatgactgacaaatttgttgatactgcaggaaacttctcctttccagtgaacttttccttgtcgctgctaaatccaggcaaaattctggaagaagaaatgactagatatgcatattactactcaggattgggtgctggagttcttgttgctgcctatatacaagtttcattttggactttggcagctggtcgacagatcaggaaaattaggcagaagttttttcatgctattctacgacaggaaataggatggtttgacatcaacgacaccactgaactcaatacgcggctaacagatgacatctccaaaatcagtgaaggaattggtgacaaggttggaatgttctttcaagcagtagccacgttttttgcaggattcatagtgggattcatcagaggatggaagctcacccttgtgataatggccatcagccctattctaggactctctgcagccgtttgggcaaagatactctcggcatttagtgacaaagaactagctgcttatgcaaaagcaggcgccgtggcagaagaggctctgggggccatcaggactgtgatagctttcgggggccagaacaaagagctggaaaggtatcagaaacatttagaaaatgccaaagagattggaattaaaaaagctatttcagcaaacatttccatgggtattgccttcctgttaatatatgcatcatatgcactggccttctggtatggatccactctagtcatatcaaaagaatatactattggaaatgcaatgacagtttttttttcaatcctaattggagctttcagtgttggccaggctgccccatgtattgatgcttttgccaatgcaagaggagcagcatatgtgatctttgatattattgataataatcctaaaattgacagtttttcagagagaggacacaaaccagacagcatcaaagggaatttggagttcaatgatgttcacttttcttacccttctcgagctaacgtcaagatcttgaagggcctcaacctgaaggtgcagagtgggcagacggtggccctggttggaagtagtggctgtgggaagagcacaacggtccagctgatacagaggctctatgaccctgatgagggcacaattaacattgatgggcaggatattaggaactttaatgtaaactatctgagggaaatcattggtgtggtgagtcaggagccggtgctgttttccaccacaattgctgaaaatatttgttatggccgtggaaatgtaaccatggatgagataaagaaagctgtcaaagaggccaacgcctatgagtttatcatgaaattaccacagaaatttgacaccctggttggagagagaggggcccagctgagtggtgggcagaagcagaggatcgccattgcacgtgccctggttcgcaaccccaagatccttctgctggatgaggccacgtcagcattggacacagaaagtgaagctgaggtacaggcagctctggataaggccagagaaggccggaccaccattgtgatagcacaccgactgtctacggtccgaaatgcagatgtcatcgctgggtttgaggatggagtaattgtggagcaaggaagccacagcgaactgatgaagaaggaaggggtgtacttcaaacttgtcaacatgcagacatcaggaagccagatccagtcagaagaatttgaactaaatgatgaaaaggctgccactagaatggccccaaatggctggaaatctcgcctatttaggcattctactcagaaaaaccttaaaaattcacaaatgtgtcagaagagccttgatgtggaaaccgatggacttgaagcaaatgtgccaccagtgtcctttctgaaggtcctgaaactgaataaaacagaatggccctactttgtcgtgggaacagtatgtgccattgccaatggggggcttcagccggcattttcagtcatattctcagagatcatagcgatttttggaccaggcgatgatgcagtgaagcagcagaagtgcaacatattctctttgattttcttatttctgggaattatttctttttttactttcttccttcagggtttcacgtttgggaaagctggcgagatcctcaccagaagactgcggtcaatggcttttaaagcaatgctaagacaggacatgagctggtttgatgaccataaaaacagtactggtgcactttctacaagacttgccacagatgctgcccaagtccaaggagccacaggaaccaggttggctttaattgcacagaatatagctaaccttggaactggtattatcatatcatttatctacggttggcagttaaccctattgctattagcagttgttccaattattgctgtgtcaggaattgttgaaatgaaattgttggctggaaatgccaaaagagataaaaaagaactggaagctgctggaaagattgcaacagaggcaatagaaaatattaggacagttgtgtctttgacccaggaaagaaaatttgaatcaatgtatgttgaaaaattgtatggaccttacaggaactctgtgcagaaggcacacatctatggaattacttttagtatctcacaagcatttatgtatttttcctatgccggttgttttcgatttggtgcatatctcattgtgaatggacatatgcgcttcagagatgttattctggtgttttctgcaattgtatttggtgcagtggctctaggacatgccagttcatttgctccagactatgctaaagctaagctgtctgcagcccacttattcatgctgtttgaaagacaacctctgattgacagctacagtgaagaggggctgaagcctgataaatttgaaggaaatataacatttaatgaagtcgtgttcaactatcccacccgagcaaacgtgccagtgcttcaggggctgagcctggaggtgaagaaaggccagacactagccctggtgggcagcagtggctgtgggaagagcacggtggtccagctcctggagcggttctacgaccccttggcggggacagtgcttctcgatggtcaagaagcaaagaaactcaatgtccagtggctcagagctcaactcggaatcgtgtctcaggagcctatcctatttgactgcagcattgccgagaatattgcctatggagacaacagccgggttgtatcacaggatgaaattgtgagtgcagccaaagctgccaacatacatcctttcatcgagacgttaccccacaaatatgaaacaagagtgggagataaggggactcagctctcaggaggtcaaaaacagaggattgctattgcccgagccctcatcagacaacctcaaatcctcctgttggatgaagctacatcagctctggatactgaaagtgaaaaggttgtccaagaagccctggacaaagccagagaaggccgcacctgcattgtgattgctcaccgcctgtccaccatccagaatgcagacttaatagtggtgtttcagaatgggagagtcaaggagcatggcacgcatcagcagctgctggcacagaaaggcatctatttttcaatggtcagtgtccaggctgggacacagaacttatga

SEQ ID NO: 67 - DNA, 1569 bp, synthetic construct
  misc_feature 1..1569: note = Synthetic: antiCD19-scFv-antiCD3-scFV sequence
  source 1..1569: mol_type = other DNA
atggatatgcgcgtacccgctcaacttctgggtttgttgctgctttggctcaggggagcccgctgtgccgcggacatccagctgacccagagccccgccagcctggccgtgagcctgggccagagagccaccatcagctgcaaggccagccagagcgtggactacgacggcgacagctacctgaactggtaccagcagatccccggccagccccccaagctgctgatctacgacgccagcaacctggtgagcggcatcccccccagattcagcggcagcggcagcggcaccgacttcaccctgaacatccaccccgtggagaaggtggacgccgccacctaccactgccagcagagcaccgaggacccctggaccttcggcggcggcaccaagctggagatcaagggcggcggcggcagcggcggcggcggcagcggcggcggcggcagccaggtgcagctgcagcagagcggcgccgagctggtgagacccggcagcagcgtgaagatcagctgcaaggccagcggctacgccttcagcagctactggatgaactgggtgaagcagagacccggccagggcctggagtggatcggccagatctggcccggcgacggcgacaccaactacaacggcaagttcaagggcaaggccaccctgaccgccgacgagagcagcagcaccgcctacatgcagctgagcagcctggccagcgaggacagcgccgtgtacttctgcgccagaagagagaccaccaccgtgggcagatactactacgccatggactactggggccagggcaccaccgtgaccgtgagcagcggcggcggcggcagcgacatcaagctgcagcagagcggcgccgagctggccagacccggcgccagcgtgaagatgagctgcaagaccagcggctacaccttcaccagatacaccatgcactgggtgaagcagagacccggccagggcctggagtggatcggctacatcaaccccagcagaggctacaccaactacaaccagaagttcaaggacaaggccaccctgaccaccgacaagagcagcagcaccgcctacatgcagctgagcagcctgaccagcgaggacagcgccgtgtactactgcgccagatactacgacgaccactactgcctggactactggggccagggcaccaccctgaccgtgagcagcgtggagggcggcagcggcggcagcggcggcagcggcggcagcggcggcgtggacgacatccagctgacccagagccccgccatcatgagcgccagccccggcgagaaggtgaccatgacctgcagagccagcagcagcgtgagctacatgaactggtaccagcagaagagcggcaccagccccaagagatggatctacgacaccagcaaggtggccagcggcgtgccctacagattcagcggcagcggcagcggcaccagctacagcctgaccatcagcagcatggaggccgaggacgccgccacctactactgccagcagtggagcagcaaccccctgaccttcggcgccggcaccaagctggagctgaagtag

SEQ ID NO: 68 - DNA, 4320 bp, synthetic construct
  misc_feature 1..4320: note = Synthetic: Zero-CpG B-domain-deleted Factor VIII sequence
  source 1..4320: mol_type = other DNA
atggccaccagaagatactacctgggagctgtggagctgagctgggactacatgcagtctgacctgggagagctgcctgtggatgccagattcccccccagagtgcccaagagcttccccttcaacacctctgtggtgtacaagaagaccctgtttgtggagttcacagaccacctgttcaacattgccaagcccagacccccctggatgggcctgctgggccccaccatccaggctgaggtgtatgacacagtggtgatcaccctgaagaacatggccagccaccctgtgagcctgcatgctgtgggagtgagctactggaaggcctctgagggagctgagtatgatgaccagaccagccagagagagaaggaggatgacaaggtgttccctggaggcagccacacctatgtgtggcaggtgctgaaggagaatggccccatggcctctgaccccctgtgcctgacctacagctacctgagccatgtggacctggtgaaggacctgaactctggcctgattggagccctgctggtgtgcagagagggcagcctggccaaggagaagacccagaccctgcacaagttcatcctgctgtttgctgtgtttgatgagggcaagagctggcactctgagaccaagaacagcctgatgcaggacagagatgctgcctctgccagagcctggcccaagatgcacacagtgaatggctatgtgaacagaagcctgcctggcctgattggctgccacagaaagtctgtgtactggcatgtgattggcatgggcaccacccctgaggtgcacagcatcttcctggagggccacaccttcctggtgagaaaccacagacaggccagcctggagatcagccccatcaccttcctgacagcccagaccctgctgatggacctgggccagttcctgctgttctgccacatcagcagccaccagcatgatggcatggaggcctatgtgaaggtggacagctgccctgaggagccccagctgagaatgaagaacaatgaggaggctgaggactatgatgatgacctgacagactctgagatggatgtggtgagatttgatgatgacaacagccccagcttcatccagatcagatctgtggccaagaagcaccccaagacctgggtgcactacattgctgctgaggaggaggactgggactatgcccccctggtgctggcccctgatgacagaagctacaagagccagtacctgaacaatggcccccagagaattggcagaaagtacaagaaggtgagattcatggcctacacagatgagaccttcaagaccagagaggccatccagcatgagtctggcatcctgggccccctgctgtatggagaggtgggagacaccctgctgatcatcttcaagaaccaggccagcagaccctacaacatctacccccatggcatcacagatgtgagacccctgtacagcagaagactgcccaagggagtgaagcacctgaaggacttccccatcctgcctggagagatcttcaagtacaagtggacagtgacagtggaggatggccccaccaagtctgaccccagatgcctgaccagatactacagcagctttgtgaacatggagagagacctggcctctggcctgattggccccctgctgatctgctacaaggagtctgtggaccagagaggcaaccagatcatgtctgacaagagaaatgtgatcctgttctctgtgtttgatgagaacagaagctggtacctgacagagaacatccagagattcctgcccaaccctgctggagtgcagctggaggaccctgagttccaggccagcaacatcatgcacagcatcaatggctatgtgtttgacagcctgcagctgtctgtgtgcctgcatgaggtggcctactggtacatcctgagcattggagcccagacagacttcctgtctgtgttcttctctggctacaccttcaagcacaagatggtgtatgaggacaccctgaccctgttccccttctctggagagacagtgttcatgagcatggagaaccctggcctgtggatcctgggctgccacaactctgacttcagaaacagaggcatgacagccctgctgaaggtgagcagctgtgacaagaacacaggagactactatgaggacagctatgaggacatctctgcctacctgctgagcaagaacaatgccattgagcccagaagcttcagccagaacccccctgtgctgaagagacaccagagagagatcaccagaaccaccctgcagtctgaccaggaggagattgactatgatgacaccatctctgtggagatgaagaaggaggactttgacatctatgatgaggatgagaaccagagccccagaagcttccagaagaagaccagacactacttcattgctgctgtggagagactgtgggactatggcatgagcagcagcccccatgtgctgagaaacagagcccagtctggctctgtgccccagttcaagaaggtggtgttccaggagttcacagatggcagcttcacccagcccctgtacagaggagagctgaatgagcacctgggcctgctgggcccctacatcagagctgaggtggaggacaacatcatggtgaccttcagaaaccaggccagcagaccctacagcttctacagcagcctgatcagctatgaggaggaccagagacagggagctgagcccagaaagaactttgtgaagcccaatgagaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcagacaggtgacagtgcaggagtttgccctgttcttcaccatctttgatgagaccaagagctggtacttcacagagaacatggagagaaactgcagagccccctgcaacatccagatggaggaccccaccttcaaggagaactacagattccatgccatcaatggctacatcatggacaccctgcctggcctggtgatggcccaggaccagagaatcagatggtacctgctgagcatgggcagcaatgagaacatccacagcatccacttctctggccatgtgttcacagtgagaaagaaggaggagtacaagatggccctgtacaacctgtaccctggagtgtttgagacagtggagatgctgcccagcaaggctggcatctggagagtggagtgcctgattggagagcacctgcatgctggcatgagcaccctgttcctggtgtacagcaacaagtgccagacccccctgggcatggcctctggccacatcagagacttccagatcacagcctctggccagtatggccagtgggcccccaagctggccagactgcactactctggcagcatcaatgcctggagcaccaaggagcccttcagctggatcaaggtggacctgctggcccccatgatcatccatggcatcaagacccagggagccagacagaagttcagcagcctgtacatcagccagttcatcatcatgtacagcctggatggcaagaagtggcagacctacagaggcaacagcacaggcaccctgatggtgttctttggcaatgtggacagctctggcatcaagcacaacatcttcaacccccccatcattgccagatacatcagactgcaccccacccactacagcatcagaagcaccctgagaatggagctgatgggctgtgacctgaacagctgcagcatgcccctgggcatggagagcaaggccatctctgatgcccagatcacagccagcagctacttcaccaacatgtttgccacctggagccccagcaaggccagactgcacctgcagggcagaagcaatgcctggagaccccaggtgaacaaccccaaggagtggctgcaggtggacttccagaagaccatgaaggtgacaggagtgaccacccagggagtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgagaatccacccccagagctgggtgcaccagattgccctgagaatggaggtgctgggctgtgaggcccaggacctgtactga

SEQ ID NO: 69 - DNA, 4398 bp, Homo sapiens
  misc_feature 1..4398: note = ATP7B sequence
  source 1..4398: mol_type = genomic DNA
atgcccgagcaggagcggcagatcacagccagagagggcgcctctaggaagatcctgtctaagctgagcctgccaaccagagcctgggagcctgccatgaagaagagcttcgcctttgataacgtgggatacgagggaggcctggacggactgggacctagctcccaggtggccacctccacagtgcgcatcctgggcatgacatgccagtcttgcgtgaagagcatcgaggatcggatctctaatctgaagggcatcatctccatgaaggtgtctctggagcagggcagcgccaccgtgaagtatgtgccatccgtggtgtgcctgcagcaggtgtgccaccagatcggcgacatgggcttcgaggcctctatcgcagagggcaaggcagccagctggccttctcgcagcctgccagcacaggaggcagtggtgaagctgagggtggagggcatgacatgccagagctgcgtgagcagcatcgagggcaaggtgagaaagctgcagggcgtggtgagggtgaaggtgtccctgtctaaccaggaggccgtgatcacctaccagccctatctgatccagcctgaggatctgagggaccacgtgaatgatatgggcttcgaggccgccatcaagtctaaggtggcaccactgagcctgggaccaatcgacatcgagcgcctgcagtccaccaacccaaagcggcccctgtcctctgccaaccagaacttcaacaattccgagacactgggccaccagggctctcacgtggtgaccctgcagctgcgcatcgatggcatgcactgcaagagctgcgtgctgaacatcgaggagaatatcggccagctgctgggcgtgcagagcatccaggtgtccctggagaacaagacagcccaggtgaagtacgacccttcctgcacctctccagtggccctgcagagggcaatcgaggccctgccccctggcaatttcaaggtgagcctgccagatggagcagagggctccggcaccgaccacagaagctcctctagccacagccctggctccccaccaaggaaccaggtgcagggcacatgtagcaccacactgatcgccatcgccggcatgacctgcgcctcttgcgtgcacagcatcgagggcatgatctcccagctggagggcgtgcagcagatcagcgtgtccctggccgagggcaccgcaacagtgctgtataatccctctgtgatcagccctgaggagctgagggcagcaatcgaggatatgggatttgaggcctccgtggtgtctgagagctgctccacaaaccccctgggcaatcactccgccggcaactctatggtgcagaccacagatggcacccccacaagcgtgcaggaggtggcacctcacaccggccgcctgccagcaaatcacgcaccagacatcctggccaagtccccacagtctacacgggccgtggcaccacagaagtgttttctgcagatcaagggcatgacctgcgccagctgcgtgagcaacatcgagagaaatctgcagaaggaggcaggcgtgctgtccgtgctggtggccctgatggcaggcaaggccgagatcaagtacgatcctgaagtgatccagccactggagatcgcccagttcatccaggacctgggctttgaggccgccgtgatggaggattatgccggcagcgacggcaacatcgagctgaccatcacaggcatgacctgcgcctcctgcgtgcacaacatcgagtctaagctgaccaggacaaatggcatcacatacgcctccgtggccctggccacctctaaggccctggtgaagttcgatcccgagatcatcggccctagagacatcatcaagatcatcgaggagatcggctttcacgcctccctggcccagaggaacccaaatgcccaccacctggatcacaagatggagatcaagcagtggaagaagagcttcctgtgctccctggtgtttggcatccccgtgatggccctgatgatctacatgctgatccctagcaacgagccacaccagagcatggtgctggaccacaacatcatccctggcctgagcatcctgaatctgatcttctttatcctgtgcacattcgtgcagctgctgggcggctggtacttttatgtgcaggcctataagagcctgcggcacagatccgccaatatggatgtgctgatcgtgctggccacctctatcgcctacgtgtatagcctggtcatcctggtggtggcagtggcagagaaggcagagagatcccccgtgaccttctttgacacacctccaatgctgttcgtgtttatcgccctgggcaggtggctggagcacctggccaagtctaagaccagcgaggccctggccaagctgatgagcctgcaggccacagaggccaccgtggtgacactgggcgaggataacctgatcatcagagaggagcaggtgcctatggagctggtgcagaggggcgacatcgtgaaggtggtgccaggcggcaagttccccgtggatggcaaggtgctggagggcaatacaatggccgacgagagcctgatcaccggagaggccatgcctgtgaccaagaagccaggctccacagtgatcgccggctctatcaacgcccacggcagcgtgctgatcaaggccacccacgtgggcaatgataccacactggcccagatcgtgaagctggtggaggaggcccagatgtccaaggcaccaatccagcagctggccgaccgcttcagcggctacttcgtgccttttatcatcatcatgtccaccctgacactggtggtgtggatcgtgatcggcttcatcgattttggcgtggtgcagagatattttcccaaccctaataagcacatcagccagaccgaagtgatcatcaggttcgcctttcagacctctatcacagtgctgtgcatcgcatgcccttgtagcctgggcctggccaccccaacagccgtgatggtgggcacaggagtggcagcacagaacggcatcctgatcaagggcggcaagcccctggagatggcccacaagatcaagaccgtgatgtttgataagaccggcacaatcacccacggcgtgcccagagtgatgagagtgctgctgctgggcgacgtggccacactgccactgaggaaggtgctggccgtggtgggcaccgcagaggcctcctctgagcacccactgggagtggccgtgacaaagtactgcaaggaggagctgggcacagagaccctgggctattgtaccgacttccaggccgtgcccggctgcggaatcggctgtaaggtgtctaacgtggagggcatcctggcacacagcgagagacccctgtctgcccctgccagccacctgaatgaggcaggctccctgccagcagagaaggatgccgtgccccagacattttctgtgctgatcggcaacagggagtggctgcggagaaatggcctgaccatcagctccgacgtgagcgacgccatgacagaccacgagatgaagggccagaccgccatcctggtggccatcgatggcgtgctgtgcggcatgatcgccatcgcagacgcagtgaagcaggaggccgccctggccgtgcacaccctgcagagcatgggcgtggatgtggtgctgatcaccggcgacaacaggaagacagccagagccatcgccacccaagtgggcatcaataaggtgttcgccgaggtgctgccttctcacaaggtggccaaggtgcaggagctgcagaacaagggcaagaaggtggctatggtgggcgatggcgtgaatgacagcccagccctggcccaggcagacatgggagtggcaatcggcacaggcaccgatgtggcaatcgaggcagcagacgtggtgctgatcagaaacgatctgctggacgtggtggcctccatccacctgtctaagcgcaccgtgaggcgcatccggatcaacctggtgctggccctgatctacaatctggtgggcatcccaatcgcagcaggcgtgtttatgcctatcggcatcgtgctgcagccctggatgggctccgccgctatggccgcctctagcgtgtctgtggtgctgtcctctctgcagctgaagtgctacaagaagcctgacctggagcggtacgaggcacaggcccacggccacatgaagccactgaccgcctcccaggtgtctgtgcacatcggcatggacgataggtggagggatagcccacgggccacaccctgggaccaggtgagctacgtgtcccaggtgagcctgagcagcctgaccagcgacaagcctagccgccacagcgccgccgccgacgatgacggcgataagtggtccctgctgctgaatggccgggacgaggagcagtatatctga

SEQ ID NO: 70 - DNA, 4398 bp, synthetic construct
  misc_feature 1..4398: note = Synthetic: Zero-CpG ATP7B sequence
  source 1..4398: mol_type = other DNA
atgcctgagcaggagagacagatcacagccagagagggagccagcagaaagatcctgagcaagctgagcctgcccaccagagcctgggagcctgccatgaagaagagctttgcctttgacaatgtgggctatgagggaggcctggatggcctgggccccagcagccaggtggccaccagcacagtgagaatcctgggcatgacctgccagagctgtgtgaagagcattgaggacagaatcagcaacctgaagggcatcatcagcatgaaggtgagcctggagcagggctctgccacagtgaagtatgtgccctctgtggtgtgcctgcagcaggtgtgccaccagattggagacatgggctttgaggccagcattgctgagggcaaggctgccagctggcccagcagaagcctgcctgcccaggaggctgtggtgaagctgagagtggagggcatgacctgccagagctgtgtgagcagcattgagggcaaggtgagaaagctgcagggagtggtgagagtgaaggtgagcctgagcaaccaggaggctgtgatcacctaccagccctacctgatccagcctgaggacctgagagaccatgtgaatgacatgggctttgaggctgccatcaagagcaaggtggcccccctgagcctgggccccattgacattgagagactgcagagcaccaaccccaagagacccctgagctctgccaaccagaacttcaacaactctgagaccctgggccaccagggcagccatgtggtgaccctgcagctgagaattgatggcatgcactgcaagagctgtgtgctgaacattgaggagaacattggccagctgctgggagtgcagagcatccaggtgagcctggagaacaagacagcccaggtgaagtatgaccccagctgcaccagccctgtggccctgcagagagccattgaggccctgccccctggcaacttcaaggtgagcctgcctgatggagctgagggctctggcacagaccacagaagcagcagcagccacagccctggcagcccccccagaaaccaggtgcagggcacctgcagcaccaccctgattgccattgctggcatgacctgtgccagctgtgtgcacagcattgagggcatgatcagccagctggagggagtgcagcagatctctgtgagcctggctgagggcacagccacagtgctgtacaacccctctgtgatcagccctgaggagctgagagctgccattgaggacatgggctttgaggcctctgtggtgtctgagagctgcagcaccaaccccctgggcaaccactctgctggcaacagcatggtgcagaccacagatggcacccccacctctgtgcaggaggtggccccccacacaggcagactgcctgccaaccatgcccctgacatcctggccaagagcccccagagcaccagagctgtggccccccagaagtgcttcctgcagatcaagggcatgacctgtgccagctgtgtgagcaacattgagagaaacctgcagaaggaggctggagtgctgtctgtgctggtggccctgatggctggcaaggctgagatcaagtatgaccctgaggtgatccagcccctggagattgcccagttcatccaggacctgggctttgaggctgctgtgatggaggactatgctggctctgatggcaacattgagctgaccatcacaggcatgacctgtgccagctgtgtgcacaacattgagagcaagctgaccagaaccaatggcatcacctatgcctctgtggccctggccaccagcaaggccctggtgaagtttgaccctgagatcattggccccagagacatcatcaagatcattgaggagattggcttccatgccagcctggcccagagaaaccccaatgcccaccacctggaccacaagatggagatcaagcagtggaagaagagcttcctgtgcagcctggtgtttggcatccctgtgatggccctgatgatctacatgctgatccccagcaatgagccccaccagagcatggtgctggaccacaacatcatccctggcctgagcatcctgaacctgatcttcttcatcctgtgcacctttgtgcagctgctgggaggctggtacttctatgtgcaggcctacaagagcctgagacacagatctgccaacatggatgtgctgattgtgctggccaccagcattgcctatgtgtacagcctggtgatcctggtggtggctgtggctgagaaggctgagagaagccctgtgaccttctttgacaccccccccatgctgtttgtgttcattgccctgggcagatggctggagcacctggccaagagcaagacctctgaggccctggccaagctgatgagcctgcaggccacagaggccacagtggtgaccctgggagaggacaacctgatcatcagagaggagcaggtgcccatggagctggtgcagagaggagacattgtgaaggtggtgcctggaggcaagttccctgtggatggcaaggtgctggagggcaacaccatggctgatgagagcctgatcacaggagaggccatgcctgtgaccaagaagcctggcagcacagtgattgctggcagcatcaatgcccatggctctgtgctgatcaaggccacccatgtgggcaatgacaccaccctggcccagattgtgaagctggtggaggaggcccagatgagcaaggcccccatccagcagctggctgacagattctctggctactttgtgcccttcatcatcatcatgagcaccctgaccctggtggtgtggattgtgattggcttcattgactttggagtggtgcagagatacttccccaaccccaacaagcacatcagccagacagaggtgatcatcagatttgccttccagaccagcatcacagtgctgtgcattgcctgcccctgcagcctgggcctggccacccccacagctgtgatggtgggcacaggagtggctgcccagaatggcatcctgatcaagggaggcaagcccctggagatggcccacaagatcaagacagtgatgtttgacaagacaggcaccatcacccatggagtgcccagagtgatgagagtgctgctgctgggagatgtggccaccctgcccctgagaaaggtgctggctgtggtgggcacagctgaggccagctctgagcaccccctgggagtggctgtgaccaagtactgcaaggaggagctgggcacagagaccctgggctactgcacagacttccaggctgtgcctggctgtggcattggctgcaaggtgagcaatgtggagggcatcctggcccactctgagagacccctgtctgcccctgccagccacctgaatgaggctggcagcctgcctgctgagaaggatgctgtgccccagaccttctctgtgctgattggcaacagagagtggctgagaagaaatggcctgaccatcagctctgatgtgtctgatgccatgacagaccatgagatgaagggccagacagccatcctggtggccattgatggagtgctgtgtggcatgattgccattgctgatgctgtgaagcaggaggctgccctggctgtgcacaccctgcagagcatgggagtggatgtggtgctgatcacaggagacaacagaaagacagccagagccattgccacccaggtgggcatcaacaaggtgtttgctgaggtgctgcccagccacaaggtggccaaggtgcaggagctgcagaacaagggcaagaaggtggccatggtgggagatggagtgaatgacagccctgccctggcccaggctgacatgggagtggccattggcacaggcacagatgtggccattgaggctgctgatgtggtgctgatcagaaatgacctgctggatgtggtggccagcatccacctgagcaagagaacagtgagaagaatcagaatcaacctggtgctggccctgatctacaacctggtgggcatccccattgctgctggagtgttcatgcccattggcattgtgctgcagccctggatgggctctgctgccatggctgccagctctgtgtctgtggtgctgagcagcctgcagctgaagtgctacaagaagcctgacctggagagatatgaggcccaggcccatggccacatgaagcccctgacagccagccaggtgtctgtgcacattggcatggatgacagatggagagacagccccagagccaccccctgggaccaggtgagctatgtgagccaggtgagcctgagcagcctgacctctgacaagcccagcagacactctgctgctgctgatgatgatggagacaagtggagcctgctgctgaatggcagagatgaggagcagtacatctga

SEQ ID NO: 71 - DNA, 652 bp, synthetic construct
  misc_feature 1..652: note = Synthetic: a minimized cruciform structure containing two inverted repeat sequences separated by a minimal R6K gamma origin
  source 1..652: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagcctgtcagccgttaagtgttcctgtgtcactcaaaattgctttgagaggctctaagggcttctcagtgcgttacatccctggcttgttgtccacaaccgttaaaccttaaaagctttaaaagccttatatattctttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcggctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 72 - DNA, 3775 bp, synthetic construct
  misc_feature 1..3775: note = Synthetic: Halo sTNALP vector comprising a minimized cruciform structure IR-R6K Ori-IR and EF1A-driven sTNALP-(DSS)6 transgene
  source 1..3775: mol_type = other DNA
ttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagctgccaccatggatatgagagtacctgctcaacttctgggtttgttgctgctttggctcaggggagccagatgtgctgccctggttcctgagaaggaaaaggaccccaagtactggagagatcaggcacaggagacactgaagtatgccctggagctgcagaagctgaataccaatgttgccaaaaatgtgatcatgttcctgggagatgggatgggagtcagcacagtgacagctgctagaatcctgaagggacagctgcatcataatcctggagaagagaccagactggaaatggataagttcccctttgtggccctgagcaagacatataataccaatgcccaagtgcctgattctgctggaacagctacagcttacctgtgtggagtgaaggccaatgagggaacagtgggagtgtctgctgccacagaaagaagcagatgcaacaccacccaaggcaatgaggtgacaagcatcctgagatgggccaaggatgctggcaaatctgtgggcattgtgacaacaaccagagtgaaccatgccaccccatctgctgcttatgcccattctgctgacagagactggtactctgacaatgagatgcctccagaggctctgtcccagggttgtaaagatattgcctatcagctgatgcacaacatcagagacattgatgtgatcatgggaggaggcaggaagtacatgtaccccaagaacaagacagatgtggagtatgagtctgatgagaaggccagaggcacaagactggatggcctggacctggtggacacctggaagtcttttaagcccagatacaagcacagccacttcatctggaacagaacagaactgctgaccctggacccccacaatgtggactacctgctgggcctgtttgagcctggagatatgcagtatgagctgaatagaaacaatgtgacagacccctccctgtctgaaatggtggtggtggccatccagatcctgagaaagaaccctaagggctttttcctgctggttgagggaggaagaattgaccatggccatcatgagggcaaggctaagcaggccctgcatgaggctgtggagatggacagagccattggccaagctggctctctgaccagctcagaggacaccctgacagtggtgacagctgatcactctcatgtgttcacttttggaggatatacccctagaggcaacagcatctttggcctggcccctatgctgtctgacacagacaaaaagcccttcacagccatcctgtatggcaatggcccaggatacaaggtggtgggaggagagagagagaatgtgtctatggtggactatgcacacaacaactaccaggcccagtctgctgttcctctgagacatgaaacacatggaggggaagatgtggcagtgttttctaaaggccctatggctcacctgctgcatggagtccatgagcagaactatgtgccccatgtgatggcctatgctgcctgcattggagcaaacctgggccactgtgcccctgccagctccgactcctctgattcttctgacagctcagactcctctgatagctctgacagctcttgagaattcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctccgcgcccggggagcccaaaggttaccccagttggggtacgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctgtcagccgttaagtgttcctgtgtcactcaaaattgctttgagaggctctaagggcttctcagtgcgttacatccctggcttgttgtccacaaccgttaaaccttaaaagctttaaaagccttatatattctttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcggctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcctggagacgttaattaaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcacc

SEQ ID NO: 73 - DNA, 3715 bp, synthetic construct
  misc_feature 1..3715: note = Synthetic: Halo sTNALP vector comprising a minimized cruciform structure IR-R6K Ori-IR and EF1A-driven cynomolgus monkey sTNALP transgene
  source 1..3715: mol_type = other DNA
ttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagctgccaccatgagagtgcctgcccagctgctgggcctgctgctgctgtggctgagaggagccagatgtgctgccctggtgcctgagaaggagaaggaccccaagtactggagagaccaggcccaggagaccctgaagtatgccctggagctgcagaagctgaacaccaatgtggccaagaatgtgatcatgttcctgggagatggcatgggagtgagcacagtgacagccaccagaatcctgaagggccagctgcaccacaaccctggagaggagaccagactggagatggacaagttcccctttgtggccctgagcaagacctacaacaccaatgcccaggtgcctgactctgctggcacagccacagcctacctgtgtggagtgaaggccaatgagggcacagtgggagtgtctgctgccacagagagaagcagatgcaacaccacccagggcaatgaggtgaccagcatcctgagatgggccaaggatgctggcaagtctgtgggcattgtgaccaccaccagagtgaaccatgccaccccctctgctgcctatgcccactctgctgacagagactggtactctgacaatgagatgccccctgaggccctgagccagggctgcaaggacattgcctaccagctggtgcacaacatcagagacattgatgtgatcatgggaggaggcagaaagtacatgtaccccaagaacaagacagatgtggagtatgagattgatgagaaggccagaggcaccagactggatggcctggacctggtgaacatctggaagagcttcaagcccagacacaagcacagccacttcatctggaacagaacagagctgctgaccctggacccccacaatgtggactacctgctgggcctgtttgagcctggagacatggagtatgagctgaacagaaacaatgtgacagaccccagcctgtctgagatggtggtggtggccatccagatcctgagaaagaaccccaagggcttcttcctgctggtggagggaggcagaattgaccatggccaccatgagggcaaggccaagcaggccctgcatgaggctgtggagatggacagagccattggccaggctggcagcatgaccagcctggaggacaccctgacagtggtgacagctgaccacagccatgtgttcacctttggaggctacacccccagaggcaacagcatctttggcctggcccccatgctgtctgacacagacaagaagcccttcacagccatcctgtatggcaatggccctggctacaaggtggtgggaggagagagagagaatgtgagcatggtggactatgcccacaacaactaccaggcccagtctgctgtgcccctgagacatgagacccatggaggagaggatgtggctgtgttcagcaagggccccatggcccacctgctgcatggagtgcatgagcagaactacatcccccatgtgatggcctatgctgcctgcattggagccaacctggaccactgtgcccctgccagcagctgagaattcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctccgcgcccggggagcccaaaggttaccccagttggggtacgtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctgtcagccgttaagtgttcctgtgtcactcaaaattgctttgagaggctctaagggcttctcagtgcgttacatccctggcttgttgtccacaaccgttaaaccttaaaagctttaaaagccttatatattctttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatcggctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcctggagacgttaattaaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcacc

SEQ ID NO: 74 - DNA, 41 bp, synthetic construct
  misc_feature 1..41: note = Synthetic: synthetic dna
  source 1..41: mol_type = other DNA
ttggccactccctctctgcgcgctdgctcgctcactgaggc

SEQ ID NO: 75 - DNA, 41 bp, synthetic construct
  misc_feature 1..41: note = Synthetic: synthetic dna
  source 1..41: mol_type = other DNA
gcctcagtgagcgagcgagcgcgcagagagggagtggccaa

SEQ ID NO: 76-79 - skipped (4 entries)

SEQ ID NO: 80 - DNA, 20 bp, synthetic construct
  misc_feature 1..20: note = Synthetic: synthetic dna
  source 1..20: mol_type = other DNA
aggaacccctagtgatggag

SEQ ID NO: 81 - DNA, 20 bp, synthetic construct
  misc_feature 1..20: note = Synthetic: synthetic dna
  source 1..20: mol_type = other DNA
ctccatcactaggggttcct

SEQ ID NO: 82 - DNA, 1257 bp, synthetic construct
  misc_feature 1..1257: note = Synthetic: Zero-CpG Alpha-1 anti-trypsin sequence
  source 1..1257: mol_type = other DNA
atgcccagctctgtgagctggggcatcctgctgctggctggcctgtgctgcctggtgcctgtgagcctggctgaggacccccagggagatgctgcccagaagacagacaccagccaccatgaccaggaccaccccaccttcaacaagatcacccccaacctggctgagtttgccttcagcctgtacagacagctggcccaccagagcaacagcaccaacatcttcttcagccctgtgagcattgccacagcctttgccatgctgagcctgggcaccaaggctgacacccatgatgagatcctggagggcctgaacttcaacctgacagagatccctgaggcccagatccatgagggcttccaggagctgctgagaaccctgaaccagcctgacagccagctgcagctgaccacaggcaatggcctgttcctgtctgagggcctgaagctggtggacaagttcctggaggatgtgaagaagctgtaccactctgaggccttcacagtgaactttggagacacagaggaggccaagaagcagatcaatgactatgtggagaagggcacccagggcaagattgtggacctggtgaaggagctggacagagacacagtgtttgccctggtgaactacatcttcttcaagggcaagtgggagagaccctttgaggtgaaggacacagaggaggaggacttccatgtggaccaggtgaccacagtgaaggtgcccatgatgaagagactgggcatgttcaacatccagcactgcaagaagctgagcagctgggtgctgctgatgaagtacctgggcaatgccacagccatcttcttcctgcctgatgagggcaagctgcagcacctggagaatgagctgacccatgacatcatcaccaagttcctggagaatgaggacagaagatctgccagcctgcacctgcccaagctgagcatcacaggcacctatgacctgaagtctgtgctgggccagctgggcatcaccaaggtgttcagcaatggagctgacctgtctggagtgacagaggaggcccccctgaagctgagcaaggctgtgcacaaggctgtgctgaccattgatgagaagggcacagaggctgctggagccatgttcctggaggccatccccatgagcatcccccctgaggtgaagttcaacaagccctttgtgttcctgatgattgagcagaacaccaagagccccctgttcatgggcaaggtggtgaaccccacccagaagtga

SEQ ID NO: 83 - protein, 1465 aa, Homo sapiens
  REGION 1..1465: note = misc_feature - Full length ATP7B amino acid sequence
MPEQERQITAREGASRKILSKLSLPTRAWEPAMKKSFAFDNVGYEGGLDGLGPSSQVATSTVRILGMTCQSCVKSIEDRISNLKGIISMKVSLEQGSATVKYVPSVVCLQQVCHQIGDMGFEASIAEGKAASWPSRSLPAQEAVVKLRVEGMTCQSCVSSIEGKVRKLQGVVRVKVSLSNQEAVITYQPYLIQPEDLRDHVNDMGFEAAIKSKVAPLSLGPIDIERLQSTNPKRPLSSANQNFNNSETLGHQGSHVVTLQLRIDGMHCKSCVLNIEENIGQLLGVQSIQVSLENKTAQVKYDPSCTSPVALQRAIEALPPGNFKVSLPDGAEGSGTDHRSSSSHSPGSPPRNQVQGTCSTTLIAIAGMTCASCVHSIEGMISQLEGVQQISVSLAEGTATVLYNPSVISPEELRAAIEDMGFEASVVSESCSTNPLGNHSAGNSMVQTTDGTPTSVQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI